(19) United States
(12) Patent Application Publication
(10) Pub. No.: US 2025/0064369 A1
(43) Pub. Date: Feb. 27, 2025

(54) MICROELECTRODE FOR INSERTION INTO SOFT TISSUE

(71) Applicant: NEURONANO AB, Karlshamn (SE)

(72) Inventor: Jens SCHOUENBORG, Lund (SE)

(21) Appl. No.: 18/726,323

(22) PCT Filed: Jan. 5, 2023
(86) PCT No.: PCT/SE2023/050008

§ 371 Date: Jul. 2, 2024

(30) Foreign Application Priority Data
Jan. 5, 2022 (SE) 2230001-6
Mar. 14, 2022 (SE) 2230072-7

(51) Int. Cl. A61B 5/27; A61B 5/268

(52) U.S. Cl. CPC A61B 5/27; A61B 5/268; A61B 2562/125

(57) ABSTRACT

A microelectrode that includes an electrically conductive element, the electrically conductive element having a proximal electrically insulated portion and distal non-insulated portion, at least part of the conductive element being disposed in a casing of electrically insulating non-degradable material having a first structural component and optionally a second structural component, the distal non-insulated portion of the conductive element being encapsulated (surrounded) by the casing forming a distal chamber, a void/lumen being present between the insulated portion of the conductive element and the first structural component the void/lumen enabling the conductive element to slide with respect to the casing, the casing of the distal chamber having at least one electrically conductive bridge electrically coupling the distal chamber with the adjacent soft tissue.

### FIELD OF THE INVENTION

The present invention relates to a microelectrode configured to be at least partly embedded in soft tissue or at least partly placed adjacent to soft tissue, specifically nervous, endocrine, muscle and connective tissue, comprising an elongated (oblong) electrically conductive element (electrode) comprising insulated and non-insulated portions. At least a non-insulated portion of the conductive element is disposed within a casing (envelope, sleeve, sheath) of an electrically insulating non-degradable material forming a chamber. The casing of the chamber is configured to electrically couple (connect) the non-insulated portion of the conductive element with the soft tissue. The electrical coupling may be achieved by electric conductive bridges such as fluidic and non-fluidic conductive bridges. A characterizing feature of the microelectrode is that the casing is disengaged with respect to the conductive element. Thus, the casing and conductive element can move with respect to each other. The casing is movably disposed around the conductive element by at least a first structural component which may form part of the casing or is a separate part. The invention further encompasses a microelectrode probe, arrays of microelectrodes and/or microelectrode probes and a method for the manufacturing of the microelectrode/microelectrode probe. The various aspects of the present invention are preferably applied for neuromodulation and sensing of neural, endocrine and muscular functions but can also be used for localized drug delivery.

### BACKGROUND AND OBJECTIVES

Implantable microelectrodes and sets of microelectrodes have a wide scope of applications in human and veterinary medicine.

A microelectrode implanted into nervous, endocrine or muscle tissue, independent on whether constituting a single implant or pertaining to an implant comprising multiple microelectrodes such as a bundle or array of microelectrodes, requires connection to control device(s) disposed exteriorly of the tissue to be monitored and/or stimulated.

This connection is generally provided by thin insulated flexible electrical leads. The leads bridge tissues of various kind and stiffness and thereby become affected by their recurrent displacement relative to each other caused by for example breathing, heart beets, head and spine movements, position of the brain relative to the skull, and age-related changes. This kind of tissue movement may similarly affect other thin and flexible implants such as microfibers or microfilaments, in particular optical, microelectrodes and catheters.

An example of a situation in which movements of tissues relative to each other can be observed is when an electrical lead bridges the skull and the brain via a space comprising dura mater, arachnoid membrane, cerebrospinal fluid, and pia mater. Other examples are leads bridging vertebrae and spinal cord; muscle and adjacent fibrous sheets and connective tissue; peripheral nerves (such as the vagus nerve) and surrounding tissue. These movements of tissues relative to each other result in different forces (for example shear forces and tear forces) acting between implanted leads and tissue at their bordering area, which risk causing persistent local inflammation and tissue injury. In addition, forces and for example shear forces of this kind may affect the position of the electric contact(s) (i.e. the non-insulated portion(s)), of an implanted microelectrode with respect to surrounding cells/tissue. Instable position of the electrode contact(s) with respect to targeted tissue also results in variability of the specific neuronal, endocrine or muscle elements being recorded or stimulated over time, which is especially problematic when monitoring and analyzing long term changes in such signals or when stable long-term stimulation of such elements are necessary.

WO 2022005386A1 discloses a microelectrode comprising a casing disposed around the conductive electrode where a distal non-insulated section of the conductive electrode is encapsulated by the casing forming a distal chamber. The casing is slidably mounted around the conductive electrode. Moreover, the casing has an opening providing a fluidic electrical coupling. WO 2022005386A1 does not disclose to electrically couple the encapsulated electrode with the soft tissue without the exchange of charged particles (ions) across the casing. Furthermore, WO 2022005386A1 fails to disclose a microelectrode comprising a casing, first and second structural components where the casing, first and second structural components form a distal chamber surrounding a non-insulated portion of an electrode where the casing has an opening distally to the second structural component. Additionally, WO 2022005386A1 fails to disclose a microelectrode comprising a non-insulated electrode portion confined within a chamber restricted by a casing, first and second structural components said non-insulated electrode portion being disposed in an inner casing of an electrically conductive non-degradable material.

The microelectrodes of WO 2022005386A1 provide a configuration encapsulating a conductive element from the adjacent soft tissue by a casing of a flexible, non-degradable material, the casing comprising one or more openings through which an electric contact with the tissue is established by migration of electrically charged particles (ions). Furthermore, the casing is detached from the conductive element enabling the casing to move with respect to the conductive element. The detachment of the casing from the conductive element significantly improves the positional stability of the opening(s) in the casing with respect to surrounding tissue and thereby also specificity and resolution of recordings and stimulations. Over time, however, adjacent tissue has a tendency not only to penetrate the casing through the openings but also to grow inside the chamber to the point that soft tissue attaches to the non-insulated conductive element. Once soft tissue attaches to the non-insulated conductive element (which exhibits sliding movements inside the casing) positional stability of the recorded/stimulated tissue is compromised. Movement of the non-insulated portion of the conductive element connected to tissue also cases tissue adjacent an opening and outside the casing to move causing variations in signal-to-noise ratio and wave-shape of recorded neuronal signals and variable stimulation efficacy. The casing of the microelectrodes of WO 2022005386A1 is also associated with the conductive element such that the distal tip of the non-insulated portion of the conductive element never touch or penetrate the casing. An embodiment of the microelectrode of WO 2022005386A1 comprises a first and second structural component separate from the casing, The casing present distally to the second structural component is closed. A movement of the conductive element in distal direction will increase the pressure in the casing and where applicable also in the casing distally to an optional second structural component. Correspondingly, a movement of the conductive element in proximal direction will decrease the pressure of said volume and where applicable also in the casing distally to an optional second structural component. These changes in pressure can affect the positional stability of the tissue immediately outside an opening in the casing.

Some embodiments of the herein disclosed microelectrode comprise an elongated electrically conductive element comprising a proximal and distal end, the electrically conductive element comprising insulated proximal and distal portions, the proximal insulated portion extending in distal direction from the proximal end, the distal insulated portion extending in proximal direction from the distal end, the proximal and distal portions separated by a non-insulated portion of the conductive element, at least the non-insulated portion of the conductive element being essentially centrally disposed in a casing of an electrically insulating non-degradable material, the microelectrode further comprising first and second structural components, said first and second structural components extending in radial direction between the casing and the conductive element, the first structural component movably disposed around the proximal insulated portion, the second structural component movably disposed around the distal insulated portion; the casing, first and second structural components forming a distal chamber, wherein the casing has an opening distally to the second structural component, wherein the casing is configured to electrically couple the non-insulated portion of the conductive element with the soft tissue. Such embodiments improve the axial adjustability of the conductive element with respect to the casing in view of the microelectrodes of WO 2022005386A1 as the opening in the casing distally to the second structural component prevents pressure changes when the conductive element is moving in respect of the casing. Hence, these embodiments improve the freedom of movement of the conductive element with respect to the casing thereby further improving positional stabilization of the casing at the site(s) where the non-insulated portion of the conductive element is (are) on contact with adjacent tissue thereby improving the positional stability of the electric coupling of the casing with respect to the surrounding tissue and specifically specificity and resolution of recordings and stimulations.

An objective of the present invention in respect of prior art and specifically WO 2022005386A1 is to improve or even maintain the positional stabilization in the targeted tissue of the casing and by inference the electric contact(s) of an implanted microelectrode over time, and preferably maintaining positional stabilization of the electric contact(s) over the lifespan of an implantable microelectrode. A further object of the invention is to provide a microelectrode that can be at least partially embedded (implanted) in soft tissue or at least partly placed adjacent to soft tissue, in particular nervous, muscle or endocrine tissue, and electrically connected with a control apparatus for recording and stimulation purposes disposed exteriorly of the target tissue, which avoids or at least reduces tissue irritation by movements of tissue abutting the microelectrode or abutting a lead electrically connecting the microelectrode with an electrode control apparatus disposed outside the tissue of implantation.

Another object of the invention is to prevent or reduce unintended dislocations in the targeted tissue of an implanted microelectrode's electric contact by forces affecting the lead by which the microelectrode is electrically connected to an electronic control apparatus for recordings and/or stimulations.

Still another object of the invention is to provide for increased freedom of lateral movement of an implanted microelectrode.

A further object of the invention is to provide a microelectrode probe or an array of such probes for implantation into soft tissue or at least partly placed adjacent to soft tissue, in particular nervous, muscular or endocrine tissue, capable of being transformed to a microelectrode or an array of microelectrodes by contact with aqueous body fluid.

Still a further object is the provision of a microelectrode or array of microelectrodes for placement on the surface (or adjacent to the surface) of the brain, spinal cord, dorsal root ganglia, peripheral nerves, endocrine organs or muscles for monitoring and/or stimulation of said structures.

An additional object of the invention is to provide methods of manufacture for a microelectrode probe and an array of microelectrode probes of the invention.

A further objective is the provision of drug delivery to same or essentially the same tissue from a flexible compartment implanted into soft tissue.

Another objective of the present invention is to avoid or minimize the physical contact of the conductive element, specifically the non-insulated portion of the element, with adjacent soft tissue, while allowing the conductive element (including the non-insulated and insulated portions) to move inside the casing to positionally stabilize the casing with respect to the surrounding tissue.

Other objects will be apparent from the description below.

### Explanation of Some General Terms

The following terms are repeatedly used to define embodiments of the invention

### Spatial Terms ‘Distal’ and ‘Proximal’

The terms ‘proximal’ and ‘distal’ are used to specify entities of the different aspects of the invention in relation to other optional devices electrically connected to a microelectrode and positioned outside the target tissue (the target tissue being where the stimulation/recordings are to be made). A proximal entity or a proximal part/portion/section/region of an entity, e.g. relating to the microelectrode, is closer (with respect to the length of the connecting microelectrode/lead) to an optional electrical device than a distal entity or a distal part/portion/section/region of an entity. The transition from a proximal part/portion/section/region of an entity to a distal part/portion/section/region of an entity should not be understood as a very specific region, rather, the division of an entity or designation of entities into/as proximal and distal is a means to position such entities in relation to each other. Within the context of the present invention another optional device usually is an electrode control apparatus disposed outside the tissue of implantation electrically connected by a lead to a microelectrode. The point of connection of the lead to a microelectrode defines the proximal zone of said microelectrode. The opposite end of a microelectrode with respect to the point of lead connection is consequently the distal end of the microelectrode.

### Microelectrode

Within the context of the present disclosure a microelectrode is a device for the purpose of registering/recording/monitoring or stimulating various soft tissues including nervous tissue, endocrine tissue, exocrine tissue, muscular tissue, heart tissue, connective tissue and retina. The microelectrode comprises an elongated (oblong) electrically conductive element (conductive electrode) which is partly electrically insulated. At least one non-insulated portion of the electrically conductive element is disposed in a casing of an electrically insulating, non-degradable material, the casing comprising at least one first structural component which structural component(s) may form part om the casing. The casing, first and optionally second structural component(s) enclose/encapsulates a non-insulated portion of the conductive element forming a chamber. The first and optionally second structural component(s) movably engages the casing with respect to the conductive element. Typically, the first and optionally second structural component(s) movably engages with an insulated portion of the conductive element. In some embodiments, at least part of the first structural component is movably disposed around a proximal insulated portion of the conductive element. In some embodiments, at least part of the first structural component is movably disposed around a proximal insulated portion of the conductive element and at least a second structural component is movably disposed around a distal insulated portion of the conductive element. The inner diameter of the first and optional second structural component(s) is/are equal to or preferably greater than the diameter of the insulated portion(s) of the conductive element. The casing, first and optionally second structural component(s) substantially enclose (encapsulates) a non-insulated portion of the conductive element forming a chamber, in some embodiment the chamber is referred to as a distal chamber. By enclosure or encapsulation should be understood that the casing, first structural component and optionally second structural essentially partition the chamber from soft tissue when the microelectrode is positioned adjacent or into soft tissue. Essentially partitioning the chamber from soft tissue signifies that the casing reduces or eliminates the ability of tissue to penetrate the casing and growing inside the distal chamber.

In some embodiment, the first structural component partitions the casing in a distal chamber and a proximal compartment.

An important feature of the microelectrode is that the casing can move with respect to the elongated electrically conductive element. Thus, the casing is not permanently attached to the conductive element. Instead, the casing is detached, separated from the conductive element enabling the casing to move predominately in axial direction with respect to the conductive element.

The microelectrode is preferably flexible. A flexible microelectrode is capable to accommodate for movements of surrounding tissue. Flexibility of the microelectrode is in part provided by the flexible casing, in part by the detachment of the casing from the conductive element and in part of the flexibility of the conductive element. Radial flexibility of the microelectrode is in part provided by the flexibility of the casing and also the detachment of the casing from the conductive element, axial flexibility of the microelectrode is provided by the ability of the casing to move with respect to the conductive element. Usually, a flexible microelectrode is difficult or even impossible to implant into soft tissue without using stiffening providing components. Hence, the term flexibility may also include the inability to successfully implant a microelectrode into soft tissue.

The main axis of a microelectrode is collinear with the elongated conductive element, suitably coinciding with the elongated conductive element, the conductive element preferably being essentially centrally disposed within the casing.

### Microelectrode Probe

A microelectrode probe is a microelectrode which is more easily implantable into tissue typically, the microelectrode probe comprising materials providing enhanced structural rigidity during insertion into soft tissue but disintegrates and/or dissolves upon insertion into soft tissue.

Consequently, a microelectrode or array as disclosed herein generally does not comprise structural features which do not disintegrate and/or dissolves upon insertion into soft tissue.

### First and Optionally Second Structural Components

The casing comprises at least a first structural component and optionally a second structural component. In some embodiments, the first and optional second structural component may form part of the casing, being an integral component of the casing.

Alternatively, and in some embodiments, the first and optional second structural component are separate, distinct from the casing. If first and optionally second structural components are separate from the casing, they are preferably made of a different material than the material of the casing.

First and optional second structural components preferably extend in radial direction between the casing and the conductive element, typically between the casing and insulated portions of the conductive element.

If the first and optional second structural components are an integral part of the casing there must be a lumen/space, suitably an annular gap, between the first, optionally second structural component(s) and the conductive element, preferably between the first, optional second structural component(s) and an insulated portion of the conductive element.

In case the first and optional second structural component(s) is/are separate from the casing, the first, and optional second structural components may be permanently engaged with the insulated portion(s) of the conductive element while the casing is detached (separated) from the first and optional second structural components allowing the casing to move predominantly in axial direction with respect to the first and optional second structural component(s) and by inference also with respect to the conductive element. Alternatively, the first and optional second structural component(s) is/are permanently attached to the casing while detached from the insulated portion(s) of the conductive element.

Substantial enclosure or encapsulation of a non-insulated portion of the conductive element herein means that the casing is configured to minimize or even eliminate the growth of tissue inside the distal chamber while at the same time enabling the non-insulated portion of the conductive element to electrically engage/couple with the tissue surrounding the casing.

The center of the first and second structural components are preferably linearly aligned along the main axis.

### Distal Chamber, Proximal Compartment

A distal chamber is a volume formed by the casing of electrically insulating non-degradable material (which also may be referred to as envelope, sleeve, sheath) and at least a first structural component enclosing or encapsulating a non-insulated (axial) portion of the conductive element. The distal chamber is extending distally from the first structural component. For embodiments comprising a first and second structural components the distal chamber is a volume formed (restricted) by the casing and first and second structural components. For embodiments comprising only a first structural components the distal casing is formed (restricted) by the casing and the first structural components.

The distal chamber is preferably essentially electrically insulated from adjacent soft tissue except for the electrical coupling comprised in the casing, such as at least one conductive bridge. As the casing is detached from the conductive element as further elaborated herein, some current will inevitably leak trough the gaps between first and optionally second structural components and the conductive element or between first and optionally second structural components and casing depending on embodiment. In some embodiments the first structural component partitions the casing in a distal chamber and proximal compartment. The volume of a proximal compartment extends proximally with respect to the first structural component. Suitably, a proximal compartment accommodates an insulated portion of the conductive element.

### Non-Degradable Material

Some parts of the microelectrode, microelectrode probe, arrays, such as casing and insulation of the conductive element are of non-degradable material. The microelectrodes are positioned within tissue of animals and humans. Tissues contain various compounds with can have an impact of materials of the microelectrode. A non-degradable material is a material which throughout the lifetime of an implanted microelectrode essentially retains its indented function. The lifespan of an implanted microelectrode is several years up to several decades. Hence, a non-degradable material retains its function for the lifespan of the microelectrode, typically at least 1, 2, 3, 4, 5, 10, 15, 20 years.

### Inner Casing

Some embodiments of the microelectrode comprise an inner casing. The inner casing is disposed around the non-insulated conductive element and inside the chamber formed by the casing and at least one structural component. The material of the inner casing is electrically conductive and preferably non-degradable. The outer diameter of the inner casing is equal to or smaller than the inner diameter of the casing. The inner diameter of the inner casing is larger than the diameters of the distal and proximal insulated portions of the conductive elements. The inner casing has preferably an annular cross-section. The purpose of the inner casing is to prohibit or at least significantly reducing the likelihood of tissue attaching to the non-insulated conductive element.

### Electrical Coupling

Electrical coupling is the ability to electrically couple an electrode enclosed in a casing with soft tissue adjacent the casing. An enclosed non-insulated portion of the conductive element can electrically interact with adjacent soft tissue by a conductive bridge selected from any one or both of: a) the exchange of charged particle across the casing, herein referred to as a fluidic electrically conductive bridge, or, b) by electrically couple the enclosed electrode with the soft tissue without the exchange of charged particles (ions and electrons) across the casing, herein referred to as an electric conductive bridge (also non-fluidic electric conductive bridge).

### Electric Conductive Bridge

An electric conductive bridge as used herein is an entity which enables an enclosed non-insulated portion of the conductive element to electrically interact with adjacent soft tissue without the exchange of charges particles. An electric conductive bridge may also be configured to exchange charged particles across the casing. An example of an electric conductive bridge also exchanging charged particles across the casing are hollow conductive filaments or a conductive mesh or net. Electric conductive bridges lacking the ability to also exchange charged particle across the casing are denoted non-fluidic electrically conductive bridges.

### Soft Tissue

In its widest definition soft tissue relates to soft tissue of any sentient beings excluding hard tissue such as osseous (bone) tissue. More particularly, soft tissue encompasses any soft tissue which provides electric fingerprints which can be monitored and/or any tissue susceptible to electric stimulation. Specifically interesting soft tissue sub-groups constitutes nervous tissue, endocrine tissue, muscle tissue, connective tissue, and retina (tissue). Soft tissue also encompasses hollow fluidic spaces such as ventricles.

The terms ‘embedded’ and ‘implanted’ can be used interchangeably.

The present invention relates to a microelectrode, a microelectrode probe, different arrays of microelectrodes and/or microelectrode probes, and a method for the manufacturing of a microelectrode, a microelectrode probe and arrays.

### Overview of Some Embodiments of the Microelectrode

All embodiments of the microelectrode disclosed herein share the following general features:
A non-insulated portion of an elongated conductive element also comprising an insulated portion disposed within a casing of a flexible, non-degradable material.
At least a first structural component which may form part of the casing, alternatively, a first structural component as a distinct entity, separate from the casing.
The casing movably associated with the conductive element.

Any one and any number of the preferred features of any one of the embodiments in this section may be combined with any one of the embodiments as long as a preferred feature does not introduce inconsistencies.

Some embodiments (denoted A) comprise the following features in addition to the general features:
An elongated conductive element comprising a distal non-insulated portion and a proximal insulated portion.
A first structural component but not a second structural component.
At least the non-insulated portion of the conductive element being disposed within a casing of a flexible, non-degradable material.
The casing and the first structural component forming a distal chamber.
The casing comprising electrically conductive bridges electrically coupling the non-insulated portion of the conductive element with soft tissue adjacent the distal chamber and outside the casing.

Any and any number of the below features may be combined with the above embodiment A features:

The casing and the first structural component essentially electrically isolating/insulating the distal chamber from the adjacent soft tissue except for the electrically conductive bridges.

The distal non-insulated portion extending in proximal direction from the distal end until the proximal insulated portion.

The distal non-insulated portion extending in proximal direction from the distal end until the proximal insulated portion being essentially electrically insulated from the soft tissue by the casing and the first structural component except for the electrically conductive bridges.

The conductive element being essentially centrally disposed within the casing by the first structural component.

The first structural component extending in radial direction between the casing and the proximal insulated portion of the conductive element.

The first structural component not forming part of the casing.

The first structural component being movably disposed around the insulated portion of the conductive element.

The first structural component being movably disposed around the insulated portion of the conductive element and permanently attached to the casing.

The first structural component being permanently attached to the insulated portion of the conductive element and the casing movably disposed around the first structural component.

The first structural component being permanently engaged with the casing to provide electrical insulation.

The first structural component having an annular cross-section.

The first structural component having an annular cross-section with an inner diameter larger than the diameter of the proximal insulated portion of the conductive element and preferably an outer diameter essentially same as inner diameter of the casing.

At least part of the non-insulated portion of the conductive element being disposed in an inner casing of an electrically conductive, non-degradable material, the inner casing having an outer diameter which is equal to or smaller than the inner diameter of the casing.

Some embodiments (denoted B) comprise the following features in addition to the general features:
An elongated conductive element comprising a distal non-insulated portion and a proximal insulated portion.
A first and second structural component.
At least the non-insulated portion of the conductive element being disposed within a casing of a flexible, non-degradable material.
The casing, first and second structural component forming a distal chamber.
The casing comprising electrically conductive bridges electrically coupling the non-insulated portion of the conductive element with soft tissue adjacent the distal chamber and outside the casing.

Any and any number of the below features may be combined with the above embodiment B features:

The casing, the first and second structural component essentially electrically isolating/insulating the distal chamber from the adjacent soft tissue except for the electrically conductive bridges.

The distal non-insulated portion extending in proximal direction from the distal end until the proximal insulated portion.

The distal non-insulated portion extending in proximal direction from the distal end until the proximal insulated portion being essentially electrically insulated from the soft tissue by the casing and the first structural component except for the electrically conductive bridges.

The conductive element being essentially centrally disposed within the casing by the first and second structural components.

The first and second structural components not forming part of the casing.

The first structural component extending in radial direction between the casing and the insulated portion of the conductive element.

The second structural component extending in radial direction between the casing and the distal non-insulated portion of the conductive element

The first structural component being movably disposed around the proximal insulated portion of the conductive element.

The second structural component being movably disposed around the distal non-insulated portion of the conductive element.

The first and second structural components being permanently engaged with the casing to provide electrical insulation.

The first and second structural components having annular cross-sections.

The center of the first and second structural components being linearly aligned along the main axis.

The first structural component having an annular cross-section with an inner diameter larger than the diameter of the proximal insulated portion of the conductive element and preferably an outer diameter essentially same as inner diameter of casing.

The second structural component having an annular cross-section with an inner diameter larger than the diameter of the distal non-insulated portion of the conductive element and preferably an outer diameter essentially same as inner diameter of the casing.

At least part of the non-insulated portion of the conductive element being disposed in an inner casing of an electrically conductive, non-degradable material, the inner casing having an outer diameter which is equal to or smaller than the inner diameter of the casing.

Some embodiments (denoted C) comprise the following features in addition to the general features:
An elongated conductive element having a proximal and distal end, comprising insulated proximal and distal portions, said proximal insulated portion extending in distal direction from the proximal end, said distal insulated portion extending in proximal direction from the distal end, the proximal and distal portions separated by a non-insulated portion of the conductive element.
A first and second structural component.
At least the non-insulated portion of the conductive element being disposed within a casing of a flexible, non-degradable material.
The casing and the first structural component forming a distal chamber.
The casing comprising electrically conductive bridges electrically coupling the non-insulated portion of the conductive element with soft tissue adjacent the distal chamber and outside the casing.
The insulated distal portion of the conductive element protruding through an opening in the distal end of the casing, the opening in the distal end of the casing acting as radially stabilizing the distal insulated portion of the conductive element.
The distal end of the casing and the opening acting as second structural component.

Any and any number of the below features may be combined with the above embodiment C features:

The casing and the first and second structural components essentially electrically isolating/insulating the distal chamber from the adjacent soft tissue except for the electrically conductive bridges.

The conductive element being essentially centrally disposed within the casing by the first and second structural component.

The first and second structural components extending in radial direction between the casing and the proximal and distal insulated portion of the conductive element.

The first and second structural components being movably disposed around the insulated portions of the conductive element.

The first structural component not forming part of the casing.

The first structural component being movably disposed around the proximal insulated portion of the conductive element and permanently attached to the casing.

The first structural component being permanently attached to the proximal insulated portion of the conductive element and the casing movably disposed around the first structural component.

The first structural component being permanently engaged with the casing to provide electrical insulation.

The first structural component having an annular cross-section.

The first structural component having an annular cross-section with an inner diameter larger than the diameter of the proximal insulated portion of the conductive element and preferably an outer diameter essentially same as inner diameter of the casing.

The diameter of the opening in the distal end of the casing being larger than the diameter of the distal insulated portion of the conductive element.

At least part of the non-insulated portion of the conductive element being disposed in an inner casing of an electrically conductive, non-degradable material, the inner casing having an outer diameter which is equal to or smaller than the inner diameter of the casing.

Some embodiments (denoted D) comprise the following features in addition to the general features:
An elongated conductive element having a proximal and distal end, comprising insulated proximal and distal portions, said proximal insulated portion extending in distal direction from the proximal end, said distal insulated portion extending in proximal direction from the distal end, the proximal and distal portions separated by a non-insulated portion of the conductive element.
A first and second structural component.
At least the non-insulated portion of the conductive element being disposed within a casing of a flexible, non-degradable material.
The casing, the first and second structural component forming a distal chamber.
The casing comprising an opening distally to the second structural component.
The casing comprising electrically conductive bridges electrically coupling the non-insulated portion of the conductive element with soft tissue adjacent the distal chamber and outside the casing.

Any and any number of the below features may be combined with the above embodiment D features:

The conductive element being essentially centrally disposed within the casing by the first and second structural components.

The first and second structural components extending in radial direction between the casing and the insulated proximal portion and distal insulated portion respectively of the conductive element.

The first and second structural components not forming part of the casing.

The first and second structural components being movably disposed around the proximal insulated and distal insulated portions respectively of the conductive element.

The first and second structural components being permanently engaged with the casing to provide electrical insulation.

The first and second structural components having annular cross-sections.

The first and second structural components have annular cross-sections with an inner diameter larger than the diameter of the proximal and distal insulated portions of the conductive element and preferably an outer diameter essentially same as the inner diameter of casing.

At least part of the non-insulated portion of the conductive element being disposed in an inner casing of an electrically conductive, non-degradable material, the inner casing having an outer diameter which is equal to or smaller than the inner diameter of the casing.

The center of the first and second structural components being linearly aligned along the main axis.

Some embodiments (denoted E) comprise the following features in addition to the general features:
An elongated conductive element having a proximal and distal end, comprising insulated proximal and distal portions, said proximal insulated portion extending in distal direction from the proximal end, said distal insulated portion extending in proximal direction from the distal end, the proximal and distal portions separated by a non-insulated portion of the conductive element.
The at least non-insulated portion of the conductive element being essentially centrally disposed in a casing of an electrically insulating non-degradable material by a first and a second structural component, said components extending in radial direction between the casing and the conductive element, said first structural component being movably disposed around the proximal insulated portion, said second structural component being disposed around the distal insulated portion.
The casing, first and second structural components forming a distal chamber encapsulating the non-insulated portion of the conductive element.
The casing having an opening distally to the second structural component.
The casing configured to electrically couple the non-insulated portion of the conductive element with the soft tissue.

Any and any number of the below features may be combined with the above embodiment E features:

At least part of the non-insulated portion of the conductive element being disposed in an inner casing of an electrically conductive, preferably non-degradable material, the inner casing having an outer diameter which is equal to or smaller than the inner diameter of the casing.

The first and second structural components not forming part of the casing.

The first and second structural components being permanently engaged with the casing to provide electrical insulation.

The inner casing having an annular cross-section having an inner diameter equal to or larger than the diameter of the insulated proximal and distal portions. The first and second structural components having annular cross-sections.

The first and second structural components having annular cross-sections with an inner diameter larger that the diameter of the proximal and distal insulated portions of the conductive element and preferably an outer diameter essentially same as inner diameter of casing.

### Presentation of Some Embodiments in More Detail

The invention encompasses for instance the following embodiments:

A microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope, sheath) of electrically insulating non-degradable material, wherein the non-insulated portion of the element is encapsulated (surrounded) by the casing forming a distal chamber, in which the conductive element can slide in an axial direction, the casing of the distal chamber comprising electrically conductive bridges electrically coupling the distal chamber with the adjacent soft tissue, and wherein the casing comprises a first structural component in which the electrically insulated portion of the conductive element can slide in an axial direction.

A microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope, sheath) of electrically insulating non-degradable material, the casing comprising a first structural component, which first structural component may form part of the casing, wherein the non-insulated portion of the conductive element is encapsulated (surrounded) by the casing forming a distal chamber, the first structural component being detached from the conductive element, wherein the casing of the distal chamber comprises at least one conductive bridge electrically coupling the distal chamber with the adjacent soft tissue.

A microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulted portion, at least part of the conductive element being disposed in a casing (envelope) of electrically insulating non-degradable material, the casing comprising a first structural component which first structural component may form part of the casing, the non-insulated portion of the conductive element being encapsulated by the casing and the first structural component forming a distal chamber, the casing and the first structural component essentially partitioning the non-insulated portion of the conductive element from the adjacent soft tissue, the first structural component being detached from the conductive element, wherein the distal chamber comprises electrically conductive bridges electrically coupling the distal chamber with the adjacent soft tissue.

A microelectrode configured to be at least partially embedded into or at least partially placed adjacent to soft tissue, in particular nervous, endocrine and muscle tissue, comprising an oblong (elongated) electrically conductive element, the electrically conductive element comprising a proximal electrically insulated portion and distal non-insulated portion, at least part of the conductive element being disposed in a casing (envelope, sleeve, sheath) comprising [of] electrically insulating non-degradable material wherein the non-insulated portion of the element is encapsulated (surrounded) by the casing forming a distal chamber the casing of the distal chamber comprising at least one electrically conductive bridge electrically connecting the distal chamber with the adjacent soft tissue (fluid) selected from any one of a) hole in the casing, b) filament-like structures penetrating the casing of the distal chamber, c) a lateral member forming part of the casing of the distal chamber, the lateral member selected from any one of a sheet comprising a conductive material and an ion-permeable membrane (sheet-like structure) admitting the transfer of ions between the distal chamber and the adjacent soft tissue forming part or the distal chamber, and wherein the casing comprises a first structural component slidably mounted [engaged] around at least part of the electrically insulated portion of the conductive element, the first structural component enabling the casing to slide in an axial direction with respect to the electrically conductive element.

A microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by a least a first structural component which may form part of the casing, the first structural component extending in radial direction between the casing and the conductive element, the casing and the first structural component forming a distal chamber around the non-insulated portion of the conductive element, the casing and the first structural component essentially partitioning the distal chamber from the adjacent soft tissue, wherein either the first structural component is detached from the conductive element, or the casing is detached from the first structural component and wherein the casing comprises at least one electrically conductive bridge, the bridge configured to electrically connecting the chamber with adjacent soft tissue.

A microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by at least a first structural component which may form part of the casing, the first structural component extending in radial direction between the casing and the conductive element, the casing and the first structural component forming a distal chamber around the non-insulated portion of the conductive element, the casing and the first structural component essentially partitioning the distal chamber from the adjacent soft tissue, the casing being movably associated with the conductive element, wherein the casing comprises at least one electrically conductive bridge, the bridge configured to electrically connecting the distal chamber with adjacent soft tissue.

A microelectrode configured to be at least partially embedded into or at least partially placed adjacent to soft tissue, in particular nervous, endocrine and muscle tissue, comprising an elongated electrically conductive element having a proximal and distal end, the electrically conductive element comprising insulated proximal and distal portions, the proximal insulated portion extending in distal direction form the proximal end, the distal insulated portion extending in proximal direction from the distal end, the proximal and distal portions separated by a non-insulated portion of the conductive element, at least the non-insulated portion of the conductive element being essentially centrally disposed in a casing of an electrically insulating non-degradable material, the microelectrode further comprising first and second structural components, said first and second structural components extending in radial direction between the casing and the conductive element, the first structural component movably disposed around the proximal insulated portion, the second structural component movably disposed around the distal insulated portion; the casing, first and second structural components forming a distal chamber, wherein the casing has an opening distally to the second structural component, wherein the casing is configured to electrically couple the non-insulated portion of the conductive element with the soft tissue.

A microelectrode configured to be at least partially embedded into or at least partially placed adjacent to soft tissue, in particular nervous, endocrine and muscle tissue, comprising a conductive element comprising a distal and a proximal non-insulated portion and an insulated portion between the distal and proximal non-insulated portions, at least the proximal non-insulated portion of the conductive element being essentially centrally disposed in a casing of an electrically insulating non-degradable material, the microelectrode further comprising a first structural component which may form part of the casing, the first structural component being movably disposed around the insulated portion of the conductive element, the casing and the first structural component essentially electrically insulating the proximal non-insulated portion the soft tissue, the inside of the casing comprising electrically conductive material electrically coupling the proximal non-insulated section of the conductive element, the electrically conductive material in electrical communication with an insulated lead attached to the proximal end of the casing.

### Disclosure of Certain Features in More Detail

### Electrical Coupling

The casing of the microelectrode enclosing a non-insulated portion of the conductive element is configured to electrically couple a non-insulated portion of the conductive element with the soft tissue. A non-insulated conductive element within a distal chamber enclosed by a casing and at least one structural component electrically interacts with adjacent soft tissue by an electrical coupling/electric bridge based on one or both of: a) the exchange of charged particle across the casing, herein referred to as a fluidic electrically conductive bridge, or, b) electrically connecting the enclosed non-insulated portion of the conductive element with the soft tissue without the exchange of charged particles (ions and electrons) across the casing, herein referred to as an electric conductive bridge.

An electric conductive bridge may also exhibit the capability of exchanging charged particles across the casing

For the non-insulated portion of the conductive element to electrically communicate via an electrical coupling comprised in the casing the distal compartment comprises at least one conductive medium. Such media may be selected from electrolytic aqueous solutions and/or conductive gels like conductive hydrogels. According to an aspect, the medium may be a combination of an electrolytic aqueous solution and a conductive gel. The conductive medium may already be comprised within the distal chamber prior to insertion. Alternatively, the conductive medium can be formed upon insertion into soft tissue by the diffusion of soft tissue fluids inside the distal chamber. Soft tissue fluid may diffuse into the distal chamber through the lumen/void/gap between the first and optional second structural components between the structural component and an insulated portion of the conductive element or between the structural components and the casing (depending on the specific embodiment). Soft tissue fluids may also diffuse into the distal chamber trough fluidic conductive bridges.

The design of the electrical coupling of the casing is inter alia governed on the application of the microelectrode. If high specificity and/or resolution is needed the total surface area of the conductive bridge or bridges is limited. A limited area of the electric coupling is e.g. useful for monitoring or stimulating individual or a limited number of neurons in nervous tissue.

For other applications it is useful that electrical coupling covers multiple areas of the casing such as multiple conductive bridges. Said combined number of conductive bridges may cover a substantial area of the casing. Alternatively, the electrical coupling covers a substantial area of the casing by a single conductive bridge.

Electric coupling of the casing can be obtained by a fluidic electrically conductive bridge and/or an electric conductive bridge.

An electric conductive bridge may also comprise a fluidic electrically conductive bridge.

Certain fluidic electrically conductive brides do not include a non-fluidic electrically conductive bridge.

Some embodiments comprise at least an electric conductive bridge and may further comprise at least one fluidic electrically conductive bridge.

Some embodiments comprise any type of electrical coupling disclosed herein.

The electrical coupling/bridge is typically disposed laterally with respect to the distal chamber (laterally to the main axis of the conductive element).

Electrically conductive bridges may be selected from hollow conductive elements, conductive filament-like structures penetrating the casing of the distal chamber, lateral members, lateral sheet-like members, lateral, sheet-like, conductive members, conductive sheet-lime structures, and conductive, non-degradable ion-permeable membranes.

The electrically conductive bridges are preferably non-degradable in tissue fluids.

Electrically conductive filament-like structure penetrating the distal casing may be selected from hollow conductive filament-like structures (a filament-like comprising a central conduit). A conductive, hollow filament-like structure is representative for an electrically conductive bridge comprising a fluidic electrically conductive bridge. A non-conductive hollow filament-like structure is only capable of electrically coupling the non-insulated portion of a conductive element with the adjacent soft tissue by exchanging charged particle across the casing, hence, falling under the definition of a fluidic electrically conductive bridge. The conductive filament-like structure may also be solid.

An electrically conductive bridges may comprise metals, metal alloys and/or conductive polymers and/or carbon-containing materials such as graphene, graphite, and carbon nanotubes. According to a further aspect, an electrically conductive bridges may be configured such that they exhibit a significant surface area of the parts in contact with fluids in the distal chamber and in contact with adjacent soft tissue. Electrically conductive bridges configured with high surface areas contacting the adjacent soft tissue and fluid of the distal chamber makes the information transmission more effective between microelectrode and adjacent soft tissue.

A conductive filament-like electrically conductive bridge is preferably disposed with respect to the casing that the angle of an electrically conductive filament with respect to the normal of the casing surface is more than 45°, suitably more than 70°. Preferably, the angle of average trajectory of a majority of electrically conductive bridges is not lower than 60°, not lower than 70°.

Fluidic electrical bridges may be an opening, preferably a lateral opening in the distal casing. An opening may an area of about 1 μm² or more. Preferably, the area of an opening up to the combined area of all openings of the casing of the distal chamber may range from about 1 μm² of an opening up to a combined area of all openings of about 150000 μm² or more. An opening is preferably configured to prohibit the blockage of the opening by tissue growth. It has been observed that glial cells can cover small opening and then to some extent isolate the interior of the distal chamber from the surrounding neurons. A preferred range of the area of an opening is from about 20 μm² up to about 2000 μm², suitably from about 100 to about 1500 μm².

According to a further aspect, the casing of the distal chamber comprises a plurality of openings in the distal casing. According to yet a further aspect, the maximum number of openings of the distal chamber is given by the maximum number of openings not significantly compromising the structural rigidity/conformation of the distal casing.

The number of openings depends to a degree on the volume of the distal chamber, type of material(s) of the casing and the mode of operation of the microelectrode. When using the microelectrode for stimulation of soft tissue it may be preferable to have a higher total area of openings (higher number of openings) than when using the microelectrode for soft tissue monitoring purposes. Should the microelectrode operate both in stimulation and monitoring mode the total area of openings (number of openings) should preferably be within a range satisfying both the needs of stimulation and monitoring modes. The upper number of openings is to an extent governed by the structural rigidity of the distal chamber (of the casing encapsulating the distal chamber), the total area of one opening up to the combined total area of all openings may range of from about 20 μm² up to about 150000 μm² or more.

An electrically conductive bridge may be a lateral member, such as a lateral, sheet-like, non-degradable conductive member, non-degradable conductive sheet-lime structure, and conductive, non-degradable ion-permeable permeable membrane, forming part of the casing of the distal chamber. By lateral member forming part of the casing is meant that at least a section of the casing is replaced by a lateral member. Hence, the lateral member typically maintains the three-dimensional shape of the distal casing. The lateral member has an overall sheet-like appearance. The lateral member electrically connects the distal chamber with the adjacent soft tissue. This electrically coupling/connection may be attained by an electrically conductive material and optionally additionally by using a lateral member allowing transport of electrically conductive particles, notably ions and electrons. Thus, the lateral member may be selected from conductive sheet-formed members comprising a conductive material and/or an ion-permeable membrane.

An ion-permeable membrane should be understood as a material or configuration enabling the transport of charged particles such as ions. The ion-permeable membrane may be made of a porous polymeric material. The polymeric material may be non-conductive or conductive. The ion-permeable material may be configured like a mesh, net or web. The mesh, net or web may comprise rods/threads/filaments. The rods/threads/filament may be conductive. The lateral member may be an ion-permeable material comprising conductive rods/threads/filaments.

The lateral member is preferably configured to reduce or eliminate tissue from entering the distal chamber. One way of reducing the ability of tissue to entering the distal chamber is to configure the dimension (area) of each mass-transfer channel that the risk of tissue entering the distal chamber is reduced or eliminated.

A further measure for reducing or eliminating tissue from entering the distal chamber is the provision of an inner casing preferably having an annular shape with an outer diameter essentially matching the inner diameter of the casing, further preferably having an inner diameter which is larger than the diameters of the insulated proximal/distal portions of the conductive element

The lateral member (ion-permeable membrane) may be selected from any one of a porous polymeric material, mesh, net, web.

The lateral member may be selected from a mesh, net, web comprising a conductive material.

The microelectrode where at least part of the casing of the distal chamber constitutes of a lateral member is specifically suited for the stimulation of soft tissue, such as nervous tissue.

The area of the lateral member based on the total area of the casing of the distal chamber which is essentially parallel to the main axis of the elongated conductive element is from about 10%, from about 15%, from about 20% up to about 50%, up to about 60%, up to about 70%, up to about 80%, up to about 90%, up to 100%.

The lateral member has suitably a radial extension of up to about 30°, up to about 60°, up to about 90°, up to about 180°, and up to about 360°.

In some embodiments, the casing of the distal chamber comprises electrically conductive bridges electrically coupling the distal chamber with the adjacent soft tissue.

In some embodiments, the distal casing comprises at least one electrically conductive bridge electrically connecting the distal chamber with the adjacent soft tissue (fluid) selected from any one of a) an opening in the casing, b) a filament-like structure penetrating the casing of the distal chamber, c) a lateral member forming part of the casing of the distal chamber.

In some embodiments, the distal casing comprises at least one electrically conductive bridge electrically connecting the distal chamber with the adjacent soft tissue (fluid) selected from any one of a) a filament-like structure penetrating the casing of the distal chamber, b) a lateral member forming part of the casing of the distal chamber.

### Conductive Element

All aspects of the invention comprise an electrically conductive element. The electrically conductive element can be referred to as an oblong, electrically conductive element or an elongated, electrically conductive element. The electrically conductive element may also be referred to as a filament, such as a thin, fine, elongated filament. The electrically conductive element may be understood as a thin electrically conductive filament, typically rotationally symmetric, with an axial extension significantly larger that the radial extension. The diameter or thickness in the range of from about a few μm, e.g. 2 μm, up to about 100 μm. The elongated electrically conductive element (including non-insulated and insulated electrically conductive element) typically has a length of from about 2 mm up to about 1 m. The non-insulated portion of the conductive element may be roughened or e.g. comprise nano- or micro-sized granules to reduce impedance. The casing of the microelectrode has typically an elongated form having an axial extension from about 50 μm up to about 20 mm or more, suitably from about 500 μm up to about 15 mm but can be longer. The elongated electrically conductive element may comprise several sub-portions such as non-insulated and insulated portions. An electrically conductive element may be composed of a plurality of micro or nano wires which are electrically connected. The conductive element may be a single rod, homogeneous rod or be designed as a multiwire element or multifilament (element), for example a twisted multiwire. A multiwire electrode element usually has a larger surface area than that of a single wire element of the same diameter and thus a lower impedance.

According to an embodiment, the conductive element comprises a central conduit which may be used for the delivery of pharmacologically potent fluids (drugs) to the distal chamber and through fluidic electrically conductive bridges comprised in the casing of the distal chamber.

The conductive element may also be hollow, i.e. comprising a central conduit. A conductive element with a central conduit can be used for the delivery of various biologically active substances to soft tissue adjacent the microelectrode.

### First and Optional Second Structural Components

First and second structural components may form part of the casing. Preferably, first and second structural components are separate from the casing. First and second structural components preferably have an annular cross-section. First and second structural components preferably extends in radial direction between the casing and the any insulated portion of the conductive element. First and second structural components essentially centrally dispose the conductive element with respect to the casing.

First and second structural components are preferably made of electrically insulating, non-degradable materials.

### Casing

The casing is made of an electrically insulating non-degradable material. Preferably, the casing is flexible. The casing may comprise a flexible and soft polymeric material, such as Teflon, silicon or parylene C.

The casing may be configured such that the volume of the casing can vary. The movement of the casing with respect to the conductive element (and the insulated section of the conductive element) may influence the pressure inside the casing. The casing is preferably configured to adapt to pressure variance caused by the mutual movement of the conductive element and casing thereby counteracting the pressure variances. If the mutual movement temporarily increases the pressure inside the casing the volume of the casing increases. If the mutual movement temporarily decreases the pressure inside the casing the volume of the casing decreases.

In some embodiments the conductive element in disposed in a casing, the casing comprising at least a first structural component partitioning the casing into a distal chamber and proximal compartment. The terms chamber and compartment have been chosen partly for added clarity. Additionally, the words ‘chamber’ and ‘compartment’ to an extent serve different purposes and more importantly, the distal chamber embraces/encapsulates the non-insulated portion of the conductive element while the insulated portion of the conductive element is disposed mainly or at least partly in the proximal compartment. For some embodiments the casing, first and second structural components forms a distal chamber.

The casing, or at least part of the casing, has preferably a rotationally symmetric shape, suitably cylindrical shape. Preferably the radial extension (diameter) of the casing of the distal chamber and at least part of the casing of the proximal compartment (typically the distal portion of the proximal compartment) is similar or essentially same. Suitably, the radial extension over the distal chamber and at least part of the proximal compartment does not differ more than 20%, typically not more than 10%.

Suitably, the distal section of the casing narrows down preferably in a form facilitating insertion into soft tissue. The cross-section of the distal section of the casing preferably exhibits a convex line segment.

In some embodiments comprising first and second structural components, the part of the casing distally to the second structural components comprises an opening, said opening preferably being symmetrical to the main axis of the microelectrode.

According to yet a further aspect, the diameter of the proximal compartment widens in a proximal direction.

According to some embodiments the first structural component partitions the casing thereby forming a distal chamber and a proximal compartment. Hence, the casing restricts part or essentially all the volume of the distal chamber and proximal compartment.

### Inner Casing

It has been noted that a certain instability in recordings can occur when using microelectrodes disclosed in WO 2022005386A1. A likely reason is that glial cells and extracellular matrix may anchor to the conductive element through the opening in the casing partly supported by remnants of tissue inside the casing in scientific studies. Such an anchoring likely causes the tissue adjacent to the opening to move into or out from the casing as the conductive element is moving, which in turn will affect the amplitude of recorded neuronal signals. This may also cause irritation of nearby tissue from which neuronal recordings are made.

To further prevent a direct physical contact between tissue and the conductive element it is preferred to implement an electrically conductive, non-degradable inner casing around at least part of the non-insulated portion of the conductive element in the distal compartment in which the insulated conductive element can slide. The lumen of the inner casing needs to be larger than the diameter of the insulated conductive element.

An inner casing may be deployed in any one of the microelectrodes disclosed herein.

An inner casing is particularly preferred for microelectrodes comprising a fluidic electrically conductive bridge such as an opening in the casing. The presence of an inner casing prevents tissue from entering the distal compartment or prevents tissue from attaching to the non-insulated portion of the conductive element should the inner casing engage with the inside of the casing.

Thus, in some embodiments, the non-insulated portion of the conductive element in the distal chamber is disposed in an inner casing comprising of an electrically conductive, non-degradable material. The material of the inner casing can be selected from any conductive material as long as such material substantially inhibits tissue to attach to the non-insulated portion of the conductive element. The material may be selected from conductive gel-like materials and non-gel materials comprising a porosity essentially inhibiting tissue from penetration and other types of biocompatible cross-linked matrix materials with electrically conductive properties. A preferred material of the inner casing are hydrogels. Hydrogels are crosslinked, non-water dissolvable hydrophilic polymers, typically forming three-dimensional network structures. Hydrogels are highly absorbent while essentially maintaining well-defined spatial structures. Chemical and physical hydrogels may be employed. While chemical hydrogels comprise covalent cross-linking bonds, the integrity of physical hydrogels is based on non-covalent intra-molecular attractions between polymers like hydrogen bonds, hydrophobic interactions, and polymeric chain entanglement. Hydrogels can be prepared by a variety of natural and synthetic polymers. Natural polymers for hydrogel preparation, the preparation usually encompassing cross-linking, include peptides, collagen, gelatine, cellulose, hyaluronic acid, chitosan, heparin, alginate, Pedot and fibrin to mention a few. Common synthetic polymers include polyvinyl alcohol, polyethylene glycol, sodium polyacrylate, acrylate polymers and copolymers thereof. Acrylate-siloxane hydrogels may also be applied. The mechanical properties of hydrogels may preferably be modulated by the degree of cross-linking.

The inner diameter of the inner casing is preferably larger than the diameter of the insulated conductive element to not hinder axial movements of the conductive element. The conductive inner casing is placed between the first structural component and the optional second structural component. The inner casing is surrounded by the casing. This arrangement is based on the insight that it is a significant advantage to prevent tissue to accumulate inside the distal chamber since tissue matrix materials can adhere to the conductive element and thereby propagate movements of the conductive element to the tissue outside the contact and thereby cause positional instability of the tissue relative to the contact.

### Embodiments D and E

These embodiments further reduces changes in volume/pressure of body fluid inside the distal chamber as the conductive element is moving in an axial direction. Such changes of volume/pressure may otherwise cause a resistance to axial movements of the conductive element and fluidic movements through the contact (in case the contact comprise openings) that in turn can cause positional instability of the tissue adjacent to the contact. It also serves to inhibit tissue from growing into the distal chamber.

This embodiment relates to a microelectrode configured to be at least partially embedded into or at least partially placed adjacent to soft tissue, in particular nervous, endocrine and muscle tissue, comprising an elongated electrically conductive element comprising a proximal and distal end, the electrically conductive element comprising insulated proximal and distal portions, the proximal insulated portion extending in distal direction from the proximal end, the distal insulated portion extending in proximal direction from the distal end, the proximal and distal portions separated by a non-insulated portion of the conductive element, at least the non-insulated portion of the conductive element being essentially centrally disposed in a casing of an electrically insulating non-degradable material, the microelectrode further comprising first and second structural components, said first and second structural components extending in radial direction between the casing and the conductive element, the first structural component movably disposed around the proximal insulated portion, the second structural component movably disposed around the distal insulated portion; the casing, first and second structural components forming a distal chamber, wherein the casing has an opening distally to the second structural component, wherein the casing is configured to electrically couple the non-insulated portion of the conductive element with the soft tissue.

Preferably, at least part of the non-insulated portion of the conductive element is disposed in an inner casing of an electrically conductive, preferably non-degradable material, the inner casing having an outer diameter which is equal to or smaller than the inner diameter of the distal casing. The inner casing preferably has an annular cross-section having an inner diameter equal to or larger than any of the diameters of the insulated proximal and distal portions of the conductive element. Preferably, the non-insulated portion of the conductive element is disposed within the inner casing to the extent tissue inside the distal chamber is not capable to attach to the non-insulated portion of the conductive element. Preferably, the entire non-insulated portion of the conductive element is disposed within the inner casing. The inner casing may also be movably disposed around part of the proximal insulated portion and/or around part of the distal insulated portion of the conductive element.

The inner casing comprises an electrically conductive, preferably non-degradable, material.

First and second structural components are preferably separate entities, preferably having annular cross-sections, extending radially between the casing and the insulated distal and proximal portions of the conductive element. Preferably, the first and second structural components have annular cross-sections where the inner diameters are larger than the diameters of the insulated proximal and distal portions.

First and second structural components are preferably permanently attached to the casing, preferably electrically sealing first and second structural components with the casing thereby prohibiting electrical currents between said first and optional second structural components and the casing.

The electrical coupling of the casing of the distal chamber can be any of the electrical couplings disclosed herein.

According to an aspect, the diameter of the distal insulated portion of the conductive element is larger than the inner diameter of the first structural component and possibly also larger than the diameter of the proximal insulated conductive element. When a microelectrode is withdrawn from the soft tissue the distal insulated portion is prohibited from sliding though the first structural component. As the casing is attached to the first and second structural component the complete microelectrode is withdrawn from the soft tissue by pulling the conductive element or any lead attached to the conductive element.

An alternative solution/aspect is that the distal insulated portion comprises a protrusion positioned distally to the second structural component prohibiting the distal insulated portion of the conductive element to completely slide through the second structural component when the conductive element pulled in proximal direction.

### Further Disclosure of Embodiments

One embodiment of the invention is specifically configured for electrically stimulating soft tissue, the embodiment comprising a casing delivering electric current through a tissue volume adjacent the microelectrode, the casing being fully detached from the conductive element providing the electric current. With this configuration efficient electric stimulation is provided to same or essentially the same part of the soft tissue. A microelectrode for soft tissue stimulation purposes preferably has an electrically conductive bridge selected from lateral members like lateral sheet-like members.

An advantage of the invention is that direct physical contact of a conductive electrode (herein referred to as conductive element) of an implanted microelectrode with adjacent soft tissue, in particular nervous tissue but also endocrine tissue, exocrine tissue, muscular tissue, heart tissue, connective tissue and retina, can be avoided by encapsulating the conductive element with a casing of an electrically insulating non-degradable material.

In some embodiments, the casing is associated with the conductive element such that the distal tip of the non-insulated portion of the conductive element does not touch nor penetrate the casing of the distal chamber. This can be useful in situations with small tissue movements relative to the lead.

In some embodiments, the elongated electrically conductive element comprises a proximal and distal end, the electrically conductive element comprising insulated proximal and distal portions, the proximal insulated portion extending in distal direction from the proximal end, the distal insulated portion extending in proximal direction from the distal end, the proximal and distal portions separated by a non-insulated portion of the conductive element. The non-insulated portion of the conductive element is disposed in a casing of an electrically insulating non-degradable material, the conductive element being preferably essentially centrally disposed in the casing by at least a first structural component which may form part of the casing, the casing and the first structural component forming a distal chamber around the non-insulated portion of the conductive element, the insulated distal portion of the conductive element protruding distally from the distal end of the casing, the distal end of the casing preferably acting as a second structural component.

Thus, preferably not only a proximal portion of the conductive element is insulated but also a distal portion of the conductive element is insulated distally to the distal non-insulated portion of the conductive element (referred to as distal insulated portion of the conductive element). The distal insulated portion of the conductive element may be disposed inside the distal chamber, however, the distal insulated portion of the conductive element may also be disposed such that this portion penetrates the distal end of the casing. Alternatively, the insulated distal portion of the conductive element protrudes through an opening in the distal end of the casing, the opening in the distal end of the casing acting as radially stabilizing the distal insulated portion of the conductive element. In the latter configuration, the casing of the distal chamber may function to stabilize the distal region of the conductive element in radial direction. The casing, or the distal end of the casing assumes the function of a second structural component.

The first and optional second structural components may also be distinct from the casing and exhibit a tubular structure, preferably with an annular cross-section, in which the insulated conductive portions of the conductive element can slide. The first and optional second structural components are preferably made of a non-degradable electrically insulating material, for example Teflon or Nylon, that ensures a sufficiently low friction against the insulated conductive element. The detachment of the conductive element from the protective casing and the potential adherence of the casing to the surrounding soft tissue enables the casing to a (n) (significant) extent accommodate to any movement of the surrounding tissue while preserving that the electrode monitors and/or stimulates essentially the same region of the soft tissue over time.

The detachment of the casing from the conductive element (or vice-versa) enables the soft tissue surrounding the microelectrode to move without significantly influencing/altering the perpendicular distance (FIG. 24, 104) of the non-insulated distal portion of the element to the electrical coupling of the casing, e.g. by conductive bridges, since the perpendicular distance to the non-insulated portion of the conductive element will not change substantially when the non-insulating portion of the element slide in an axial direction. A radially stabilized non-insulated portion of the conductive element improves the recorded signal pattern (fingerprint) and improves the efficacy of stimulation of targeted excitable cells in the case where the microelectrode is used for stimulation. An optional second structural component inside the distal chamber further stabilizes the non-insulated distal portion in radial direction providing that the perpendicular distance (FIG. 24, 104) between the non-insulated distal portion of the element and the conductive bridges remain essentially the same over time.

The first and second structural components promote that that the nearest distance to the non-insulated portion of the conductive element from the electrical bridge remains essentially the same.

A still further advantage of the present invention is that the casing once inserted into soft tissue may adhere to the soft tissue in a way that minimizes or even essentially prohibits movement of the casing vis-à-vis the surrounding soft tissue. When the soft tissue moves the casing moves with the soft tissue. The decoupling of the casing from the conductive element inside the casing is an important feature of the invention for minimizing or essentially prohibiting the movement of the casing in relation to the surrounding soft tissue.

Some embodiments of the microelectrode of the present invention preferably provides a high surface area of the non-insulated conductive element while simultaneously providing the stimulation and monitoring of a spatially specific region of the soft tissue. A high surface area for example a roughened surface of the non-insulated conductive element may be provided by any surface enlarging surface modification for example by laser milling.

The invention relates, inter alia, to a microelectrode, a microelectrode probe, a microelectrode array and a method for producing a microelectrode, microelectrode probe and an array of microelectrodes. The microelectrode probe constitutes a version of the microelectrode which is designed to be inserted into soft tissue. Hence, the microelectrode probe comprises certain components providing the probe with sufficient rigidity to enable successful insertion into various soft tissues. Once inserted into soft tissue, certain components of the microelectrode probe dissolves and/or disintegrates upon contact with body fluids transforming the microelectrode or microelectrode array gradually into the microelectrode, an in-situ microelectrode.

Common to all aspects of the invention is the microelectrode/microelectrode probe to be at least partially embedded or inserted into soft tissue or at least partially or entirely placed adjacent to soft tissue.

Common to all aspects of the invention (microelectrode, proto-microelectrode, microelectrode probes, arrays) is that the casing of the distal chamber is configured to electrically couple the non-insulated portion of the conductive element with the soft tissue, e.g. by at least one conductive bridge.

The microelectrode may be implanted in soft tissue or positioned adjacent to the surface of target soft tissue. By adjacent should be understood that at least part of the microelectrode is not surrounded by target soft tissue. Certain soft tissues may preferably be monitored and/or stimulated by the microelectrode by an adjacent positioning with respect to the soft tissue. Spinal nervous tissue, peripheral nerves, dorsal root ganglia, retina and sensory tissue such as auditory may advantageously be monitored and/or stimulated by positioning the microelectrode or microelectrode arrays adjacent to the nervous tissue.

The part of the casing forming the proximal compartment may also be denoted proximal casing or casing of the proximal compartment, the part of the casing forming the distal chamber may also be denoted distal casing or casing of the distal chamber.

The term microelectrode as used herein includes at least a conductive element and a casing as described in any of the aspects/embodiments such as comprising at least a first structural component and further comprising at least one conductive bridge (in the part of the casing of the distal chamber.

In some embodiments the conductive element in disposed in a casing, the casing comprising a first structural component partitioning the casing into a distal chamber and proximal compartment. The terms chamber and compartment have been chosen partly for added clarity. Additionally, the words ‘chamber’ and ‘compartment’ to an extent serve different purposes and more importantly, the distal chamber embraces in essence the non-insulated portion of the conductive element while the insulated portion of the conductive element is disposed mainly or at least partly in the proximal compartment.

The microelectrode or microelectrode probe disclosed herein may also comprise a proximal bridging arrangement associated with the proximal region of the insulated section of the conductive element This proximal bridging arrangement is configured to be slidably associated with the proximal region of the insulated section of the conductive element. The proximal bridging arrangement comprises a casing exposing a conductive layer, said casing encapsulating a proximal non-insulated section of the conductive element proximally to the insulated section of the conductive element. The proximal non-insulated section of the conductive element located proximally to the insulated section of the conductive element is not in direct contact with the surrounding soft tissue. Thus, body fluid inside the casing of the proximal bridging arrangement provides the conductive bridge between the proximal non-insulated section of the conductive element proximally to the insulated section of the conductive element and the conductive layer of the casing.

Some embodiments relate to a microelectrode (or proximal bridging arrangement) comprising a conductive element comprising distal and proximal non-insulated sections and further an insulated section between said distal and proximal non-insulated section, where the insulated section of the conductive element is slidably associated with a casing encapsulating the proximal non-insulated section of the conductive element, the casing further insulating the proximal non-insulated section of the conductive element from direct contact with adjacent soft tissue once the microelectrode is inserted into soft tissue, the casing exposing conductive material capable of electrically coupling the proximal non-insulated section of the conductive element with a second conductive element electrically coupled to the conductive material of the casing.

Additional embodiments relate to a microelectrode configured to be at least partially embedded into or at least partially placed adjacent to soft tissue, in particular nervous, endocrine and muscle tissue, comprising a conductive element comprising a distal and a proximal non-insulated portion and an insulated portion between the distal and proximal non-insulated portions, at least the proximal non-insulated portion of the conductive element being essentially centrally disposed in a casing of an electrically insulating non-degradable material, the microelectrode further comprising a first structural component which may form part of the casing, the first structural component being movably disposed around the insulated portion of the conductive element, the casing and the first structural component essentially electrically insulating the proximal non-insulated portion the soft tissue, the inside of the casing comprising electrically conductive material electrically coupling the proximal non-insulated section of the conductive element, the electrically conductive material in electrical communication with an insulated lead attached to the proximal end of the casing.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by at least a first structural component which may form part of the casing, the first structural component extending in radial direction between the casing and the conductive element, the casing and the first structural component forming a distal chamber around the non-insulated portion of the conductive element, the casing and the first structural component essentially partitioning/restricting the distal chamber from the adjacent soft tissue, the casing being movably associated with the conductive element, wherein the casing comprises at least one electrically conductive bridge, the bridge configured to electrically connecting the distal chamber with adjacent soft tissue.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by a first structural component which may form part of the casing, the first structural component extending in radial direction between the casing and the conductive element, the casing and the first structural component forming a distal chamber around the non-insulated portion of the conductive element, the casing and the first structural component essentially partitioning/restructing the distal chamber from the adjacent soft tissue, wherein either the first structural component is detached from the conductive element, or the casing is detached from the first structural component and wherein the casing comprises at least one electrically conductive bridge, the bridge configured to electrically connecting the chamber with adjacent soft tissue.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulted portion, at least part of the conductive element being disposed in a casing (envelope) of electrically insulating non-degradable material, the casing comprising a first structural component which first structural component may form part of the casing, the non-insulated portion of the conductive element being encapsulated by the casing and the first structural component forming a distal chamber, the casing and the first structural component essentially partitioning/restricting the non-insulated portion of the conductive element from the adjacent soft tissue, the first structural component being detached from the conductive element, wherein the distal chamber comprises electrically conductive bridges electrically coupling the distal chamber with the adjacent soft tissue.

Some embodiments relate to a microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope) of electrically insulating non-degradable material the casing comprising a first structural component, which first structural component may form part of the casing, wherein the non-insulated portion of the conductive element is encapsulated (surrounded) by the casing forming a distal chamber, the first structural component being detached from the conductive element, wherein the casing of the distal chamber comprises at least one conductive bridge electrically coupling the distal chamber with the adjacent soft tissue.

Some embodiments relate to a microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber comprising electrically conductive bridges electrically coupling the distal chamber with the adjacent soft tissue, and wherein the casing comprises a first structural component in which the electrically insulated portion of the conductive element can slide in an axial direction.

One embodiment of the invention relates to a microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion, a distal non-insulated portion and optionally a distal insulated portion distally to the non-insulated portion, at least part of the conductive element being disposed in a casing (envelope) comprising of electrically insulating non-degradable material, wherein the non-insulated portion of the element is encapsulated (surrounded) by the casing a first and optional second structural components, said casing, first and optional second structural components forming a distal chamber, in which casing the conductive element can slide in an axial direction, the casing optionally comprising an opening distally to the second structural component, the casing of the distal chamber comprising electrically conductive brides selected from selected from any one of a) a filament-like structure penetrating the casing of the distal chamber, b) a lateral member forming part of the casing of the distal chamber.

A further embodiment of the invention relates to a microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion and distal non-insulated portion, at least part of the conductive element being disposed in a casing (envelope) comprising of electrically insulating non-degradable material, wherein the non-insulated portion of the element is encapsulated (surrounded) by the casing forming a distal chamber, in which the conductive element can slide in an axial direction, the casing of the distal chamber comprising at least one electrically conductive bridge, such as an electrically conductive bridge comprising at least one non-fluidic electrically conductive bridge/element connecting the distal chamber with the adjacent soft tissue selected filament-like structures penetrating the casing of the distal chamber, wherein the casing comprises a first structural component slidably mounted (engaged) around the electrically insulated portion of the conductive element, the first structural component enabling the casing to slide in an axial direction with respect to the conductive element.

A further embodiment relates to a microelectrode configured to be at least partially embedded into or at least partially placed adjacent to soft tissue, in particular nervous, endocrine and muscle tissue, comprising an elongated electrically conductive element, the elongated electrically conductive element comprising a proximal electrically insulated portion and distal non-insulated portion, at least part of the conductive element being disposed in a casing (envelope) comprising of electrically insulating non-degradable material, wherein the non-insulated portion of the element is encapsulated (surrounded) by the casing forming a distal chamber, in which the conductive element can slide in an axial direction, the casing of the distal chamber comprising at least one electrically conductive bridge, such as an electrically conductive bridge comprising at least one non-fluidic electrically conductive bridge/element, electrically connecting the distal chamber with the adjacent soft tissue selected from a lateral member forming part of the casing of the distal chamber selected from any one of a sheet comprising a conductive material and an ion-permeable membrane (in form of a sheet-like structure) admitting the transfer of ions between the distal chamber and the adjacent soft tissue forming part or the distal chamber rand wherein the casing comprises a first structural component slidably mounted [engaged] around the electrically insulated portion of the conductive element, the first structural component enabling the casing to slide in an axial direction with respect to the electrically conductive element

According to an aspect of the invention the at least one conductive bridge is positioned laterally with respect to the casing of the distal chamber and preferably positioned laterally such that the perpendicular distance between the non-insulated portion of the conductive element and the conductive bridge(s) during axial movement of the conductive element does not change more than 100%, not more than 50%, not more than 20%, suitably not more than 15%, preferably not more than 10%.

The non-insulated portion of the conductive element is disposed in a casing comprising an electrically insulating non-degradable material forming a distal chamber, the casing comprising a first structural component. The first structural component and optional second structural component enable the casing to be axially displaced with respect to the conductive element and to preserve a central disposition of the conductive element in the casing. For the first structural component and optional second structural component to slide in axial direction with respect to the insulated portion(s) of the conductive element there should be a void/lumen between the insulated portion of the conductive element and first and second structural components. Alternatively, the casing is movably disposed around first and optionally second structural components while said structural components are permanently attached to the insulated distal and proximal portions of the conductive element. The association of the first structural component with the proximal insulated portion of the conductive element should preferably be configured such that the electrical impedance between the non-insulated portion of the conductive element and the soft tissue (adjacent to the at least one conductive bridge) is lower than the electrical impedance inside the casing between the non-insulated portion of the conductive element and the tissue surrounding the proximal part of the proximal compartment or tissue proximally to the first structural component in case there is no proximal compartment.

For embodiments where the casing has an opening distally to a second structural component the impedance over said second structural component (between the non-insulated portion of the conductive element and the tissue surrounding the distal opening of the casing distally to the second structural component) is higher or significantly higher than the impedance between the non-insulated portion of the conductive element and the soft tissue adjacent any (lateral) conductive bridges.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by at least a first structural component which may form part of the casing, the first structural component extending in radial direction between the casing and the conductive element, the first structural component partitioning the casing in a distal chamber and a proximal compartment, the casing and the first structural component forming a distal chamber around the non-insulated portion of the conductive element, the casing and the first structural component essentially partitioning the distal chamber from the adjacent soft tissue, the casing being movably associated with the conductive element, wherein the casing comprises at least one electrically conductive bridge, the bridge configured to electrically connecting the distal chamber with adjacent soft tissue.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulated portion, at least part of the conductive element being disposed in a casing (envelope, sleeve, sheath) of an electrically insulating non-degradable material and the non-insulated portion of the conductive element being disposed in the casing, the conductive element being essentially centrally disposed in the casing by a first structural component which may form part of the casing, the first structural component extending in radial direction between the casing and the conductive element, the first structural component partitioning the casing in a distal chamber and a proximal compartment, the casing and the first structural component forming the distal chamber around the non-insulated portion of the conductive element, the casing and the first structural component essentially partitioning the distal chamber from the adjacent soft tissue, wherein either the first structural component is detached from the conductive element, or the casing is detached from the first structural component and wherein the casing comprises at least one electrically conductive bridge, the bridge configured to electrically connecting the chamber with adjacent soft tissue.

Some embodiments relate to a microelectrode configured to be at least partially embedded into or at least partially placed adjacent to soft tissue, in particular nervous, endocrine and muscle tissue, comprising an elongated electrically conductive element, the electrically conductive element comprising a proximal electrically insulated portion and a non-insulated portion distally to the proximal insulted portion, at least part of the conductive element being disposed in a casing (envelope) of electrically insulating non-degradable material, the casing comprising a first structural component which first structural component may form part of the casing, said first structural component partitioning the casing in a distal chamber and a proximal compartment, the non-insulated portion of the conductive element being encapsulated by the casing and the first structural component forming the distal chamber, the casing and the first structural component essentially partitioning the non-insulated portion of the conductive element from the adjacent soft tissue, the first structural component being detached from the conductive element, wherein the distal chamber comprises electrically conductive bridges electrically coupling the distal chamber with the adjacent soft tissue.

Some embodiments relate to a microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope) of electrically insulating non-degradable material the casing comprising a first structural component, which first structural component may form part of the casing, the first structural component partitioning the casing in a distal chamber and a proximal compartment, wherein the non-insulated portion of the conductive element is encapsulated (surrounded) by the casing forming the distal chamber, the first structural component being detached from the conductive element, wherein the casing of the distal chamber comprises at least one conductive bridge electrically coupling the distal chamber with the adjacent soft tissue.

Some embodiments relate to a microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope) of electrically insulating non-degradable material, the first structural component partitioning the casing in a distal chamber and a proximal compartment, wherein the non-insulated portion of the element is encapsulated (surrounded) by the casing forming the distal chamber, in which the conductive element can slide in an axial direction, the casing of the distal chamber comprising electrically conductive bridges electrically coupling the distal chamber with the adjacent soft tissue, and wherein the casing comprises a first structural component in which the electrically insulated portion of the conductive element can slide in an axial direction.

A restriction of charged particles through the lumen between the insulated portion of the conductive element and the first structural component is desirable in the event that for example the distal chamber and proximal compartment of the casing are disposed in different tissues comprising aqueous body fluid differing in composition, and that an exchange of aqueous body fluid between the tissues is to be minimized. This is, for instance, of importance when avoiding communication of cerebrospinal fluid with nervous tissue in the neighborhood of the distal element portion lacking insulation.

Since the casing is allowed to follow (adjust to) any movement of the surrounding soft tissue the conductive bridges of the casing of the distal chamber will essentially over time always be located at nearly the same spatial region in the soft tissue. Hence, the microelectrode of the invention will over time always monitor or stimulate essentially the very same region of the soft tissue. This characteristic is generally of importance for any soft tissue and of particular relevance for nervous tissue such a nervous tissue associated to the brain, dorsal root ganglia, spinal cord and peripheral nerves. The design of the microelectrode significantly improves over prior designs specifically in a dimension that nearly the same spatial region of the soft tissue is monitored and/or stimulated over time and even if soft tissue is displaced.

Electrically excitable cells, such as neurons, are found in any tissue susceptible to electric stimulation including nervous tissue, endocrine tissue, muscle tissue and connective tissue.

The casing comprises at least a first structural component which enables the casing, i.e. the casing defining the distal chamber, to slide axially with respect to the conductive element and specifically with respect to the insulated portion of the conductive element. This first structural component may optionally be an integral part of the casing but can also be provided by an element distinct from the casing. If the microelectrode only comprises a distal chamber the first structural component of the casing suitably constitutes a proximal portion of the casing of the distal chamber narrowing down to a configuration providing a slidable connection with the proximal electrically insulated portion of the conductive element while simultaneously minimizing the exchange of charged particles through any void between the proximal electrically insulated portion of the conductive element and the proximal portion of the casing of the distal chamber

In one embodiment, the casing comprises a first structural component partitioning the casing (envelope) in a distal chamber and a proximal compartment. The distal chamber is preferably essentially electrically insulated from adjacent soft tissue except for the electrical coupling comprised in the casing

By encapsulation of the distal chamber should be understood that the distal non-insulated portion of the electrically conductive element is essentially electrically isolated from the surrounding tissue by the casing except for the conductive bridge in the casing of the distal chamber. Some leak current will often be present over the lumen/void/annular channel between the insulated portion of the conductive element and the first structural component.

Depending on the production method, the first and second structural components may be an integral part of the casing, alternatively, the first and second structural components are element distinct from the casing optionally of a material different from the material of the casing (FIG. 23).

Irrespective if the casing forms only a distal chamber, or, a distal chamber and proximal compartment it is important that the casing can move with respect to the conductive element, specifically in axial direction. In an aspect of the invention, the casing encapsulates the distal non-insulated portion of the element. As the casing needs to be able to move axially with respect to the conductive element the casing should be slidably connected to or engaged with the proximal electrically insulated portion of the conductive element. For certain embodiments, the part of the casing slidably mounted/engaged/disposed around the proximal electrically insulated portion of the conductive element is referred to as the first structural component.

If first and optional second structural components form part of the casing there must be a void/lumen/gap between the structural components and the insulated portion of the conductive element. If first and optional second structural components are separate from the casing there is either a void/lumen/gap between the casing and the structural components, alternatively, if first and optional second structural components are separate but attached to the casing a void/lumen/gap between the structural components and the insulated portion(s) of the conductive element.

By ‘slidably mounted, engaged or disposed around’ is understood a mounting or engagement enabling axial movement while at least reducing the migration of charged particles (such as ions) over the first and optional second structural components, such as between the distal chamber and proximal compartment or proximal tissue to the first structural components (if the microelectrode lacks a proximal compartment) and optionally between the distal chamber and soft tissue distally to the distal end of the casing if the casing has a distal opening, said distal opening preferably positioned distally to a second structural component. Put differently, the slidable association of the casing to a proximal electrically insulated portion of the element and optionally slidably association of the casing with a distal insulated portion of the conductive element should provide a higher impedance between the distal chamber and proximal compartment (or surrounding soft tissue in case the casing only encapsulates the distal non-insulated portion of the element), or a higher impedance between the soft tissue adjacent the distal opening of the casing and the distal chamber, over the distance of the attachment (over first and second structural components) while simultaneously enabling an axial movement, than the impedance between the non-insulated portion of the conductive element and the soft tissue adjacent to the conductive bridges.

According to an aspect, the void/lumen/annular channel between the first and optional second structural components and the proximal electrically insulated portion of the conductive element and optional distal insulated portion of the conductive element, may comprise a composition which is essentially stable over time in tissue fluids and facilitates axial movement of the casing while minimizing migration of charged particles (and thus providing a high impedance over the first and optional structural component). According to an aspect, the composition which is essentially stable over time in tissue fluids and facilitates axial movement of the casing while minimizing migration of charged particles may be a composition facilitating the movement of the first and optional second structural component with respect to the outermost layer of the insulated conductive element or between the outermost layer of first and second structural components and the inside of the casing, particularly a composition comprising any one of lipids, silicones (such as silicone oil or silicone grease) and combinations thereof.

Some embodiments, such as microelectrodes, microelectrode probes and arrays, comprise a biocompatible material providing rigidity to the probe when dry for insertion into soft tissue and dissolvable or degradable in aqueous body fluids. The term rigidity when dry should be interpreted as a dryness causing the material to crack under load (radial or axial load) instead of bending.

Useful biocompatible materials providing sufficient rigidity to the probe when dry for insertion into soft tissue and dissolvable or degradable in aqueous body fluids. The biocompatible materials, also referred to as matrices, are suitably chosen from protein-based (proteinaceous) materials, carbohydrate-based materials, and polyethylene glycols of various molecular weights. A suitable protein-based matrix material is gelatin typically derived from collagen. A suitable carbohydrate-based matrix material is glucose. The biocompatible matrix material may be selected from gelatin, glucose and polyethylene glycol.

According to an embodiment, microelectrodes or microelectrode probes may be attached to a non-degradable matrix thereby forming a non-degradable array.

When the microelectrodes or microelectrode probes are attached to a non-degradable matrix it is important that at least some of the performance providing elements of the microelectrode (e.g. openings, electrically conductive bridges) are not impaired by the non-degradable matrix.

The extension of the non-degradable matrix with respect to the microelectrodes has a configuration that most of the performance providing elements of the microelectrode are not impaired. Preferably, the non-degradable matrix is arranged with respect to the microelectrodes or microelectrode probes such that most of the openings and/or electrically conductive bridges, preferably more than 50%, more than 60%, more than 70%, suitably essentially all of the openings and/or electrically conductive bridges, are not embedded by the non-degradable matrix. According to a version, the micro- or nanofibers are non-degradable and attached to the microelectrodes by a non-degradable adhesive. Non-degradable material/matrix should be understood as a material not essentially affected in presence of body fluids. The non-degradable matrix is preferably flexible. The non-degradable matrix preferably comprises polymers being flexible at body temperatures. Suitable matrix materials can be selected from silicones and any of the polymers used for the casing but not limited to those materials. This non-degradable matrix should not be confused with the term ‘matrix’ or ‘array matrix’. The terms ‘matrix’ or ‘array matric’ relate to degradable and/or disintegrating matrices in tissue fluids. The matrices found in the distal chamber and the proximal compartment and matrices defined as array matrices are characterized as biocompatible materials providing sufficient rigidity to the probe when dry for insertion into soft tissue and dissolvable or degradable in aqueous body fluids. The non-degradable matrix does not dissolve and degrade upon insertion into soft tissue. Furthermore, the non-degradable matrix is suitably flexible or becomes flexible upon insertion into soft tissue.

According to a specific embodiment, the soft tissue may assume the function of a non-degradable matrix. A plurality of microelectrodes or microelectrode probes may be positioned into soft tissue according to a predetermined spatial pattern. The soft tissue per se secures the positioning of the microelectrodes over time.

According to all embodiments the insulation material of the insulated portion of the conductive element is non-degradable in body fluids. The insulation material may be chosen from any materials of the casing but is not limited to those materials.

Should the first and optional second structural component be distinct to the casing, it is important that the attachment of the first and optional structural component to the casing prohibits migration of charged particles. The material of the first and optional second structural component must also be essentially electrically non-conductive.

According to an aspect, the first and optional structural component has an extension in axial direction of at least from about 5 μm up to about 10 mm, preferably from about 5 μm up to about 3 mm.

According to an aspect, at least part of the electrically insulated portion is localized within the distal chamber.

According to a further aspect, a lumen/void (enabling axial movements) is provided between the first and optional second structural component and the electrically insulated portion of the conductive element.

The lumen/void may also be contemplated as an annular channel formed between the first and optional second structural component and the electrically insulated portion of the conductive element.

It is preferred that the lumen/void/annular channel between the first and optional second structural component and the electrically insulated element restricts radial movements of the conductive element with respect to the distal casing and that the impedance over this lumen/void is higher than the impedance over the opening(s) in the distal casing.

According to a further aspect, the proximal portion and optionally distal portion of the distal chamber narrows down, exhibiting an annular structure forming the first and optional second structural component, in which the electrically insulated portion of the conductive element can slide in an axial direction.

It is important that the entire casing can move, typically in axial direction, with respect to the conductive element, thereby following the movement of the adjacent soft tissue.

According to an aspect, the innermost material(s) of the casing and/or the first and optional second structural components and/or the outermost material of the proximal electrically insulated portion of the element is/are (each) selected to reduce friction.

The first and optional structural component may be provided by any shape of the casing or non-casing component enabling the casing to move in axial direction with respect to the insulated conductive element and suitably providing a higher impedance over the first and optional second structural component in relation to the impedance between the non-insulated portion of the conductive element and the conductive bridges in the distal casing which renders useful recordings and stimulation of electrically excitable cells (neurons) adjacent to the at least one opening and/or electrically conductive bridges in the casing of the distal chamber.

According to an aspect, the electrical impedance between the non-insulated portion of the conductive element and the soft tissue (adjacent to the at least a conductive bridges) is lower than the electrical impedance within the casing between the non-insulated portion of the conductive element and the tissue surrounding the proximal part of the proximal compartment or tissue proximally to the first structural component (in case there is no proximal compartment) and optionally tissue adjacent to the opening in the casing distally to an optional second structural component.

According to a further aspect, the electrical impedance between the non-insulated portion of the conductive element and the soft tissue (adjacent to the at least one conductive bridges), is at least 5 times lower, preferably at least 25 times lower, preferably at least 100 times lower, than the electrical impedance between the non-insulated portion of the conductive element and the tissue surrounding the proximal part of the proximal compartment or tissue proximally to the first structural component and optionally tissue adjacent to the opening in the casing distally to an optional second structural component.

Preferably, the electrical impedance between the non-insulated portion of the conductive element and the soft tissue (adjacent to the at least one conductive bridge) is lower, preferably at least 5 times lower, preferably at least 25 times lower, preferably at least 100 times lower, than the electrical impedance over the first and optional second structural components.

It is preferred that the axial movement of the non-insulated portion of the conductive element does not significantly influence the radial positioning within the distal casing. Preferably, the perpendicular distance (FIG. 24, 104) between the non-insulated portion of the conductive element and the at least one conductive bridge of the casing of the distal chamber remains essentially the same during axial movements of the casing relative to the conductive element, optionally less than 20%.

A variation of the distance of the non-insulated portion of the conductive element to the conductive bridge will inevitably lead to a variation in the distance to the monitored tissue (adjacent to the respective conductive bridge) which will have an impact on the fingerprint of the recorded signals. A variation of the distance may induce amplitude variance of recorded signals interfering with the ability to distinguish signals from unique cells.

According to an aspect the distal chamber comprises a second structural component configured to reducing radial movement of the non-insulated portion of the conductive element relative to the distal casing, while also being configured to enable an axial movement of the non-isolated conductive element with respect to the second structural component. This second structural component may form part of the casing, thus being an integral part of the casing. However, the second structural component may also be distinct from the casing. For example, the second structural component may be of e.g. Teflon, attached to the casing and comprising a central channel enabling the non-insulated portion of the conductive element to move in an axial direction.

According to an aspect, the material of the second structural component is distinct from the material of the casing being at least partly attached to the casing and configured to be slidably engaged with the non-isolated conductive element.

The proximal insulated portion of the conductive element may comprise a segment which facilitates flexing in axial and radial direction.

According to an aspect, the distal portion of the casing of the distal chamber has a three-dimensional shape narrowing in distal direction. Such a three-dimensional shape may be spherical, paraboloid (elliptically paraboloid), or conical.

It is preferred that the casing accommodates for movements of the soft tissue while the conductive element can move with respect to the casing.

According to an aspect, the casing comprises means for increasing friction (or put differently for anchoring the casing in the soft tissue) between the casing and the adjacent soft tissue. Preferably, the means for increasing friction is selected from micro- or nano-fibers attached to the outermost surface of the casing.

According to an embodiment, the micro- or nano-fibers may be non-degradable to body fluids. Thus, the material of the micro- or nano-fibers is not degradable when in contact with body tissue.

Thus, according to an aspect, the friction between the casing and the adjacent soft tissue is higher than the friction between the innermost material of the casing and/or the first and optional second structural component and/or the outermost material of the proximal electrically insulated portions of the element.

A further aspect is that the outermost material and/or outermost surface structure of the casing is selected to increase friction against the soft tissue.

According to yet a further aspect, the casing comprises two layers of materials an inner layer and outer layer, wherein the material of the inner layer is different from the material of the outer layer or wherein the surface structure of the inner layer is different from surface structure of the outer layer. The casing may be configured such that the volume of the casing varies. The movement of the casing with respect to the conductive element (and the insulated section of the conductive element) will temporarily have an effect on the pressure inside the casing. The casing is preferably configured to counteract pressure variance caused by the mutual movement of the conductive element and casing. If the mutual movement temporarily increases the pressure inside the casing the volume of the casing increases. If the mutual movement temporarily decreases the pressure inside the casing the volume of the casing decreases. The casing may comprise a flexible and elastic polymeric material, such as silicon.

A further measure to counteract pressure variability caused by the movement of the conductive element with respect to the casing is the provision of an opening in the casing distally to the second structural component, and the presence of first and a second structural components, the structural components movably disposed around distal and proximal insulated portions of the conductive element. A variation of pressure in the distal chamber (restricted by first, second structural components and casing) is counteracted by the presence of the first and second structural components in combination with the insulating portions. Additionally, and importantly, an opening in the casing distally to the second structural component significantly reduces or essentially eliminates pressure variability which would otherwise occur in a cavity distally to the second structural component fully enclosed by the casing and induced by the axial movement of the distal end portion of the distal insulated portion.

The microelectrode may comprise an engagement element configured to reversibly engage with an elongated rigid pin, such as a needle, the rigid pin being configured to insert the microelectrode into the soft tissue or placing the microelectrode adjacent to soft tissue. The engagement element is suitably positioned at the distal tip of the microelectrode but can also be positioned along the distal casing. Thus, the engagement element may be positioned at a distal portion of the casing, such as the distal portion of the distal casing, including the distal tip of the distal casing. If the microelectrode comprises an engagement element the microelectrode may be inserted into soft tissue or positioned adjacent to soft tissue by way of a rigid pin reversibly engaging with the engagement element, the rigid pin (such as a needle) forming part of an apparatus for inserting microelectrodes into soft tissue as disclosed by e.g. US 2020/0086111 A1. If a microelectrode is inserted using a rigid pin (reversibly engaging with an engagement element of the microelectrode) there is less need for that the microelectrode per se exhibit an enhanced intrinsic rigidity during the insertion into the soft tissue. Thus, a microelectrode comprising an engagement element may at least partly dispense with any material providing the microelectrode with rigidity, such as a biocompatible material providing sufficient rigidity to the probe when dry for insertion into soft tissue and dissolvable or degradable in aqueous body fluids.

The engagement element may constitute a loop or comprise a net. According to an aspect the engagement element may also constitute non-degradable or degradable micro- or nanofibers, the micro- or nano-fibers being adhesively attached to the microelectrode, typically attached to the casing, specifically to the distal section of the casing, such as the distal casing. The microfibers may be any of the micro- or nano-fibers disclosed herein.

According to an embodiment the microelectrode comprises a biocompatible material providing sufficient rigidity to the probe/microelectrode when dry for insertion into soft tissue and dissolvable or degradable in aqueous body fluids. The material imparting structural rigidity to the microelectrode is typically found in the distal chamber and optionally also present in the proximal compartment or around at least part of the insulated portion of the conductive element.

The microelectrode may also be disposed in a material providing sufficient rigidity to the probe when dry for insertion into soft tissue and dissolvable or degradable in aqueous body fluids. One may contemplate a microelectrode comprising a casing and a distal chamber and optionally a proximal compartment, where the distal chamber and the optional proximal compartment does not comprise a biocompatible material providing sufficient rigidity, yet, the microelectrode being disposed in a biocompatible material providing sufficient rigidity.

A microelectrode comprising biocompatible material increasing rigidity is herein also referred to as a microelectrode probe.

Also disclosed is a microelectrode (or proximal bridging arrangement) comprising a conductive element comprising distal and proximal non-insulated sections and further an insulated section between said distal and proximal non-insulated section, where the insulated section of the conductive element is slidably associated with a casing encapsulating at least the proximal non-insulated section of the conductive element, the casing further insulating the proximal non-insulated section of the conductive element from direct contact with adjacent soft tissue once the microelectrode is inserted into soft tissue forming a proximal lumen, the casing exposing conductive material capable of electrically coupling the proximal non-insulated section of the conductive element with a second conductive element (conductive lead) electrically coupled to the conductive material of the casing. The casing encapsulating the proximal non-insulated section of the conductive element may constitute a conductive material such as a metal or conductive polymer. The casing engages with the insulated section of the conductive element via a fifth structural component. This fifth structural component may be integrated in the casing, alternatively, the fifth structural component may be distinct to the casing. If the fifth structural component is distinct to the casing the casing is attached to a distinct fifth structural component. The casing may be covered by a layer of electrically insulating material, which is preferably flexible and may be selected from parylene C or silicone. The casing or conductive material exposed to fluid inside the proximal lumen is electrically coupled to a second conductive element (conductive lead) connecting the microelectrode to a suitable electronic device for stimulation and/or recording purposes. The insulated section of the conductive element preferably comprises at least one structural element limiting the axial movement of the conductive element. Such structural element may increase the radial extension of the insulation at a location close to the proximal end of the insulation. Such a structural element prohibits the conductive element from separating from the casing. The microelectrode disclosed in this very paragraph (also referred to as proximal bridging arrangement) may form part of any of the microelectrodes defined as inter alia comprising an electrically conductive element comprising a proximal electrically insulated portion and distal non-insulated portion, the distal non-insulated portion of the conductive element and preferably at least part of the insulated conductive element being disposed in a casing (envelope) of electrically insulating non-degradable material, the non-insulated portion of the conductive element being encapsulated (surrounded) by the casing forming a distal chamber, in which the conductive element can slide in an axial direction.

Also, the proximal bridging arrangement can be associated with any microelectrode comprising a proximal non-insulated electrode (proximal non-insulated conductive electrode).

A further embodiment of the invention relates to arrays, of microelectrodes and/or microelectrode probes. In its widest definition an array is characterized by at least two microelectrodes/microelectrode probes, the array structure capable of being implanted into soft tissue or positioned adjacent to soft tissue, a number of microelectrodes/probes disposed in a set spatial conformation without essentially changing the disposition during insertion. An array is typically provided by embedding microelectrodes and/or microelectrode probes, in a matrix. The matrix may be a non-degradable matrix or a dissolvable and/or degradable matrix. An array may comprise both a non-degradable matrix dissolvable and/or degradable matrix. If an array comprises both a non-degradable matrix dissolvable and/or degradable matrix, the non-degradable matrix is suitably in contact with the microelectrodes/microelectrode probes further forming a continuous matrix connecting all microelectrodes or microelectrode probes. An array may constitute a plurality of individual microelectrodes and/or microelectrode probes arranged in various three-dimensional shapes.

According to an embodiment, a plurality of microelectrodes and/or microelectrode probes are attached to a non-degradable matrix and/or adhesively attached to micro- or nanofibers. Preferably, the non-degradable matrix is arranged with respect to the microelectrodes or microelectrode probes such that most of the openings and/or electrically conductive bridges, preferably more than 50%, more than 60%, more than 70%, suitably essentially all of the openings and/or electrically conductive bridges, are not embedded by the non-degradable matrix.

According to a version, the micro- or nanofibers are non-degradable and attached to the microelectrodes by a non-degradable adhesive. Non-degradable material/matrix should be understood as a material not essentially affected in presence of body fluids.

The non-degradable matrix is preferably flexible. The non-degradable matrix preferably comprises polymers being flexible at body temperatures. Suitable non-degradable matrix materials can be selected from silicones and any of the polymers used for the casing but not limited to those materials.

According to a specific embodiment, the soft tissue and in certain circumstances also hard tissue (like osseous skull tissue) may serve a similar purpose as a non-degradable matrix connecting a plurality of microelectrodes or microelectrode probes. A plurality of microelectrodes or microelectrode probes are positioned into the soft tissue according to a predetermined spatial pattern. The soft tissue per se secures the positioning of the microelectrodes over time. The individual microelectrodes/probes may be arranged in any conceivable spatial configuration in an array. Configurations may embrace axial sections, each section comprising a plurality of individual microelectrode having same of different spatial configurations.

According to an embodiment of an array, the microelectrodes are disposed substantially in parallel.

According to an embodiment of an array a plurality of microelectrodes and/or microelectrode probes are disposed essentially in parallel and essentially in one sheet. Preferably, the microelectrodes and/or microelectrode probes disposed essentially in parallel and essentially in one sheet are attached to a non-degradable matrix and/or adhesively attached to non-degradable micro- or nanofibers thereby forming an array. The adhesive is suitably non-degradable. The sheet formed array may be formed into any three-dimensional configuration adapted to the three-dimensional configuration of the target tissue. For example, the array sheet may be configured as a curved structure for the positioning adjacent the spinal cord between the spinal cord and the vertebra column or configured as a cuff for the positioning around a peripheral nerve.

According to an embodiment the microelectrodes and/or microelectrode probes of an array, such as a sheet-formed array or an array of a sheet-like configuration, are arranged such that most of the at least one opening and/or the electrically conductive bridges of the distal chamber face(s) in essentially the same direction. If the array has a sheet-like configuration most (at least about 50%, 70%, at least about 90%, preferably essentially all) of the openings and/or the electrically conductive bridges of the distal chambers of the microelectrodes and/or microelectrode probes faces the same side of the sheet-like configuration which is a disposition of the opening and/or the electrically conductive bridges that they face the soft tissue to be monitored and/or stimulated.

According to yet a further embodiment the microelectrodes are disposed to each other substantially in parallel in an array configuration comprising non-degradable micro- and/or nanofibers and/or a non-degradable matrix. The non-degradable micro- and/or nanofibers and/or non-degradable matrix secure the position of the microelectrodes with respect to each other over time. The net-like structure also allows for accommodation to volume changes in e.g. peripheral nerves. A plurality of microelectrodes may be disposed in an array to form a three-dimensional configuration, such as a cuff, configured to adjust to a peripheral nerve. Put differently, an array may comprise a plurality of microelectrodes disposed essentially parallel to each other further comprising non-degradable micro- and/or nanofibers and/or non-degradable matrix securing the position of the microelectrodes with respect to each other over time, the array having a configuration which allows the array to be positioned between the vertebra and the spinal cord (e.g. between vertebra body and the spinal cord or between the dura mater and the spinal cord). The array may have a curved configuration which is capable of partly enclosing the spinal cord. The array is preferably disposed between the vertebra and the dura mater of the spinal cord. However, the array may also be positioned in direct apposition to the spinal cord or between the arachnoid and dura mater or even between the pia mater and the arachnoid. The array may also be positioned in close apposition to dorsal and ventral roots, dorsal root ganglia or nerves. The array may also comprise structural elements which attach to the surrounding tissue. Such structural elements may be comprised in the micro- and/or nanofibers and/or be comprised in the casing of the microelectrodes. The microelectrodes may be disposed essentially in parallel in the distal region of the array comprising the casings of the microelectrodes but are preferably bundled together at some distance proximally to the casing and proximal to the non-degradable matrix in such way that the insulated conductive elements can move in axial direction.

If an array comprises a plurality of microelectrodes, such as three or more, a variant is that the axis of one microelectrode essentially coincides with the main axis of the array with remaining microelectrodes positioned radially around the axis of the array. Furthermore, the distal ends of the microelectrode may be disposed essentially in a plane perpendicular to the axes of the microelectrodes.

The arrays may have a configuration that associates microelectrodes/probes with each other. One type of association limits movements of microelectrodes with respect to each other. The adhesive attachment of microelectrodes with micro- or nano fibers of the first arrays is a means to limit movements of microelectrodes to each other. An array configured to associate microelectrodes may also be referred to as a bundle of microelectrodes. E.g. the microelectrodes, such as the casings of the microelectrodes, may be adhesively attached to each other either intermittently or permanently (e.g. by non-degradable materials such as non-degradable glues). Alternatively, the microelectrodes of an array are arranged to move independently after being inserted into soft tissue. In this variant the microelectrodes are spatially positioned only by a dissolvable or degradable array matrix.

According to an embodiment the array comprises an array cover. The array matrix may be configured to extend to the distal face of the array cover.

According to a further embodiment the array matrix may be in part be covered by an array casing of any of the electrically insulating material presented herein.

According to yet a further embodiment, the array may comprise a further outer array matrix.

A further aspect of the invention relates to a microelectrode probe. The microelectrode probe comprises features enabling the successful implantation of the probe by the insertion into soft tissue. Thus, the microelectrode probe comprises, with respect to the microelectrode, components providing the probe with sufficient rigidity to be inserted into soft tissue. Alternatively, the microelectrode may be transformed into a probe by altering the rigidity of materials of the microelectrode, typically the casing, enabling the insertion of the microelectrode into soft tissue for example by altering the temperature of the materials transiently.

According to an embodiment the distal section of the distal chamber narrows in distal direction. Preferably, the distal section of the distal chamber is of the same material as the casing. The distal section of the distal chamber provides for an axial movement of the non-insulated conductive element in the distal direction past the locations(s) of the openings and/or electrically conductive bridges.

According to an embodiment, the casing comprises a first structural component partitioning the casing (envelope) in a distal and proximal compartment, the distal chamber encapsulating the distal non-insulated portion of the element except for an opening and/or electrically conductive bridges in the casing. The casing serves several purposes. The casing is configured to enable it to move in axial direction with respect to the conductive element. Furthermore, the casing is configured to partition/divide the casing into a proximal and distal chamber by way of a first structural component. The first structural component may constitute an integral part of the casing. Alternatively, the first structural component may constitute a separate entity with respect to the casing. In the former, the first structural component shares the same material as the casing. In the latter, the first structural component may be of a different material than the casing. In certain embodiments, it is preferred that the microelectrode is configured such that the physical contact of the conductive element with the casing is minimized specifically with the distal non-insulated portion of the element. Apparent lateral movements of the conductive element with respect to the casing tend to be a function of the distance from the tubular structure of the first structural component. Hence, the distal tip of the non-insulated element tends to have a more pronounced lateral movement with respect to the casing than the part of the element closer to the first structural component.

In principle, the casing can have any form as long as the conductive element can be disposed within the casing. It may be favorable that the casing is rotationally symmetric in an effort to avoid the element to contact the casing. The casing can be rectangular or rhombic. According to one embodiment, the casing is rotationally symmetric typically with respect to a central axis normally coinciding with the main axis of the conductive element. The three-dimensional form of the casing may have an impact on the rigidity of the casing. Hence, the rigidity of the casing can be modulated not only by way of the choice of casing material but also the choice of three-dimensional form of the casing. One preferred three-dimensional form of the casing is the cylindric form optionally comprising configurations facilitating volume change of the casing such as an accordion-like configuration. Preferably, the conductive element is disposed in a casing of essentially cylindric form where the element essentially coincides with the main axis of the cylindrically formed casing.

As alluded to above, the casing is the prime facilitator for letting surrounding soft tissue not significantly interfere with the conductive element in general and specifically the distal non-insulated portion of the element present in the distal chamber. The casing may be attached to a first and optional second structural components which may have the form of a tubular structure enabling charged particles to pass between the distal chamber and adjacent tissue through the lumen/void between the proximal insulated portion and optional distal insulated portion of the conductive element and the first and optional second structural components. Should the first and optional second structural components be an entity distinct from the casing, the first and optional second structural components suitably abut and/or adhere to the casing. The first and optional second structural components suitably comprise an arrangement such as an elongated tube, preferably with an annular cross-section, preferably configured to provide a lumen/void between the insulated portions of the conductive element and the elongated tube (tubular structure). The volume of the lumen/void enables the movement of the first and optional second structural components with respect to the conductive element (insulated portions of the conductive element), preferably an axial movement of the first and optional second structural component.

According to an embodiment, the void/lumen (defined by the space between the proximal and optional distal electrically insulated portion of the conductive element and the first and optional second structural component) has an extension in axial direction satisfying as least one of the following criteria: a) allowing the first and optional second structural components (e.g. tubular structure) to move with respect to the conductive element, b) allowing the first and optional second structural component to move with respect to the conductive element while simultaneously centralizing the casing with respect to the axis of the conductive element (first and optional second structural components enabling an essentially centrally disposition of the conductive element within the casing), c) providing a difference in terms of the electric impedance emergent between the proximal compartment and distal chamber and distal chamber and tissue adjacent to an opening in the casing laterally to a second structural component (over the first and optional second structural components) on the one hand and the electrical impedance emergent between the distal non-insulated portion of the conductive element and the (surrounding) soft tissue one the other hand.

If the microelectrode probe comprising proximal and distal chambers is not embedded in a dissolvable and/or degradable embedding matrix it is preferred to apply a further degradable and/or dissolvable matrix in the space between the proximal compartment and distal chamber referred to as an intermediate matrix. The radial extension of the intermediate matrix suitably follows the radial extensions of the proximal compartment and distal chamber. The degradable and/or dissolvable embedding and intermediate matrix provide increased rigidity to the microelectrode during insertion into soft tissue.

The electric impedance emerging between the proximal compartment and distal chamber and distal chamber and between tissue adjacent to an opening in the casing laterally to a second structural component and the distal chamber (over the first and optional second structural components) is to an extent a function of the extension of the void/lumen in axial and radial direction and the volume of the void/lumen between the first and optional second structural components (tubular structure) and the conductive element or between the first and optional second structural components and the casing. At a given extension of the first and optional second structural components a reduction of the volume of the void/lumen will increase the electric impedance between the proximal compartment and distal chamber, and between the distal chamber and tissue adjacent to an opening in the casing laterally to a second structural component.

The greater the axial extension of the void/lumen the higher the impedance at a given area of the void/lumen in a plane perpendicular to the axis of the element (and implicitly the microelectrode). An increase in axial extension of the void/lumen also tends to increase the friction between the conductive element and the first and optional second structural components. The axial extension of the void/lumen must satisfy the criteria of providing a sufficiently high electrical impedance while enabling the first and optional second structural components to slide with respect to the conductive element.

According to an embodiment, the friction between the casing and the surrounding soft tissue is higher, preferably significantly higher, than the friction between the conductive element and the casing (including first and optional second structural components). The difference in friction is as least such that useful patterns of data can be extracted from the microelectrode. Specifically, the difference in friction is at least such that useful patterns of data can be extracted from the same region of the soft tissue over time.

For certain embodiments, the axial extension of the distal casing, defining a distal chamber in particular the void distal to the non-insulated conductive element and the axial extension of the first structural component is partly correlated to the normally occurring displacements of the soft tissue abutting the conductive bridges in the distal casing in relation to a proximal connection, typically localized in the skull or vertebra. Thus, the extension of the void/lumen of the casing (or the first structural component) is dependent on the spatial movements of the respective tissue. The extension of the void/lumen in axial direction (i.e. the extension of the distal chamber in axial direction) may broadly range from at least about 300 μm up to about 20 mm but can be longer.

The materials of the casing and the outermost material surrounding the conductive element or outermost material if first and second structural components (if structural components are attached to the insulated portions of the conductive electrode) may be selected with the aim of facilitating the movement of the first and optional second structural component with respect to the insulated portion of the conductive element in axial direction.

The outermost material surrounding the conductive element at the location of the first and optional second structural component (or outermost material surrounding the first and second structural components) may constitute the electrical insulation per se. Furthermore, the void/lumen between the inner surface of the first and optional second structural component and the outermost material surrounding the conductive element (or the void/lumen between casing the first and second structural components) may comprise a composition (medium) facilitating the axial movement of the first and optional second structural component with respect to the conductive element (or casing with respect to the first and second structural components). Such a composition may be selected from lipids, silicones and compositions comprising hyaluronic acid and a polymer of disaccharides or a composition mimicking the characteristics of synovial fluid.

A further embodiment of the microelectrode comprises a second structural component configured to minimize lateral (radial) movements of the distal non-insulated portion of the element. The second structural component should also allow the element to move in axial direction. Several second structural components may be positioned within the distal chamber for positioning the element centrally. The second structural component may be integrated with the casing and adhere to the inner surface of the casing or optionally being made of the same material as the casing and integral with the casing. Alternatively, the second structural component may be distinct from the casing preferably made of materials other than casing materials.

Lateral movements of the distal non-insulated portion of the element with respect to the casing and specifically with respect to the conductive bridges may alter the shortest distance between the distal non-insulated portion of the element and the soft tissue and, hence, have an implication for the impedance between the distal non-insulated portion of the element and the soft tissue which in turn may affect the measurement/stimulation.

The dimensions of the microelectrode are such that materials may be used for the casing which are stiff at macroscopic dimensions but become sufficiently flexible at the dimensions of the microelectrode. Hence, various crystalline materials may be contemplated as casing materials, such as crystalline materials comprising silicon dioxide such as any material referred to as glass. According to a preferred embodiment, the electrically insulating material is an electrically insulating non-degradable flexible polymeric material. Suitable electrically insulating non-degradable flexible polymeric materials are polymeric materials which can be disposed by dip coating, spray coating, vapor deposition or casting or any combination thereof. Suitable electrically insulating flexible non-degradable polymeric materials include polytetrafluoreten (Teflon), Parylene C, polyurethanes, polyethylenes and polymers comprising a backbone of recurring aromatic moieties such as aromatic moieties comprising an aromatic six-membered ring structure exemplified by para benzenediyl moieties. Preferred polymeric materials are polymers obtained by the polymerization of para-xylene. Hydrogen atoms of the polymers comprising a backbone of recurring aromatic moieties may be substituted by various functional groups. Parylenes are a preferred class of electrically insulating flexible polymeric materials sharing the characteristics of polymers comprising a backbone of recurring aromatic moieties such as aromatic moieties comprising an aromatic six-membered ring structure exemplified by bara benzenediyl moieties. The polymeric materials may be chosen from for example Parylene C and Parylene M.

The thickness/diameter of the casing may range from about 0.1 μm, from about 4 μm, from about 10 to 15 μm up to about 300 μm and for some embodiments up to about 1 to 2 mm.

All materials of the microelectrode that are in contact with tissue, such as electrically insulating materials and lateral member materials, must be biocompatible.

According to a further embodiment the proximal electrically insulated portion of the conductive element is configured to accommodate for spatial movements of the soft tissue. The proximal electrically insulated portion of the conductive element may comprise at least one section facilitating flexing of the element particularly flexing in a direction partly coinciding with the main axis of the conductive element (microelectrode) and/or a section facilitating bending in radial direction. This flexing section of the element may be localized proximally to the proximal compartment between the proximal compartment and a holder. Alternatively, the flexing section may be localized within the proximal compartment, i.e. fully disposed in the casing of the proximal compartment. The section facilitating flexing enables the proximal electrically insulated portion of the conductive element to be elongated by at least about 10% (based on the length of the proximal insulated portion in equilibrium state), at least about 20%, at least about 50% and preferably at least about 100%. The section facilitating elongation (flexing) of the electrically insulated portion of the element can be chosen from any of the following forms: spiral form, zig-zag-form, meandering form, or any combination of the forms.

The material of the electrically conductive element can be any electrically conductive material fulfilling the characteristics of a microelectrode for implantation into soft tissue, specifically neural, endocrine or muscular tissue. A variety of metals are suitable, but also conductive non-metal materials. Suitable materials are metals or mixtures of metals in the tissue surrounding the microelectrode, including platinum, iridium, gold, wolfram, stainless steel, and alloys thereof. More preferred metals of the conductive element are selected from materials that do not easily oxidize such as platinum, iridium, gold, stainless steel and alloys thereof. Conductive non-metal materials include for example various conductive polymers such as PEDOT and carbon-containing materials such as graphene, graphite and carbon nanotubes.

The conductive element can be of a single metal or comprise two or more portions of different metals. Alternatively, the element can comprise two or more ultra-thin metallic wires. The thickness of the one or more wires is preferably from about 100 nm to 1 μm or 10 μm or even 100 μm. The two or more ultra-thin wires may be entangled such that the surface area is maximized.

The section of the electrically insulated portion of the conductive element extending proximally of the proximal compartment can be of a material or of materials different from that or those of the portion disposed in the proximal compartment and distal chamber. The non-insulated portion of the conductive element present within the distal chamber may exhibit sections of the surface with a higher surface area than the average surface area of the non-insulated portion of the element within the distal chamber. Suitably, the sections(s) exhibiting a higher surface area is (are) localized in the vicinity of the conductive bridges of the distal chamber. The non-insulated portion of the element present in the distal chamber may also comprise rugged sections or comprise protrusions near the conductive bridges. The rugged sections or protrusions are in the micro or nano scale.

As recited in the claims the distal non-insulated portion of the element is entirely localized within the distal chamber.

According so some embodiments, during operation of the microelectrode, the most distal section of the insulated proximal portion of the element should preferably always be comprised in the distal chamber. The casing should suitably, under normal conditions, be positioned in relation to the conductive element such that the casing fully embraces the non-insulated portion of the conductive element irrespective of axial movement of the casing. Also, according to some embodiments the conductive element should be originally positioned in the casing such that the distal tip of the non-insulating portion of the conductive element never reaches the casing of the distal chamber. Alternatively, the microelectrode may have a means which limits the axial movement of either the casing or the conductive element such that the distal tip of the non-insulated conductive element may never contact the casing or puncture the casing.

According to some embodiments the first and optionally second structural component may be positioned initially at a location with respect to the insulated proximal portion of the conductive element that the probability that the first and optional second structural components will to an extent leave the insulated portion of the element (entirely or partially slide over the non-insulated portion) is minimal or virtually non-existent.

Implanted microelectrodes may need to be removed from the surrounding tissue. In order to facilitate the removal the microelectrode may comprise a flexible filament securely attached to the microelectrode at a location facilitating the removal. The proximal portion of such flexible filament should be located such that the filament is easily retrievable without undue irritation of any tissue. Alternatively, the distal part of the non-insulated conductive element can be equipped with a rounded blob having a dimension such that it cannot be pulled through the first or optional second structural component. In this case the entire microelectrode can be extracted from the tissue by pulling out the conductive filament.

For certain embodiments of the microelectrode comprising a first and second structural components and first and second structural components form part of the casing or first and second structural components are separate from the casing and attached to the casing, the diameter of the distal insulated portion of the conductive element is larger than the inner diameter of the first structural components and preferably also larger than the diameter of the proximal insulated conductive element. When such a microelectrode is withdrawn from the soft tissue the distal insulated portion is prohibited from sliding though the first structural component. As the casing is attached to the first and second structural component the complete microelectrode is withdrawn from the soft tissue by pulling the conductive element or any lead attached to the conductive element. An alternative solution is that the distal insulated portion comprises a protrusion positioned distally to the second structural component prohibiting the distal insulated portion of the conductive element to completely slide through the second structural component when the conductive element pulled in proximal direction.

A further aspect of the invention relates to a microelectrode probe. As already alluded to above the microelectrode probe constitutes a version of the microelectrode which is designed to be inserted into soft tissue. Hence, the microelectrode probe comprises certain components providing the probe with sufficient rigidity to be successfully inserted into various soft tissues. Once inserted into soft tissue, certain components of the microelectrode probe dissolves and/or disintegrates upon contact with body fluids transforming the microelectrode gradually into the microelectrode, an in-situ microelectrode.

The microelectrode probe comprises matrices of biocompatible materials providing sufficient rigidity to the probe and also holding together the different components (casing and conductive elements) of the microelectrode when dry for insertion into soft tissue and dissolvable or degradable in aqueous body fluids. The matrices are suitably chosen from protein-based (proteinaceous) materials, carbohydrate-based materials, and polyethylene glycols of various molecular weights. A suitable protein-based matrix material is gelatin typically derived from collagen. A suitable carbohydrate-based matrix material is glucose. The biocompatible matrix material may be selected from gelatin, glucose and polyethylene glycol. The distal chamber which is encapsulated by the casing should preferably comprise a matrix material that does not significantly increase its volume when dissolving in aqueous fluids. The matrix of the distal chamber may have the characteristics that the increase in volume of the matrix when absorbing an aqueous fluid is in part counteracted by the dissolution/degradation of the matrix.

Matrix materials increasing their volume when absorbing an aqueous fluid may be used for embedding matrices or for cavities/compartments provided by casing materials sufficiently flexible for not undergoing structural damages during matrix volume expansion.

Any of the variants/embodiments of the microelectrode presented herein may be provided as a microelectrode probe.

One variant of the microelectrode comprises a casing encapsulating the distal non-insulated portion of the element forming the distal chamber but lacks a proximal compartment. The microelectrode probe of this ‘one-compartment’ variant of the microelectrode comprises a distal matrix. It is furthermore preferred to have a proximal matrix around part of the proximal insulated portion of the conductive element. Preferably, this proximal matrix has a spatial radial extension similar to the spatial radial extension of the distal chamber. The proximal matrix may enclose a rigid pin/bar used when inserting the microelectrode. It is preferred that the pin has the same main axis as the distal chamber.

Thus, in one embodiment of the invention the microelectrode probe configured for implantation by insertion into soft tissue, in particular nervous, endocrine and muscle tissue, comprises an elongated conductive element having at least a proximal insulated portion and distal non-insulated portion, at least part of the element being disposed in a casing of an electrically insulating non-degradable material comprising a first structural components, the distal non-insulated portion of the element being encapsulated by the casing forming a distal chamber, a void/lumen being present between the insulated portion of the conductive element and the first structural component the void/lumen enabling the conductive element to slide with respect to the casing, the casing of the distal chamber comprising at least one electrically conductive bridge electrically coupling the distal chamber with the adjacent soft tissue, and the distal chamber comprising a distal matrix comprising a biocompatible materials providing sufficient rigidity to the probe when dry for insertion into soft tissue and dissolvable or degradable in aqueous body fluids

A further embodiment relates to a microelectrode configured to be at least partially embedded into or at least partially placed adjacent to soft tissue, in particular nervous, endocrine and muscle tissue, comprising an elongated electrically conductive element comprising a proximal and distal end, the electrically conductive element comprising insulated proximal and distal portions, the proximal insulated portion extending in distal direction from the proximal end, the distal insulated portion extending in proximal direction from the distal end, the proximal and distal portions separated by a non-insulated portion of the conductive element, at least the non-insulated portion of the conductive element being essentially centrally disposed in a casing of an electrically insulating non-degradable material, the microelectrode further comprising first and second structural components, said first and second structural components extending in radial direction between the casing and the conductive element, the first structural component movably disposed around the proximal insulated portion, the second structural component movably disposed around the distal insulated portion; the casing, first and second structural components forming a distal chamber, wherein the casing has an opening distally to the second structural component, wherein the casing is configured to electrically couple the non-insulated portion of the conductive element with the soft tissue, and where the distal chamber comprises a distal matrix comprising a biocompatible materials providing sufficient rigidity to the probe when dry for insertion into soft tissue and dissolvable or degradable in aqueous body fluids.

If the first structural component partitions the casing in a distal chamber and a proximal compartment, at least part of the proximal insulated portion of the conductive element being disposed within the proximal compartment, preferably the proximal compartment comprises a distal matrix comprising biocompatible materials providing sufficient rigidity to the probe when dry for insertion into soft tissue and dissolvable or degradable in aqueous body fluids.

The proximal and distal matrices may not be of the same material. Furthermore, the matrices, be it proximal and distal matrices or any other matrix of the probe or array, may comprise substances biologically active substances such as pharmacologically active substances and gene constructs. According to an embodiment, the distal matrix may comprise biologically active substances.

Biologically active substances are suitably selected from anti-inflammatory substances, neurotrophic substances, sedatives, transmitter substances such as glutamate, glycine, GABA, dopamine, noradrenalin, and acetylcholine or substances interfering with synaptic transmission. The pharmacologically active substances are suitably comprised within the distal chamber such that these substances can be released through opening(s) and or electrically conductive bridges comprising conduits in the distal chamber. The biologically active substance may during the manufacturing of the microelectrode probe be added to any of the matrices, such as distal, proximal, embedding, array embedding matrix, either to just one matrix, some of them or all of them. According to an embodiment, the biologically active substance is added to the surface of the distal matrix and/or is comprised in the distal matrix. Also, the biologically active substance may be applied on the conductive element, specifically to the distal non-insulated portion of the conductive element located within the distal chamber.

A drug may also be delivered to a targeted region of the soft tissue through a microelectrode comprising a hollow conductive element, i.e. conduction element comprising a central conduit. Any of the biologically active substances which can be comprised in a dissolvable and/or degradable array matrix may also be delivered to a targeted soft tissue region via a hollow conductive element. The casing of the microelectrode detached from the conductive element is to a significant degree associated with the soft tissue. Hence, the microelectrode comprising a hollow conductive element may serve as a catheter for a longitudinal delivery of drugs to a precisely targeted area of soft tissue. Additionally, the impact of the drug delivery on the soft tissue can be immediately electrically monitored enabling a drug dose regime attuned to immediate soft tissue feed-back.

According to an embodiment, the microelectrode probe may also comprise a further matrix embedding the microelectrode featuring distal and optionally proximal compartments comprising matrices. Such matrices embedding the microelectrode are referred to as embedding matrices.

The microelectrode or the microelectrode probe may also comprise a holder for the conductive element. In some embodiments, the holder can comprise a proximal casing wherein the conductive element can slide (e.g. a proximal bridging arrangement as disclosed herein). The holder for the conductive element preferably comprises or consists of a stiff material and comprises a distal face and a proximal face. It is preferred that a proximal terminal section of the proximal insulated portion of the conductive element penetrates the element holder from the distal to the proximal face. It is preferred for the holder for the conductive element to comprise a cylindrical tube of smaller diameter than that of the element holder, in particular of a diameter equal to or smaller than the diameter of the bore in a bone at which the element holder is to be mounted, the tube extending from a distal face of the element holder in a distal direction. The tube is of same material as the holder or of a different material and is stable against degradation by aqueous body fluid.

A further embodiment is related to an array of microelectrodes comprising a non-degradable continuous element, also referred to as non-degradable matrix, and/or non-degradable discontinuous elements such as micro- or nanofibers, the microelectrodes being adhesively attached to microfibers. Suitably, the non-degradable continuous element or micro- or nanofibers are capable over time to essentially maintain the mutual spatial positioning of the microelectrodes of the array when the microelectrode array is positioned adjacent to soft tissue or embedded in soft tissue. The array of microelectrodes comprising a non-degradable continuous element (non-degradable matrix) and/or non-degradable discontinuous elements may be disposed in a rigid array matrix of biocompatible material providing sufficient rigidity to the array when dry for insertion into soft tissue and dissolvable or degradable in aqueous body fluids.

Micro- or nanofibers for use in the invention are preferably essentially non-degradable or non-degradable. It is particularly preferred for micro- or nanofibers of the invention to be used in form of non-woven nano- or microfiber aggregates. Non-woven microfiber aggregates consist of irregularly intertwined microfibers and may comprise microfibers attached to each other in an irregular manner such as by attachment caused by local melting and/or by gluing with a biocompatible glue.

The non-degradable continuous element capable of essentially maintaining the mutual spatial positioning of the microelectrodes of an array when the microelectrode array is positioned adjacent to soft tissue or embedded in soft tissue over time is suitably a polymer matrix and/or non-degradable micro- or nanofibers adhesively attached to microelectrodes. Suitable non-degradable polymeric matrix materials (for the continuous element) are flexible biocompatible polymeric matrices selected from any one of the electrically insulating non-degradable flexible polymeric materials disclosed herein. Additional non-degradable polymers acting as non-degradable continuous element (non-degradable matrix) include polyurethanes, silicones, polymers comprising recurring benzene moieties (such as parylenes) and other suitable biocompatible flexible polymers essentially not degradable upon contact with soft tissue, and in general tissue fluid.

The time for positional stability by integration with the non-degradable continuous element and/or non-degradable discontinuous elements such as nano- and microfibers may range from at least several days up to at least to a couple of weeks, such as 2 or 5 weeks, and more preferable at least a few months to preferably at least several years.

Microfibers of the invention are in the micro- or nanometer diameter range. Particularly preferred are electrospun nano- and microfibers and electrospinning is a preferred method for producing microfibers of the invention.

A particularly preferred kind of microfibers are electro-spun microfibers. According to preferred aspect of the invention the microfibers form a non-woven irregular structure. It is preferred for a microfiber to be adhesively attached to a microelectrode and to one or more other microfibers. Preferably the microfibers are disposed along 50% or more of the axial extension of a microelectrode. Microfibers for use in the invention can be of a resilient or a non-resilient material.

Another aspect of the invention relates to processes for the manufacturing of the microelectrodes and microelectrode probes. Dependent on the design of the first structural component two different manufacturing processes are presented. FIGS. 6 to 16 disclose several manufacturing stages for the manufacturing of a microelectrode/microelectrode probe where the first structural component forms an integral part of the casing. FIG. 22 illustrates one stage of the manufacturing process where the first structural component is not integrated with the casing but is separate from the casing.

The invention encompasses a method for manufacturing the microelectrode, microelectrode probe or array, comprising:
providing an elongated electrically conductive element,
covering a proximal portion of the element with an electrically insulating layer thereby providing a proximal electrically insulated portion and a distal non-insulated portion of the conductive element;
forming a distal matrix dissolvable or degradable in aqueous body fluids extending axially around the distal non-insulated portion of the conductive element, and optionally extending in a distal direction from the distal non-insulated portion of the conductive element;
applying a sliding facilitating composition to a section of the insulated element proximally with respect to the distal matrix and distally with respect to an optional proximal matrix which sliding facilitating composition facilitating the axial movement of a first layer of electrically insulating non-degradable material with respect to the insulating layer of the conductive element, said medium optionally providing for a sufficient void/lumen between the insulating layer of the conductive element and first layer of electrically insulating non-degradable material;
optionally forming a proximal matrix extending axially around at least part of the proximal electrically insulated portion of the conductive element;
covering the distal matrix and at least part of the proximal electrically insulated portion of the conductive element with a first layer of electrically insulating non-degradable material, thereby providing a casing encapsulating the distal non-insulated portion of the element forming a distal chamber and a first structural component
cutting the non-insulated portion of the conductive element (preferably a part of the non-insulated portion of the conductive element) and first layer of electrically insulating non-degradable material near the distal end of the distal matrix comprising the distal non-insulated portion of the electrically conductive element, thereby providing a distal opening of the distal chamber.
applying a further distal tip matrix distally to the distal opening,
covering the tip matrix and at least part of the first layer with a second layer of electrically insulating non-degradable material and at least part of the first layer, thereby forming a distal end cap part forming part of the casing of the distal chamber
wherein the distal and optionally proximal matrices provide structural support to the microelectrode or probe when dry for insertion into soft tissue and;

Additionally, a section of the first layer and optionally second layer of the casing surrounding the distal matrix is removed by suitable means (e.g. laser milling) and an replaced with an electrically conductive bridge e.g. in form if a conductive mesh. Alternatively or additionally, conductive filaments may be positioned in the distal matrix protruding from the matrix essentially radially form the distal matrix. After the first layer is disposed on the distal matrix and optional a second layer disposed on the first layer, first and optional second layers of electrically insulating non-degradable material must be removed from the filaments such that parts of the filaments can be electrically coupled with soft tissue outside the distal chamber.

A further variant of the method for manufacturing the microelectrode, microelectrode probe, or array as disclosed herein comprises:
providing an elongated electrically conductive element;
covering a proximal portion of the element with an electrically insulating layer thereby providing a proximal electrically insulated portion and a distal non-insulated portion of the conductive element;
forming a distal matrix dissolvable or degradable in aqueous body fluid extending axially around, and optionally extending in a distal direction from the distal non-insulated portion of the conductive element;
forming a proximal matrix extending axially around at least part of the proximal electrically insulated portion of the conductive element and thereby forming an intermediate section of the insulated conductive element with an axial extension, the intermediate section positioned proximally to the distal matrix and distally to the proximal matrix not covered by the distal and proximal matrices;
applying a thin (up to about 5 μm) layer of a first intermediate matrix and/or sliding facilitating composition to the intermediate section of the insulated element facilitating the axial movement of a first layer of electrically insulating non-degradable material with respect to the insulating layer of the conducing element, said first intermediate matrix and/or composition providing for a sufficient void/lumen (annular channel) between the electrically insulated portion of the conductive element and the first layer of electrically insulating non-degradable material;
covering distal, proximal matrices and the intermediate section of the proximal electrically insulated portion of the conductive element, the intermediate section comprising an intermediate matrix and/or sliding facilitating composition, with a first layer of electrically insulating non-degradable material, thereby providing a casing comprising a distal chamber, a first structural component and a proximal compartment;
optionally providing a second intermediate matrix on the first layer of electrically insulating non-degradable material in the constriction in radial direction of the first layer between the distal chamber and proximal compartment;
cutting (part of) the distal non-insulated portion of the electrically conductive element and the first layer of electrically insulating material near the distal end of the distal matrix (distal end of the distal chamber), thereby providing a distal opening of the distal chamber;
applying a further distal tip matrix distally to the distal opening;
covering the distal tip matrix and at least part of the first layer with a second layer of electrically insulating material thereby forming a distal end cap forming part of the casing of the distal chamber;
and removing the first layer and optionally second layer at a circumferential annular zone of the proximal matrix;
wherein the distal matrix, distal tip matrix, proximal matrix and optionally first and second intermediate matrices are of a biocompatible material providing sufficient rigidity to the probe when dry for insertion into soft tissue and dissolvable or degradable in aqueous body fluids;

Additionally, a section of the first layer and optionally second layer of the casing surrounding the distal matrix is removed by suitable means (e.g. laser milling) and an replaced with an electrically conductive bridge e.g. in form if a conductive mesh.

Still a further embodiment of a method for manufacturing the microelectrode comprising:
providing an elongated electrically conducting element,
covering a proximal portion of the element with an electrically insulating layer thereby providing a proximal electrically insulated portion and a distal non-insulated portion of the element;
providing a first structural component configured to enable an axial movement with respect to the proximal electrically insulated portion of the conductive element;
positioning the first structural component around the proximal electrically insulated portion of the element, suitably at a certain axial distance from the distal non-insulated portion of the conductive element;
applying a proximal matrix dissolvable or degradable in aqueous body fluids around the proximal electrically insulated portion of the conductive element, the proximal matrix extending from the proximal face of the first structural component in proximal direction;
applying a distal matrix dissolvable or degradable in aqueous body fluids around the distal non-insulated portion of the conductive element extending from the distal face of the first structural component in distal direction, and extending in a distal direction from the distal non-insulated portion of the conductive element;
applying a first layer of electrically insulating non-degradable material on the proximal and distal matrices and the circumference of the first structural component, thereby forming a casing comprising a distal chamber and a proximal compartment;

Additionally, a section of the first layer of the casing surrounding the distal matrix is removed by suitable means (e.g. laser milling) and an replaced with an electrically conductive bridge e.g. in form if a conductive mesh.

Manufacturing of preferred embodiment E and D

The below example describes manufacturing of one variant in which cross linked hydrogels are used to provide a conductive inner casing. Instead of hydrogels other conductive materials can also be used.
1. provide a straight conductive element (for example platinum), insulate the element with a polymer such as Parylene C and de-insulate, by e.g. laser milling, a defined section of the conductive element, such that the conductive element remains insulated distal and proximal to the de-insulated section.
2. provide two electrically insulated tubes, of for example Teflon or Nylon, with an inner diameter larger than the outer diameter of the insulated conductive element. These pieces will are first and second structural components. It is preferred to provide the first and second structural components with transverse groves on their outer surfaces.
3. provide an inner casing, with an inner diameter somewhat larger that the diameters of the first and second structural components, equipped with one or more radial protrusions (with a length larger than the thickness of the casing). Cross-linked hydrogels, e.g. gelatine strongly cross-linked with glutaraldehyde, freeze dried in a maximally swollen state can be used as materials for the inner casing. Optionally, conductive bridge elements, e.g. hollow carbon tubes or hollow platinum tubes are incorporated into, or enwrapped around, the protrusion(s).
4. Align the first structural component, inner casing and second structural components. Insert the conductive element through the first structural component, inner casing and second structural component such that the insulated portion of the distal conductive element is covered by the secondary structural component and that the non-insulated portion of the conductive element is enclosed by the second casing. Optionally glue the parts together with a biocompatible glue that is dissolvable in body fluid for easy handling. Optionally coat the second casing with a dissolvable biocompatible matrix material such as glucose.
5. Coat the proximal insulated conductive element protruding in a proximal direction from the first structural component with a dissolvable matrix material and add a cone shaped distal tip of dissolvable matrix material to the distal part of the secondary structural component. Dipcoating or electrospraying are preferred method as the coating thickness can be easily controlled.
6. Provide a Parylene C coating of suitable thickness on the ensemble using standard methods.
7. Cut the Parylene C coating distal to the second structural component so as to open a hole in the cone shaped distal tip.
8. Cut and take away the Parylene C coating at a suitable distance proximal to the first structural component.
9. Cut the protrusion(s) from the distal chamber to provide hollow protrusion(s) of suitable length.

A conductive second casing in a hydrogel can be manufactured by dipcoating a suitably seized pin in a gelatine solution, subsequently crosslinking and swelling the hydrogel in water, freeze drying the hydrogel and removing the second casing from the pin.

Non-conductive first and second structural components can be made from Teflon coated stainless steel needles (of appropriate size) by removing the needles after Teflon curing.

As elaborated the method may comprise conductive bridges, such as electrically conductive bridges, e.g. electrically conductive bridge comprising at least one non-fluidic electrically conductive bridge, are applied to at least part of the surface of the distal matrix. The conductive bridges are suitably applied to the distal matrix that parts of the electrically conductive bridges are immersed by the distal matrix. Also, it is preferred that the conductive bridges are disposed with respect to the distal matrix that they can provide a conductive bridge between the distal chamber and the adjacent soft tissue through the distal casing (of electrically insulating material) once the microelectrode is operational inside soft tissue. The electrically conductive bridges are preferably disposed with respect to the distal matrix in mainly a radial direction.

If the microelectrodes or microelectrode probes comprise electrically conductive bridges and the method comprises one or more steps eventually covering the electrically conductive bridges with electrically insulating material the method preferably comprises a step providing that the electrically conductive bridges once the microelectrodes is operational and disposed in soft tissue electrically couple the distal chamber with the adjacent soft tissue.

As alluded to above the electrically conductive bridges may comprise a central conduit, the electrically conductive bridges may be hollow. Should the electrically conductive bridges be hollow it is preferred to apply a further coat of dissolvable matrix after the application of the electrically conductive bridges to the distal matrix such that the central conduit is filled with dissolvable matrix. Once, first and/or second layers of electrically insulating non-degradable material have been applied the tips of the hollow electrically conductive bridges partially filled with dissolvable matrix can be cut thereby providing that the dissolvable matrix of the hollow electrically conductive bridges is in fluid contact with soft tissue fluids once the microelectrode is inserted into soft tissue. The soft tissue fluid will gradually dissolve the dissolvable matrix thereby providing hollow electrically conductive bridges enabling migration of charges particles between the distal chamber and the adjacent soft tissue as well as providing an electrical bridge between the distal chamber and adjacent soft tissue.

According to an aspect, the proximal matrix widens in a proximal direction.

An array of microelectrodes, e.g. sheet-formed array, where the microelectrodes are disposed essentially in parallel and the microelectrodes being attached to a non-degradable matrix and/or adhesively attached to non-degradable micro- or nanofibers may be produced by providing a template mimicking the form of the soft tissue adjacent to which the array is to be positioned. The microelectrodes are disposed essentially in parallel (with respect to the microelectrodes) on the template with due account that the openings and/or electrically conductive bridges are positioned such that they engage with the soft tissue once the sheet-formed array is disposed adjacent to soft tissue. The non-degradable matrix and/or non-degradable micro- or nanofibers is (are) either applied on the template prior or after positioning the microelectrodes on the template. It is preferred that the microelectrodes are attached to the non-degradable matrix and/or non-degradable micro- or nanofibers along part of their axial extension, suitably along a distal section of their axial extension which allows the conductive elements proximally to the non-degradable matrix to be bundled together.

### SHORT DESCRIPTION OF THE FIGURES

FIG. 1A region of neural tissue for implantation of a microelectrode probe of the invention, in a section perpendicular to a bone protecting the region

FIG. 2 The region of FIG. 1 after providing a circular hole in the bone, in the same section

FIG. 3 An electrode according to FIG. 3a immediately upon implantation

FIG. 3a A schematic representation of a microelectrode probe of the invention in an axial section

FIG. 4A microelectrode of the invention with a plurality of electrically conductive bridges the distal chamber

FIG. 4b A microelectrode comprising a hollow conductive element

FIG. 4c A microelectrode with a distal chamber comprising electrically conductive bridges

FIG. 5A microelectrode of the invention with a distal chamber but without a proximal compartment proximally to the distal chamber

FIG. 5a A microelectrode of the invention featuring a tubular structure distinct from the casing.

FIG. 5b A microelectrode of the invention featuring a tubular structure distinct from the casing further comprising a structural element within distal chamber

FIG. 6-14 A process for the manufacturing of a microelectrode probe of the invention showing consecutive pre-stages to the microelectrode probe illustrated in FIG. 16

FIG. 15 Microelectrode probe of the invention in axial direction

FIG. 16A variety of a microelectrode of the invention comprising an embedding matrix

FIG. 17A microelectrode probe of the invention implanted in neural tissue prior to the dissolution of embedding matrix and proximal and distal matrices

FIG. 18A proto microelectrode of the invention implanted into neural tissue in a state of partial dissolution of the embedding matrix and in a stage of transformation to a microelectrode of the invention

FIG. 19A microelectrode of the invention formed in situ (in situ microelectrode) from the microelectrode probe of FIG. 17

FIG. 19a A microelectrode of the invention formed in situ (in situ microelectrode) from the microelectrode probe of FIG. 17. The casing has accommodated for spatial movement of the surrounding soft tissue.

FIG. 20 An array of four microelectrode probes of the invention

FIG. 20a A sheet-formed array with microelectrodes disposed essentially in parallel

FIG. 20b A cuff-like sheet-formed array with microelectrodes disposed essentially in parallel

FIG. 21A tubular cross section of the array through the distal chambers of the microelectrode probes

FIG. 22 Half mold with tubular structure of a manufacturing step for producing a microelectrode featuring a tubular structure distinct from the casing

FIG. 23 Tubular structure comprised in variants of the microelectrode

FIG. 24A variant of the microelectrode of the invention where the radial extension of the casing of the distal chamber is only marginally wider that the radial extension of the insulated portion of the conductive element.

FIG. 25 An array of microelectrodes. The individual microelectrodes are held together by a web of micro- or nano-fibers.

FIG. 26A microelectrode comprising an engaging element. The casing also exhibits micro- or nano-fibers increasing the friction of the casing with respect to the surrounding soft tissue.

FIG. 27A microelectrode or proximal bridging arrangement according to a specific embodiment.

FIG. 28A microelectrode where part of the casing of the distal compartment is replaced with a lateral member.

FIG. 29a/b/c/d

Cross-cut views at A-A of the microelectrode of FIG. 28 illustrating several embodiments of the radial extension of the lateral member.

FIG. 30A microelectrode comprising a hollow conductive element similar to FIG. 4b but comprising a lateral member instead of holes.

FIG. 31A microelectrode with a distal chamber comprising a lateral member, the first structural member forming at integral part of the casing.

FIG. 32A cross-sectional view of a microelectrode comprising first and second structural components, the non-insulated portion of the conductive element disposed in an inner casing, the distal casing comprising an opening distally to the second structural component.

Several embodiments of the invention are described in more detail below. The embodiments should not be construed as to limit the general concept of the invention.

### DESCRIPTION OF SOME EMBODIMENTS

Implantation and tissue environment principles.

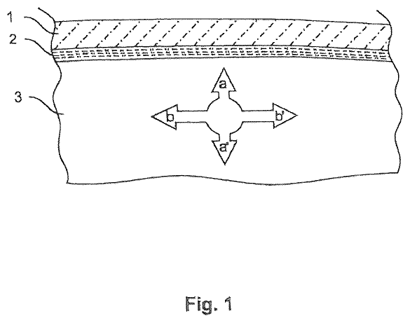

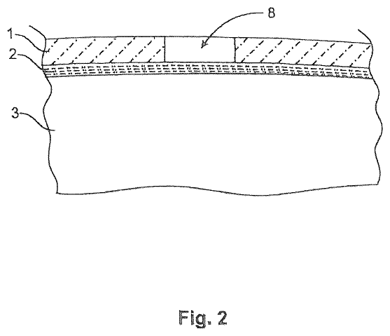

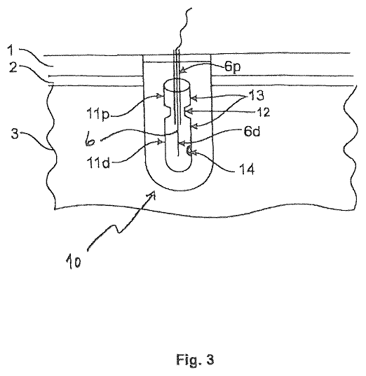

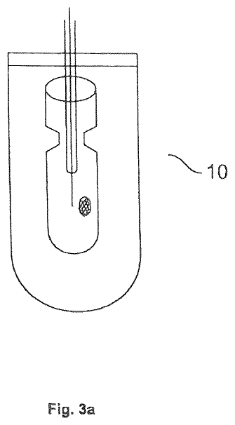

FIGS. 1, 2, 3a and 3 illustrate schematically the intersection of a skull without a microelectrode (FIG. 1 and FIG. 2), an implanted microelectrode probe into neural tissue (FIG. 3) and a microelectrode probe (FIG. 3a). The neural tissue (3) here is brain tissue, protected by the skull bone (1) from which it is separated by meninges (2) comprising several sub-layers, such as the dura mater, the arachnoid, the pia mater and cerebrospinal fluid. The neural tissue (3) is prone to spatial displacement in respect of the skull bone (1) by movements of the head, the displacement schematically depicted in direction parallel with the skull bone (1) (arrows b, b′) and perpendicular direction (arrows a, a′). Tissues (2) intermediate between the skull bone 1 and brain tissue 3 are also displaced somewhat in relation to the skull and brain during such movement.

Prior to implantation of a device according to the invention access to a desired position of the brain is provided by drilling a hole (8) in the skull (FIG. 2) and optionally providing an opening in the dura mater using for example a diamond knife.

In the next step a device of the invention, such as the microelectrode probe (10) of the invention of FIG. 3a or a microelectrode probe array, is inserted through the hole (8) into brain tissue (3) (FIG. 3). Upon implantation the microelectrode probe (10) is transformed into a microelectrode (in situ microelectrode) of the invention by contact with aqueous body fluid. The fully functional in situ electrode is formed once the matrix materials have completely dissolved or been degraded. The microelectrode probe (10) comprises a cover (7) anchored in the skull bone at the hole (8) protecting the skull bone and soft tissue. The microelectrode (10) comprises a metallic or other electrically conductive element (6) attached to and penetrating the cover (7), which extends from the proximal face of the cover (7) for electrical communication with a microelectrode control unit (not shown) disposed extracorporeally or implanted under the skin. A proximal portion of the conductive element (6p) is electrically insulated while a distal portion of the conductive element (6p) is non-insulated. A first structural component (12) divides the casing (13) into a proximal compartment (11p) and a distal chamber (11d). The distal chamber is encapsulated by the casing further comprising an electric conductive bridge in form of a conductive lateral mesh member (14) enabling an electric current to flow between the distal non-insulated portion of the conductive element (6d) and the neural tissue (3). In this microelectrode the first structural component is integrated with casing. The first structural component forms an integral part of the casing. Hence, the casing and the first structural component share the same material. The casing, i.e. first structural component, is slidably connected to the proximal insulated portion of the conductive element (6p).

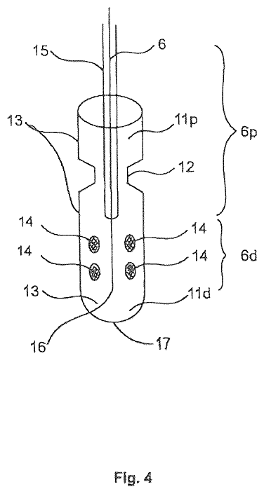

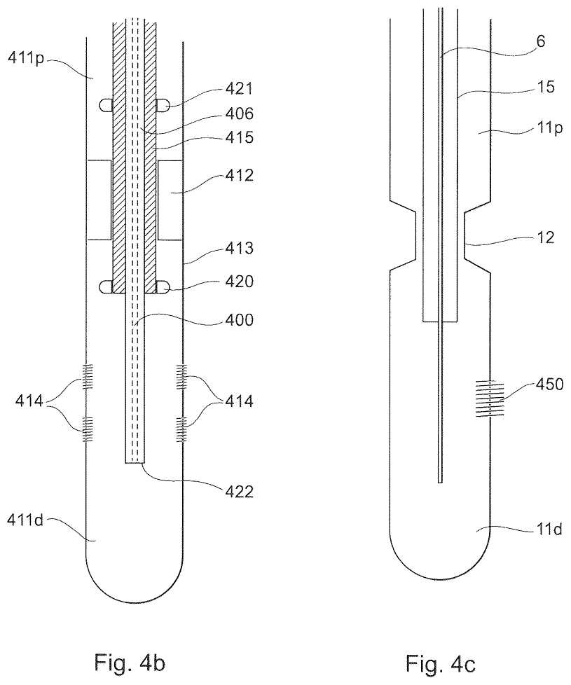

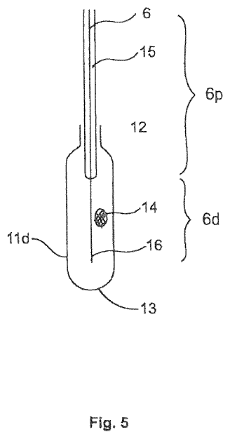

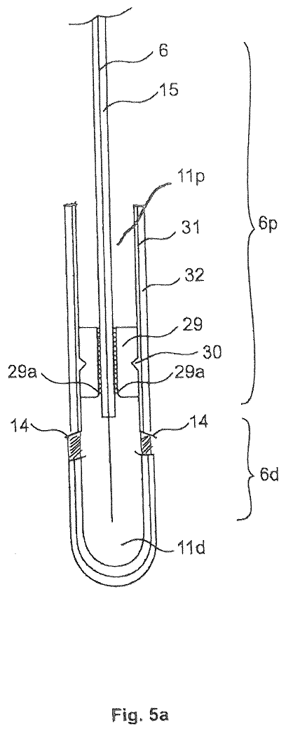

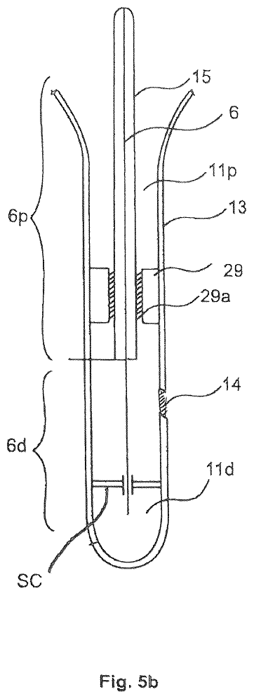

FIGS. 4, 5, 5a and 5b show four variants of the microelectrode as configurated after complete dissolution of matrices.

FIG. 4 shows a variant of the microelectrode as configurated after complete dissolution of the matrices of biocompatible material dissolvable or degradable in aqueous body fluids. This variant comprises a proximal (11p) compartment and a distal chamber (11d). Between the proximal compartment and distal chamber, a first structural component (12) is present embracing the proximal insulated portion (6p) of the conductive element (6). As seen in FIG. 4 the casing (13) encapsulates the distal chamber (11d). The first structural component (12) embracing the insulated portion of the conductive element (6p) is slidably attached to the outermost layer of the proximal insulated portion of the conductive element. Here, the outermost layer is equivalent to the insulating layer (15) of the proximal portion of the conductive element (6p). Instead of one opening the distal chamber has four electric conductive bridges in from of conductive lateral mesh members (14). All four openings are axially positioned such that the perpendicular distance of the distal non-insulated portion of the conductive element (6d) to the openings remains essentially constant when the conductive element (6), i.e. distal non-insulated portion of the conductive element (6d) and proximal insulated portion of the conductive element (6p), moves with respect to the first structural component (12) which coincides with the movement of the conductive element with respect to the casing encapsulating the distal chamber. The distal tip (16) of the non-insulated conductive element should have enough travel distance in axial direction that the tip never penetrates the casing of the distal end cap (17) of the casing of the distal chamber (11d).

FIG. 4b shows an embodiment of the microelectrode comprising a hollow conductive element. In addition to a hollow conductive element the microelectrode also comprises a third and fourth structural elements restricting axial movement of the conductive element, specifically the insulated portion of the conductive element. FIG. 4b illustrates a hollow conductive element (406) comprising a central conduit (400) providing liquid communication between the distal chamber (411d) and a reservoir (not shown) comprising a drug. The distal chamber (411d) comprises an electric conductive bridge in form of a conductive lateral mesh member (414). The first structural component (412) is distinct from the casing (413). A proximal portion of conductive element is insulated (415). Third (421) and fourth (420) structural elements are disposed around the insulated portion of the conductive element. The third structural element (421) prohibits the tip of the non-insulated conductive element (422) from penetrating the casing of the distal chamber (411d). The fourth structural element (420) enables the removal of the casing when the microelectrode is removed from soft tissue by engagement of the conductive element located proximally and outside of the soft tissue.

FIG. 4 c illustrates an embodiment of the microelectrode comprising electrically conductive bridges (450) electrically coupling the distal chamber (11d) and the adjacent soft tissue. The first structural component forms an integral part of the casing (12). Also illustrated are the proximal compartment (11p), conductive element (6) and insulation of the conductive element (15).

FIG. 5 depicts a microelectrode variant comprising only a distal chamber (11d) encapsulating the distal non-insulated portion (6d) of the conductive element (6). The casing gradually transforms into a first structural component (integrated tubular structure) (12), the first structural component (12) being slidably attached to the proximal electrically insulated portion (6p) of the conductive element. The proximal insulated portion (6p) of the conductive element has an electrically insulating layer (15). The casing of the distal chamber comprises an electric conductive bridge in form of a conductive lateral mesh member (14). The an electric conductive bridge in form of a conductive lateral mesh member (14) is located axially such that the perpendicular distance of the opening (14) with respect to the non-insulated portion of the conductive element (6d) remains essentially constant even if the conductive element (6), i.e. the non-insulated portion of the conductive element (6d), moves in axial direction.

FIG. 5a shows a variant of the microelectrode comprising a first structural component (29) which does not form part of the casing (material) (31), (32). The first structural component which may be of Teflon® comprises a channel which accommodates the proximal insulated portion of the conductive element (6p). The first structural component features a recess (30) which may reach around the whole circumference of the first structural component. The recess secures the attachment of the casing (31) to the first structural component. The void/lumen (annular channel) (29a) between the proximal insulated portion of the conductive element and the first structural component is sufficient for the proximal insulated portion of the element to slide with respect to the first structural component. The casing comprising 1^{st} layer (31) and 2^{nd} layer (32) can be of Parylene C. Alternatively, 1^{st} (31) and 2^{nd} layers (32) can be made of different material. The 2^{nd} layer (32) may be of a material different from the material of the 1^{st} layer. Said 2^{nd} layer (32) may be a layer which exhibits increased friction with respect to the surrounding soft tissue compared to the material of the 1^{st} layer. Alternatively, or additionally, the outer surface of the 2^{nd} layer may exhibit a friction inducing surface structure. The casing comprises lateral electric conductive bridges in form of a conductive lateral mesh members (14).

FIG. 5b illustrates a variant sharing many of the design elements of the microelectrode of FIG. 5a with a difference that a second structural component (SC) is situated within the distal chamber (11d). The second structural component stabilizes the distal non-insulated portion (6d) of the element in radial (lateral) direction. Even if the soft tissue surrounding the microelectrode would move extensively displacing the casing extensively with respect to the element the second structural component (SC) stabilizes the radial movement of the distal non-insulated portion (6d) of the element resulting that the perpendicular distance between the distal non-insulated portion 6d of the element and the electric conductive bridge in form of a conductive lateral mesh member 14 remains similar over time.

### Manufacture of a Microelectrode of the Invention.

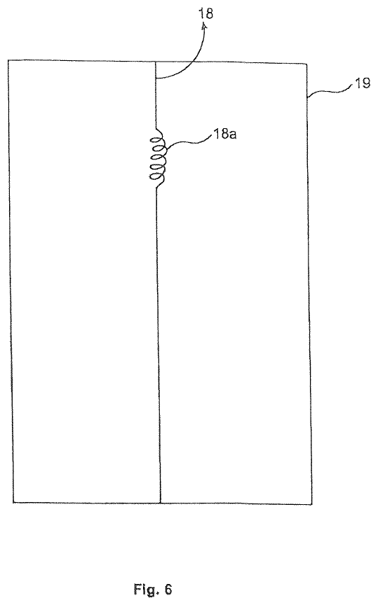

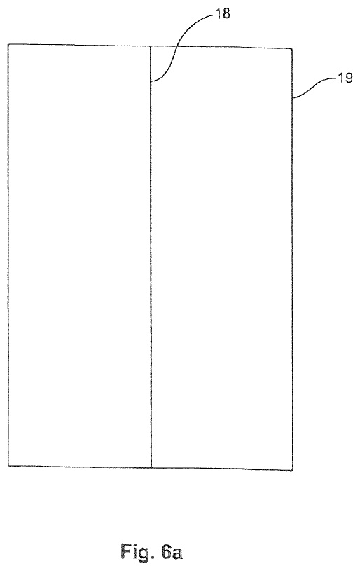

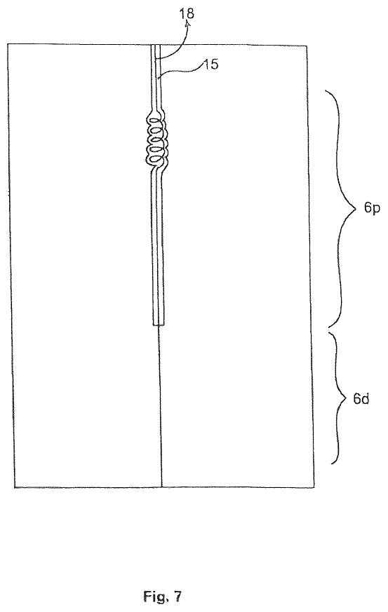

FIGS. 6 to 16 show several consecutive steps of one method of manufacturing of a microelectrode probe featuring a first structural component integrated with the casing.

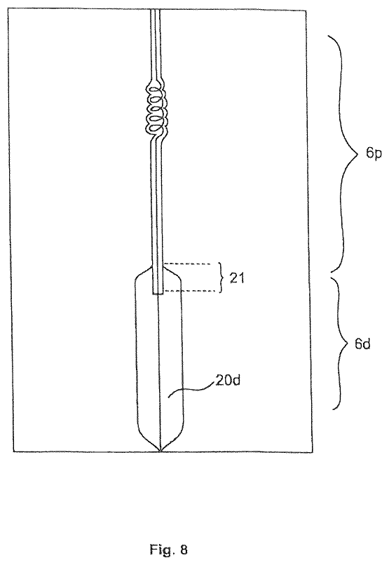

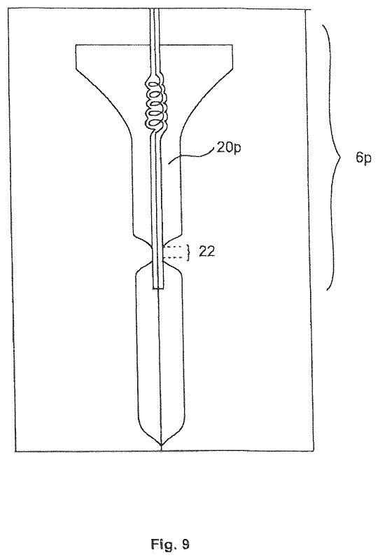

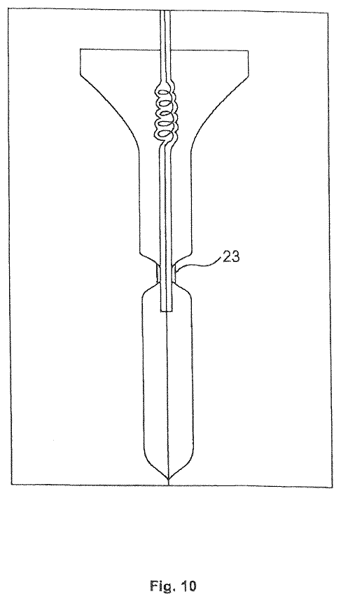

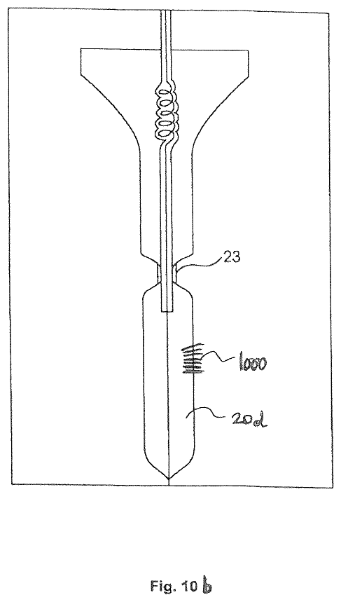

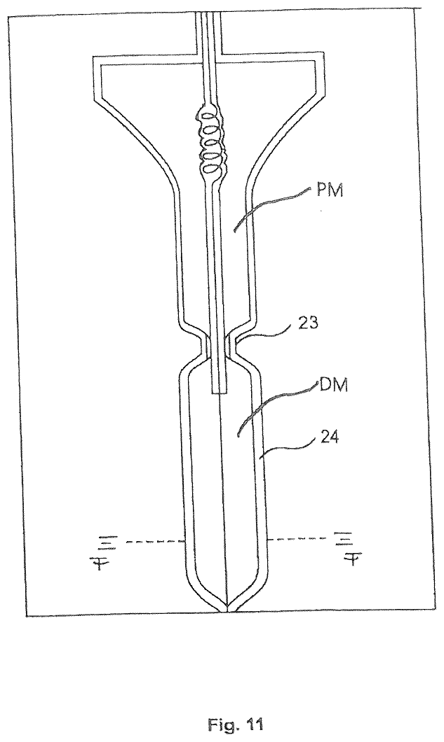

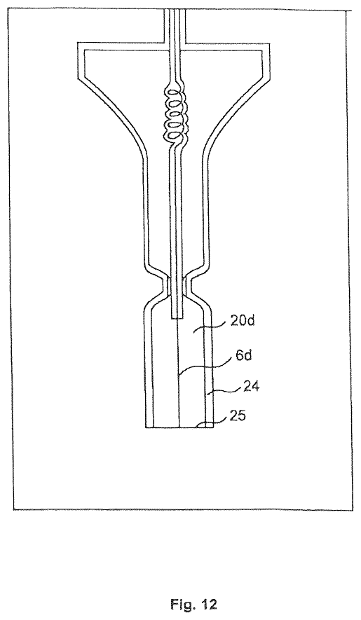

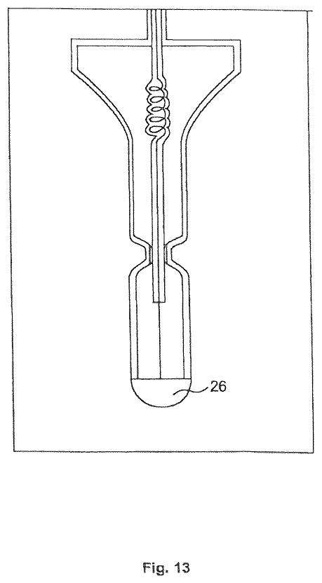

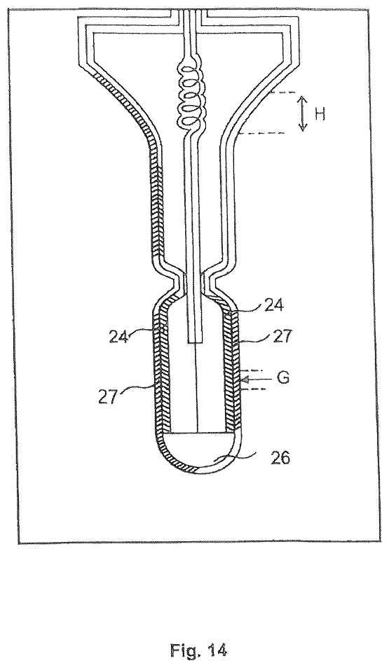

A metallic filament (conductive element) (18) is fastened at both ends to a frame (19). The metallic filament comprises a section (18a) which specifically enables the filament to flex in axial direction (FIG. 6) thereby allowing stretching of the conductive element. FIG. 6a shows a frame (19) with a conductive element (18) which does not comprise a section enabling the element to flex in axial direction. In a subsequent step a portion (6p) of the filament is covered with an electrically insulating non-degradable material (15), thereby forming the proximal insulated portion of the conductive element (6p). A distal portion of the conductive element (18) is not covered (6d) thereby providing the prerequisite for forming a distal non-insulated portion of the conductive element. Alternatively, insulation of the conductive element may be removed by evaporation. Next (FIG. 8) a distal matrix (20d) degradable (dissolvable in body fluids is formed radially around the distal portion of the non-insulated conductive element and part of the distal section (21) of the proximal insulated portion of the element. It is important that the matrix also covers part of the proximal insulated portion of the element (21). In FIG. 9 a proximal matrix (20p) is applied radially around part of the proximal insulated portion of the element (6p). An intermediate section (22) remains uncovered by matrix or preferably a thin layer of matrix of biocompatible material that is dissolvable or degradable in aqueous body fluids or other composition/substance, such as a composition facilitating the movement of the first structural component with respect to the insulated portion of the conductive element (FIG. 10: 23) is applied to the intermediate section around the element defining a void/lumen (annular channel) (23) between a 1^{st} layer of electrically insulating non-degradable material (such as parylene C) (24) (FIG. 11) and the insulated portion of the conductive element. FIG. 10b illustrates electrically conductive bridges (1000) disposed with respect to the distal matrix (20d) such that the bridges are partly immersed into the distal matrix. If a matrix or composition/substance is applied around the intermediate section of the proximal insulated portion of the conductive element such composition/substance may also facilitate axial movement of the casing (first structural component) and/or modulate the electric impedance between the proximal compartment and distal chamber. FIG. 11 shows a 1^{st} layer of electrically insulating non-degradable material (24) applied to the distal matrix (DM), intermediate section, and proximal matrix (PM). In a further step (FIG. 12) the non-insulated conductive element (6d), distal matrix (20d) and 1^{st} layer (24) are cut radially at a section F-F (FIG. 11) whereby a distal opening (25) is formed which in a subsequent step (FIG. 13) is covered by a distal cap (tip) matrix (26) of a spherical form. FIG. 14 depicts a 2^{nd} layer of electrically insulating non-degradable material (27) covering the distal cap matrix (26) and 1^{st} electrically insulating layer of electrically insulating non-degradable material (24). An electric conductive bridge in form of a conductive lateral mesh member (14) (FIG. 15) is provided through the casing encapsulating the distal chamber at an allocation G (FIG. 14). Furthermore, 1^{st} and 2^{nd} electrically insulating layers (24, 27) are removed around a circumferential band of height H forming an annular zone (28, FIG. 15) not covered by electrically insulating non-degradable material. The opening may be accomplished by laser evaporation and optionally followed by laser milling evaporation (FIG. 15).

The positioning and axial extent of the circumferential band may vary dependent on the types of tissues to be penetrated by the microelectrode probe.

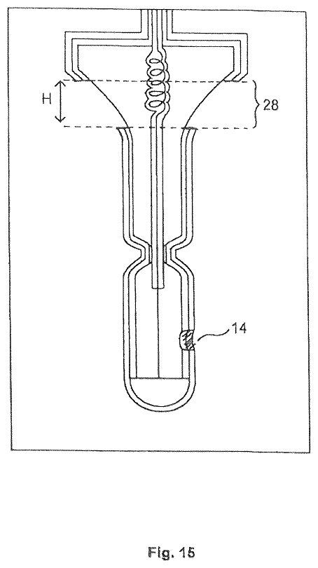

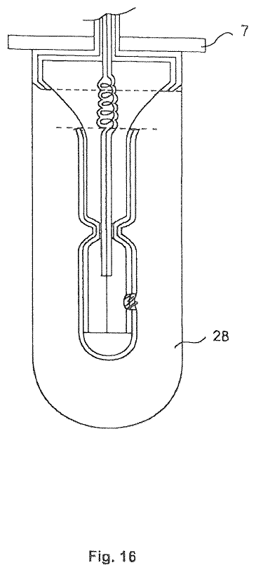

The opening (or openings) is/are preferably positioned axially with respect to the non-insulated element such that the (perpendicular) distance between the non-insulated element and the opening(s) remain(s) essentially similar when the non-insulated element moves axially. In a final step (FIG. 16) the proto microelectrode is covered by an embedding matrix (28) of biocompatible material dissolvable or degradable in aqueous body fluids. The embedding matrix can be formed by spray coating gelatin in a dry atmosphere. The microelectrodes of FIGS. 15 and 16 are both suitable to be inserted into soft tissue. Hence, FIGS. 15 and 16 present microelectrode probes. FIG. 16 also illustrates a cover (7) attached to the proximal face of the casing, the casing formed by 1^{st} and 2^{nd} electrically insulating layer of an electrically insulating non-degradable material. 1^{st} and 2^{nd} electrically insulating layer are preferably of Parylene C.

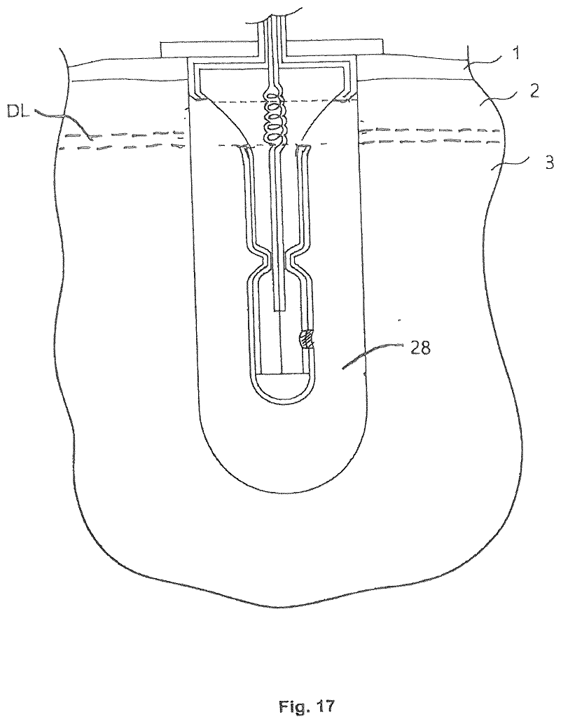

FIGS. 17 to 19 depict the microelectrode probe in various states after introduction into soft tissue (3) such as brain tissue. FIG. 17 presents the microelectrode probe immediately after inserted into brain tissue (3) through the skull bone (1) and tissue (2) intermediate between the skull bone such as dura mater, arachnoid membrane, cerebrospinal fluid, and pia mater (1) and brain tissue (3) (neuronal tissue) and prior to the dissolution of matrices. The two discontinued lines DL illustrate tissue regions which may have different characteristics as to e.g. the tendency for spatial movement (2 and 3).

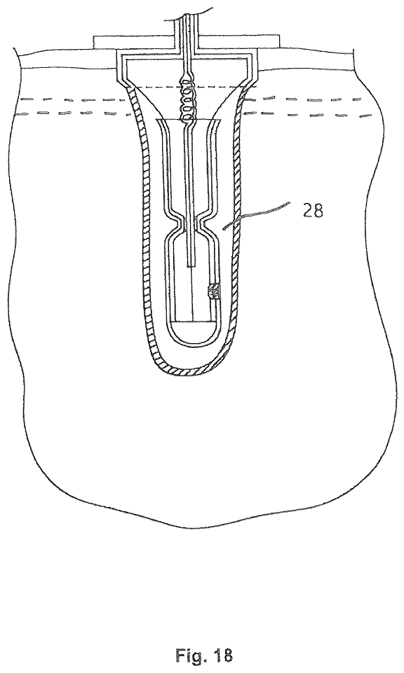

FIG. 18 indicates a partial dissolution of the embedding matrix (28).

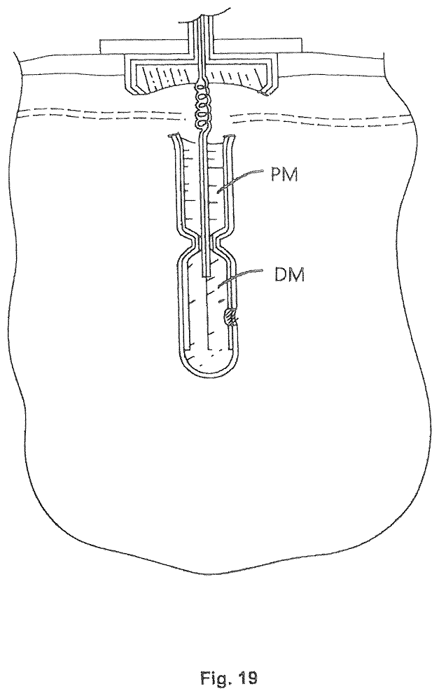

FIG. 19 illustrates a state of the microelectrode probe after complete dissolution of the embedding matrix and partial dissolution of distal (DM) and proximal (PM) matrices.

FIG. 19a is an example of a configuration of a microelectrode after complete dissolution of all matrices showing spatial movement of surrounding soft tissue. The casing (13) which may comprise 1^{st} and 2^{nd} electrically insulating layers of electrically insulating non-degradable material has attached (associated) to the surrounding soft tissue at a degree for being able to accommodate to the spatial movements of the soft tissue. The microelectrode also comprises a structural component SC stabilizing the movement of the non-insulated distal portion of the element (6d). The structural component SC is configured such that the distal portion of the element 6d can move in axial direction without much friction, yet, stabilizing the distal proportion sufficiently radially (laterally) that the (perpendicular) distance between distal non-insulated portion of element with respect to the electric conductive bridge in form of a conductive lateral mesh member 14 remains essentially same. Once the casing has attached to the surrounding tissue the opening of the casing communicates with essentially the same region of the soft tissue over time even when the soft tissue is moving.

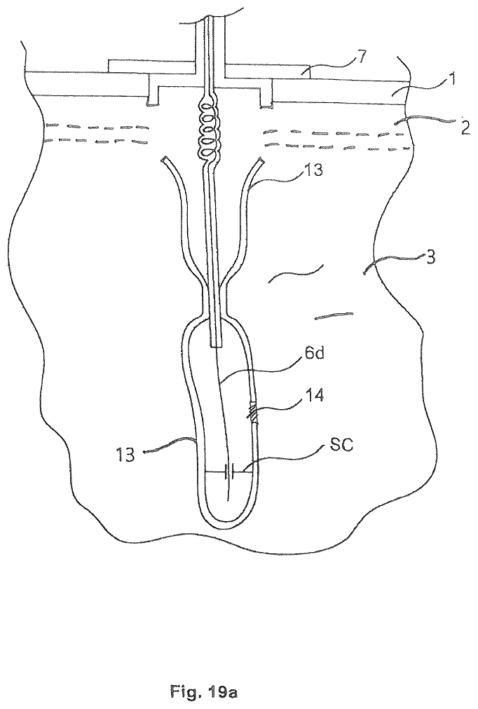

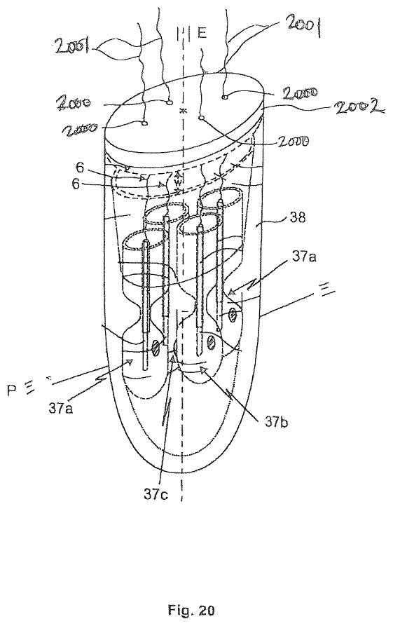

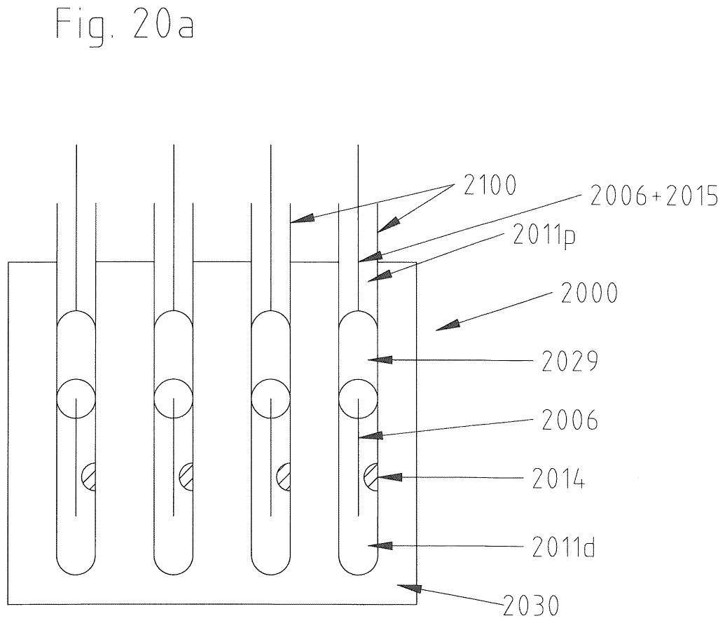

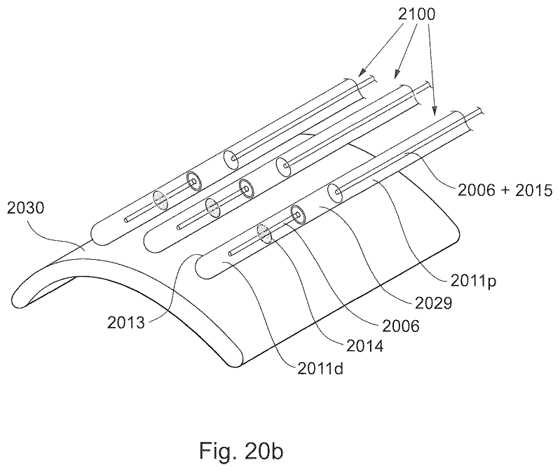

FIG. 20 illustrate an array of four microelectrodes (37a), (37b), (37c), (37d). The microelectrodes are embedded in an array matrix (38). Also illustrated are insulated conductive elements (6) attached to a cover (2002), the cover comprising microcontacts (2000) to which conductive elements (2001) are connected.

FIG. 20a illustrates a sheet-formed array (2000) of microelectrodes (2100) disposed essentially parallel. The microelectrodes comprise an insulated conductive element (2006+2015) and a non-insulated portion of the conductive element (2006). The microelectrodes comprise distal chambers (2011d) and proximal compartments (2011p). The distal chambers comprise electric conductive bridges in form of a conductive lateral mesh members (2014). The first structural component (2029) is distinct from the casing separating the casing into a distal chamber (2011d) and a proximal compartment (2011p). The microelectrodes are attached to a non-degradable matrix material (2030). The openings of the distal chambers are positioned such that they are not embedded into the non-degradable matrix material which secures that the openings engage with the soft tissue once the sheet formed array of microelectrodes is positioned adjacent soft tissue.

FIG. 20b illustrates a sheet-formed array having a cuff-like configuration. The array comprises a non-degradable matrix material (2030) attaching microelectrodes (2100) comprising insulated conductive elements (2006+2015), non-insulated portions of the conductive element (2006), first structural components (2029) dividing the casing (2013) into distal chambers (2011d) and proximal compartments (2011p). The distal chambers comprise electric conductive bridges in form of a conductive lateral mesh members (2014). The electric conductive bridges in form of a conductive lateral mesh members are not embedded by the non-degradable matrix material and are arranged such that they can engage with a peripheral nerve once the cuff-shaped sheet-formed array is positioned adjacent a peripheral nerve.

FIG. 21 illustrates a cross-section of an array at allocation P showing the array matrix (38), a casing encapsulating a distal chamber (39) and a distal non-insulated portion of the conductive element (40).

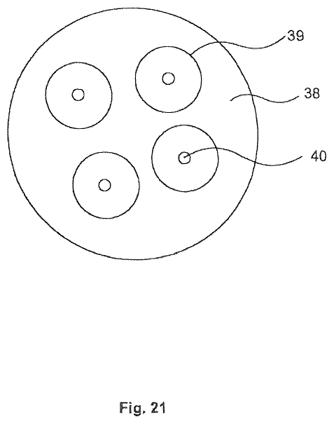

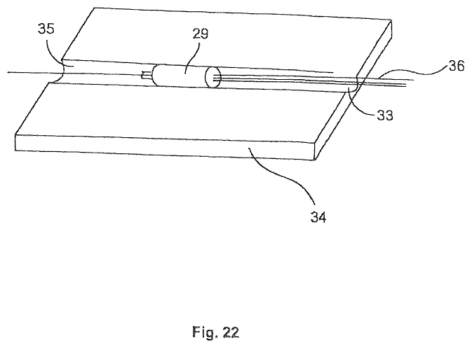

FIG. 22 illustrates a manufacturing step in the manufacturing of a microelectrode with a first structural component (29) of a different material than the casing. The first structural component is positioned around the proximal insulated portion of a conductive element (36) and placed within one first half of a mold (34) of silicone. The second half of the mold properly is positioned with regard to the first half of the mold. Before casting the proximal and distal matrices it is preferred to position the element centrally with respect to the mold.

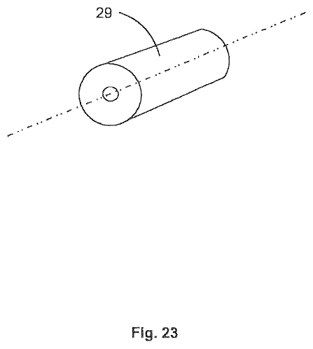

FIG. 23 shows a perspective view of first structural component (29) and the central axis as a dashed line.

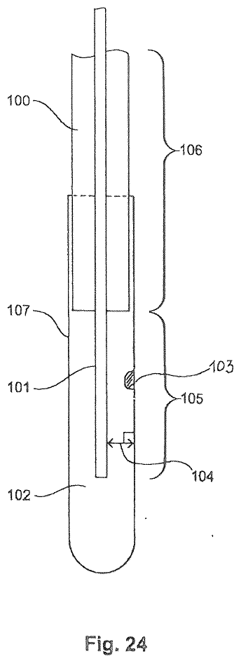

FIG. 24 shows a variant of the microelectrode comprising a conductive element (101). A proximal portion of the conductive element (106) is insulated with an electrically insulating non-degradable material (100) while a distal portion of the conductive element is non-insulated (105). A casing (107) of flexible electrically insulating non-degradable material encapsulates the non-insulated portion of the conductive element (105) forming a distal chamber (102). The casing of the distal chamber comprises an electric conductive bridge in form of a conductive lateral mesh member (103). The inner radial extension of the casing is such that it provides a void/lumen (108) between the casing and the insulated portion of the conductive element (106) for enabling an axial movement of the casing with respect to the conductive element. The numeral (104) visualizes what is meant by the perpendicular distance between the non-insulated portion of the conductive element (105) and the opening (103).

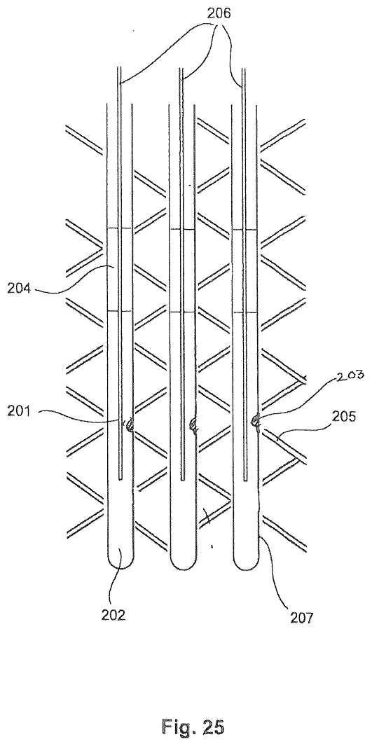

FIG. 25 presents a first array of microelectrodes attached to one another by micro- or nano-fibers (205). The conductive element (206), first structural components (204), casing (207), distal chambers (202), and an electric conductive bridge in form of a conductive lateral mesh member in the casing of the distal chambers (203) are shown. For reasons of simplicity, the insulation of the conductive elements are not indicated. An array of microelectrodes attached to one another by micro- or nano-fibers preferably having an extension providing a patch. The individual microelectrodes may be arranged essentially parallel in essentially one plane combined forming an array exhibiting a patch-like global extension. This type of array may be applied for monitoring and/or stimulating spinal nervous tissue.

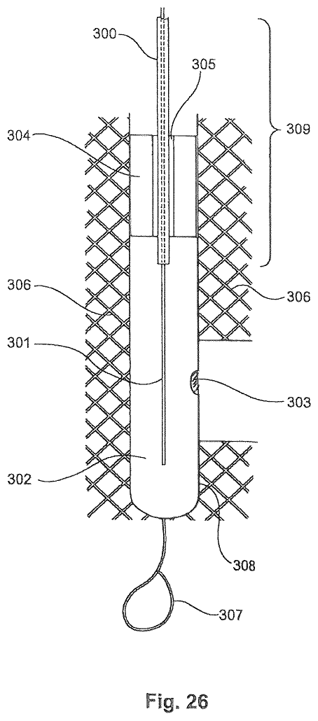

FIG. 26 shows a variant of a microelectrode comprising an engagement element (307). The casing (308) exhibits a net of micro- or nano-fibers (306) which preferably are adhesively attached to the external surface of the casing. The micro- or nano-fibers increase friction of the casing with respect to the surrounding soft tissue. FIG. 26 also presents a void/lumen (annular channel) (305) between the first structural component (304) and the insulation (300) around the conductive element (301) and surrounding the insulated portion of the proximal conductive element (309). For reasons of clarity the dimensions of the void are exaggerated. The engagement element is configured to reversibly engage with an elongated rigid pin such as a needle (not shown). The pin is further configured to insert the microelectrode into the soft tissue or placing the microelectrode adjacent to soft tissue. The casing shows an electric conductive bridge in form of a conductive lateral mesh member (303).

FIG. 27 illustrates a further embodiment based on the principle of having a casing (2706, 2704) detached from the conductive element (2701). The microelectrode comprises a conductive element (2701) which is insulated (2702) along an axial extension (2708). The insulated portion of the conductive element (2708) is slidably associated to a metal casing (2704) via a first structural component (2705). The dimensions of the first structural component (2705) or the insulated portion of the conductive element (2708) are configured such that an annular void is present between the first structural component (2705) and the insulated portion of the conductive element (2708). The metal casing (2704) is attached to the first structural component (2705). Furthermore, the metal casing (2704) is electrically insulated from surrounding body fluid by a layer of electrically insulating flexible material, such as parylene C. An insulated conductive wire is electrically connected to the metal casing connecting the microelectrode with suitable electronics for recordings and/or stimulation. A structural element (2703) is disposed at the proximal end of the insulated portion of the conductive element. This structural element (2703) prohibits the conductive element from separating form the casing (2704). The non-insulated distal portion (2709) of the conductive element (2701) is in electrical contact with the adjacent soft tissue.

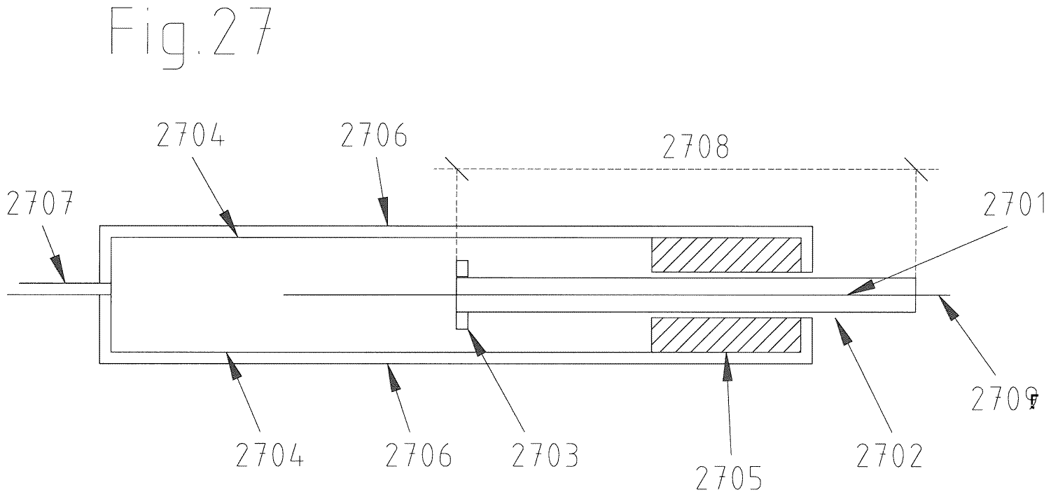

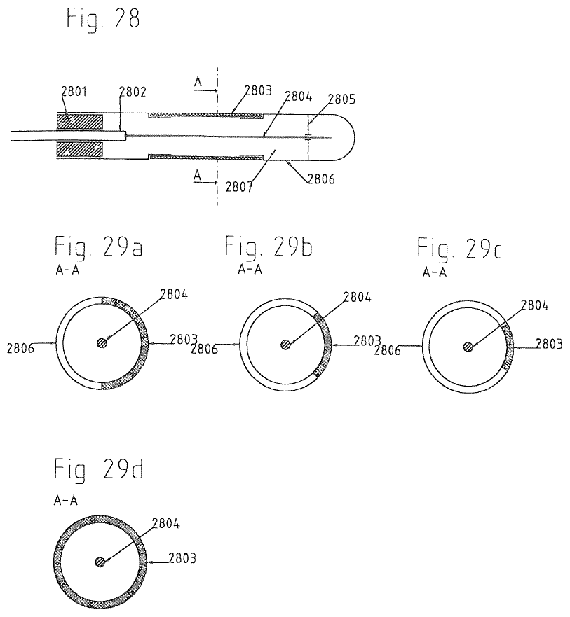

FIG. 28 illustrates a microelectrode comprising a conductive bridge in form of a lateral member of the casing of the distal chamber. The lateral member is a net of a conductive material (2803). The net of conductive material radially extends around the conductive element (2804). The microelectrode illustrated by FIG. 28 comprises a first structural component (2801) not forming part of the casing (2806). The first structural component is abutted to the casing and slidably disposed around an electrically insulated portion (2802) of the conductive element (2804). The casing around the electrically conductive element forms a distal chamber (2807). Also present in the microelectrode is a second structural component (2805) stabilizing the electrically conductive element in lateral direction. The casing (2806) which is not a lateral member constitutes of an electrically insulating, non-degradable material, such as parlyene C.

FIG. 29a/b/c/d, (2804) illustrate different embodiments of the microelectrode depicted by FIG. 28. More specifically, FIG. 29a/b/c/d illustrate cross-sectional views at A-A of FIG. 28. The embodiments of FIG. 29a/b/c/d illustrate microelectrodes comprising a lateral member having different radial extensions. Illustrated are radial extensions of the lateral member of 360°, 180°, 90° and 60°. (2806) illustrates the casing made of an electrically insulating, non-degradable material, while (2803) illustrates the lateral member in the form of a net of conductive material.

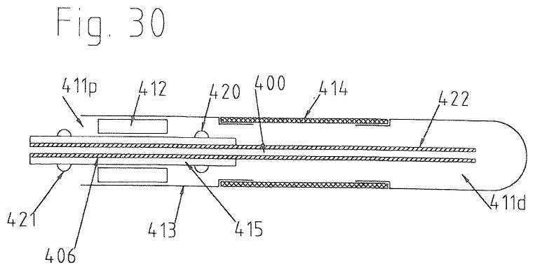

FIG. 30 presents an embodiment of the microelectrode comprising a hollow conductive element like the microelectrode of FIG. 4b but with a lateral member as the conductive bride. In addition to a hollow conductive element the microelectrode also comprises a third (421) and fourth (420) structural elements restricting axial movement of the conductive element, specifically the insulated portion of the conductive element. FIG. 30 illustrates a hollow conductive element (406) comprising a central conduit (400) providing liquid communication between the distal chamber (411d) and a reservoir (not shown) comprising a drug. The distal chamber (411d) comprises a lateral member of a net (414). The first structural component (412) is distinct from the casing (413). A proximal portion of conductive element is insulated (415). Third (421) and fourth (420) structural elements are disposed around the insulated portion of the conductive element. The third structural element (421) prohibits the tip of the non-insulated conductive element (422) from penetrating the casing of the distal chamber (411d). The fourth structural element (420) enables the removal of the casing when the microelectrode is removed from soft tissue by engagement of the conductive element located proximally and outside of the soft tissue.

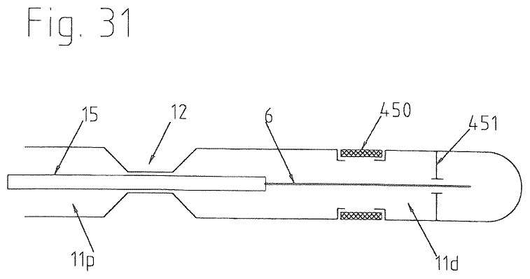

FIG. 31 illustrates an embodiment of the microelectrode comprising an electrically conductive bridge (450) in form of a lateral member presented as a net of conductive material. The lateral member electrically connects the distal chamber (11d) and the adjacent soft tissue. The first structural component forms an integral part of the casing (12). Also illustrated are the proximal compartment (11p), conductive element (6) and insulation of the conductive element (15).

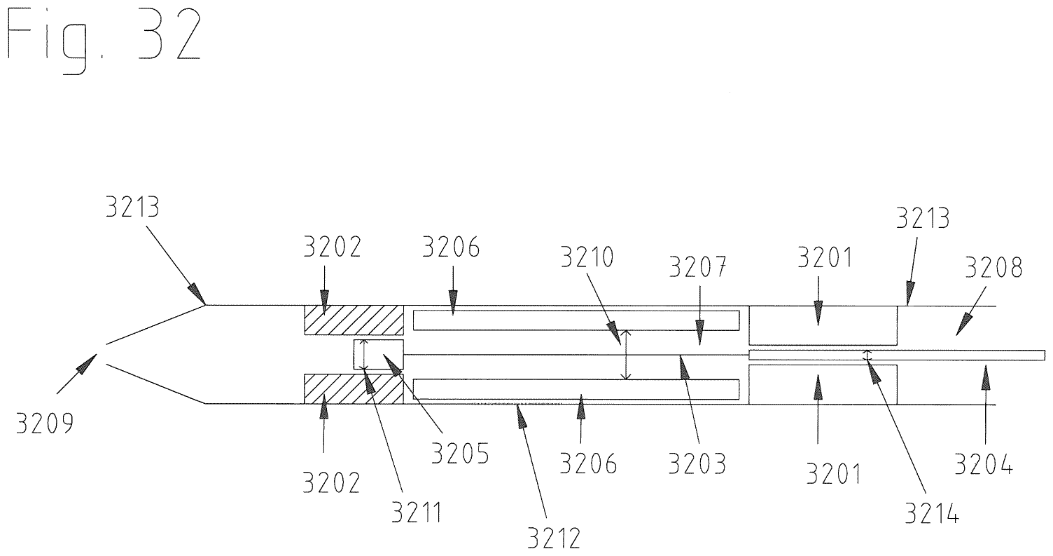

FIG. 32 illustrates a cross-sectional view of an embodiment of the microelectrode comprising a conductive element having a non-insulated portion between a distal and proximal insulated portion of a conductive element (3203). The diameter of the distal insulated portion of the conductive element (3205, 3211) is larger than the proximal insulated portion of the conductive element (3204, 2114). The microelectrode has a first (3201) and a second (3202) structural component slidably disposed around the proximal (3204) and distal (3205) insulated portions respectively. The non-insulated portion of the conductive element is disposed within an inner casing having an annular cross-section (3206). The inner casing has an inner diameter (3210) larger than the diameter (3211) of the distal insulated portion. The casing has an opening distally to the second structural component (3209). A conductive bridge (3212) in form of a lateral conductive sheet such as a mesh, net or ion-permeable membrane is comprised in the casing electrically coupling the non-insulated conductive element (2103) with soft tissue adjacent the conductive bridge when the microelectrode is implanted in soft tissue. The casing (3213), first structural component (3201) and second structural component (3202) form a distal chamber (3207). Part of the casing forms a proximal compartment (3208) around the proximal insulated portion of the conductive element (3204) proximally to the first structural component.

### Additional Embodiments

1. A microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber comprising electrically conductive bridges electrically coupling the distal chamber with the adjacent soft tissue, and wherein the casing comprises a first structural component in which the electrically insulated portion of the conductive element can slide in an axial direction.

2. The microelectrode according to claim 1, wherein the first structural component partitions the casing (envelope) in the distal chamber and a proximal compartment.

3. The microelectrode according to claim 1 or 2, wherein at least part of the electrically insulated portion is localized within the distal chamber.

4. The microelectrode according to any one of the preceding claims, wherein a lumen/void (enabling axial movements) is provided between the first structural component and the electrically insulated portion of the conductive element.

5. The microelectrode according to any one of the preceding claims, wherein the conductive filaments comprise metals, metal alloys and/or conductive polymers and/or carbon-containing materials such as graphene, graphite and carbon nanotubes.

6. The microelectrode according to any one of the preceding claims, wherein the conductive filaments are partly encased by an electrically insulating non-degradable material.

7. The microelectrode according to any one of the preceding claims, wherein the conductive filaments, or at least some of the conductive filaments comprise a conduit providing fluidic contact between the distal chamber and the adjacent soft tissue.

8. The microelectrode according to any of the preceding claims, wherein the first structural component has an extension in axial direction of at least from about 5 μm up to about 10 mm, preferably from about 5 μm up to about 3 mm.

9. The microelectrode according to any one of the preceding claims, wherein the innermost material(s) of the casing and/or the first structural components and/or the outermost material of the proximal electrically insulated portion of the conductive element is/are (each) selected to reduce friction.

10. The microelectrode according to any one of the preceding claims, wherein the distal chamber comprises a second structural component configured to reducing radial movement of the non-insulated portion of the conductive element relative to the distal casing, while also being configured to enable an axial movement of the non-insulated portion of the conductive element with respect to the second structural component.

11. The microelectrode according to claim 10, wherein the second structural component is an integral part of the casing.

12. The microelectrode according to any one of claim 10 or 11, wherein the second structural component is distinct from the casing being at least partly attached to the casing and configured to be slidably connected to or engaged with the non-insulated portion of the conductive element.

13. The microelectrode according to any one of the preceding claims, wherein the proximal electrically insulated portion of the conductive element comprises a section which facilitates flexing in radial and axial direction, suitably facilitates flexing in radial direction.

14. The microelectrode according to any one of the preceding claims, wherein the distal portion of the casing of the distal chamber has a three-dimensional shape narrowing in distal direction such as a spherical shape.

15, The microelectrode according to any one of the preceding claims, wherein a proximal portion of the distal chamber narrows down, preferably exhibiting an annular form forming the first structural component, in which the first structural component and the electrically insulated portion of the conductive element can slide in an axial direction.

16. The microelectrode according to any one of the preceding claims, wherein the friction between the casing and the adjacent soft tissue is higher than the friction between the innermost material of the casing and/or the first structural component and/or the outermost material of the proximal electrically insulated portion of the conductive element.

17. The microelectrode according to any one of the preceding claims, wherein the outermost material and/or outermost surface structure of the casing is selected to increase friction against the soft tissue.

18. The microelectrode according to any one of the preceding claims, wherein the casing comprises two layers of materials having an inner layer and an outer layer, wherein the material of inner layer is different from the material of the outer layer or wherein the surface structure of the inner layer is different from surface structure of the outer layer.

19. The microelectrode according to any one of the preceding claims, wherein the casing of the distal chamber comprises an engagement element, preferably comprised at the distal portion of the casing of the distal chamber, configured to reversibly engage with an elongated rigid pin such as a needle, the pin being configured to insert the microelectrode into the soft tissue or placing the microelectrode adjacent to soft tissue.

20. The microelectrode according to claim 19, wherein the engagement element is a loop or net.

21. The microelectrode according to any one of claim 19 or 20, wherein the engagement element is degradable in body fluids.

22. The microelectrode according to any one of claims 19 to 21, wherein the engagement element is a net established by micro- or nanofibers.

23. The microelectrode according to any one of the preceding claims, wherein the casing comprises means for increasing friction between the casing and the adjacent soft tissue.

24. The microelectrode according to claim 23, wherein the means for increasing friction is selected from micro- or nano-fibers attached to the outermost surface of the casing.

25. The microelectrode according to any one of the preceding claims, wherein a void/lumen between the first structural component and the outermost layer of the proximal electrically insulated portion of the conductive element comprises a composition facilitating the movement of the first structural component with respect to the outermost layer, particularly a composition comprising any one of lipids, hyaluronic acid, silicones (such as silicone oil or silicone grease) and a polymer of monosaccharides such as glucose and combinations thereof.

26. The microelectrode according to any one of the preceding claims, wherein the casing has a rotationally symmetric shape, suitably cylindrical shape.

27. The microelectrode according to any one of claims 2 to 26, wherein the diameter of the proximal compartment widens in a proximal direction.

28. The microelectrode according to any one of the preceding claims, wherein the distal chamber and optionally the proximal compartment comprises at least one biologically active substance such as a pharmaceutically active substance.

29. The microelectrode according to any one of the preceding claims, wherein the conductive element extending proximally of the proximal compartment is of a material or of materials different from that or those of the conductive element disposed in the proximal and distal compartments.

30. The microelectrode according to any one of the preceding claims, wherein the conductive element comprises conductive metals and/or conductive non-metal materials, such as conductive polymers.

31. The microelectrode according to any one of the preceding claims, wherein the electrically conductive element comprises or consists of materials selected from the group of platinum, iridium, gold, wolfram, stainless steel, amalgams of such materials, conductive polymers, carbon containing materials, such as graphene, graphite and carbon nanotubes.

32. The microelectrode according to any one of the preceding claims, wherein the electrically insulating material of the casing is a biocompatible, non-degradable flexible polymeric material, particularly a biocompatible, flexible polymer selected from polyurethanes, polyethylenes, polymers with a backbone comprising benzene (e.g. parylenes such as Parylene C and Parylene M), and polymers based on the polymerization of tetrafluoroethylene.

33. The microelectrode according to claim 32, wherein the electrically insulating material around the conductive element is selected from any one of the materials of claim 32 and electrically insulating flexible inorganic materials (such as glass or glass-like).

34. The microelectrode according to any one of the preceding claims, wherein the distal chamber, and optionally the proximal compartment, comprises a biocompatible material dissolvable or degradable in aqueous body fluids and providing structural support to the microelectrode when dry

35. The microelectrode according to any of the preceding claims, wherein the casing of the distal chamber has at least one opening providing (after implantation) a fluidic electrically conductive bridge between the non-insulated portion of the conductive element and the soft tissue enabling an exchange of ions between the distal chamber and the tissue.

36. The microelectrode according to any one of the preceding claims wherein the electrical impedance between the non-insulated portion of the conductive element and the soft tissue is lower than the electrical impedance inside the casing between the non-insulated portion of the conductive element and the tissue surrounding the proximal part of the proximal compartment or tissue proximally to the first structural component (in case there is no proximal compartment).

37. The microelectrode according to any one of the preceding claims, wherein the electrical impedance between the non-insulated portion of the conductive element and the soft tissue is at least 5 times lower, preferably at least 25 times lower, preferably at least 100 times lower, than the electrical impedance inside the casing between the non-insulated portion of the conductive element and the tissue surrounding the proximal part of the proximal compartment or tissue proximally to the first structural component.

38. The microelectrode according to any one of the preceding claims, wherein the first structural component and the proximal electrically insulated portion of the conductive element forms an annular channel, wherein the electrical impedance over the channel (when filled with body fluids) is at least 5 times higher, preferably at least 25 times higher, preferably at least 100 times higher than the electrical impedance between the non-insulated portion of the conductive element and the soft tissue and wherein the channel enables the first structural component to slide with respect to the conductive element in an axial direction.

39. The microelectrode according to any one of the preceding claims, wherein the perpendicular distance between the non-insulated portion of the conductive element and the at least one opening and or electrically conductive bridges in the casing of the distal chamber remains essentially the same during axial movements of the casing relative to the conductive element, optionally not more than 100%, preferably not more than 50%, no more than 20%, not more than 15%, preferably not more than 10%.

40. The microelectrode according to any one of claims 35 to 39, wherein the at least one opening has an area of at least about 1 μm².

41. The microelectrode according to any one of claims 35 to 40, wherein the distal chamber comprises a plurality of openings in the distal casing.

42. The microelectrode according to any one of claims 35 to 41, wherein the total area of all openings of the distal chamber is up to about 150000 μm² or more.

43. The microelectrode according to any one of the preceding claims, comprising a third structural element restricting axial movement of the casing with respect to the conductive element, suitably attached to the insulated portion of the conductive element, the third structural element positioned and configured to eliminate that the tip of the non-insulated portion of the conductive element penetrates the casing.

44. The microelectrode according to claim 43, wherein the third structural element constitutes an enlargement of the circumference of the insulated portion of the conductive element positioned on the insulated portion proximally with respect to the first structural component.

45. The microelectrode according to any one of the preceding claims, comprising a fourth structural element restricting axial movement of the casing with respect to the conductive element, suitably attached to the insulated portion of the conductive element, the fourth structural element positioned and configured to enable the engagement with the conductive element when the conductive element is removed from the soft tissue.

46. A microelectrode probe comprising a microelectrode as defined by any one of claims 1 to 45, wherein the distal chamber, and optionally the proximal compartment, comprise(s) a biocompatible material providing structural support to the probe when dry for insertion into soft tissue, wherein the biocompatible material is dissolvable or degradable in aqueous body fluids.

47. The microelectrode according to any one of claims 1 to 45, or microelectrode probe according to claim 46, wherein the microelectrode or microelectrode probe is embedded in an embedding matrix of a biocompatible material providing sufficient rigidity to the microelectrode or microelectrode probe when dry for insertion into soft tissue and dissolvable or degradable in aqueous body fluids.

48. The microelectrode according to any one of claims 1 to 45 or microelectrode probe according to claim 46 or 47, comprising an element holder, the proximal insulated electrically conductive element extending (in proximal direction) through the element holder, the holder configured to be secured to a tissue different from the soft tissue, in particular osseous or connective tissue.

49. The microelectrode according to any one of claims 1 to 45 and 47 and 48, or microelectrode probe according to any one of claims 46 to 48, wherein the electrically conductive element is electrically connected with an apparatus for registration of biological signals and stimulation of soft tissue.

50. The microelectrode probe according to any one of claims 46 and 47, wherein the biocompatible matrix-materials are selected from carbohydrate-based materials, protein-based materials, and non-natural polymeric materials or mixtures thereof.

51. An array of microelectrodes according to any one of claims 1 to 45 and 47 to 50, or an array of microelectrode probes according to any one of claims 46 to 50.

52. The array according to claim 51, wherein the microelectrodes and/or microelectrode probes are attached to a non-degradable matrix, preferably a distal section of the microelectrodes and/or microelectrode probes, preferably with the proviso that most of the electrically conductive bridges, preferably more than 50%, more than 60%, more than 70%, suitably essentially all, are not embedded by the non-degradable matrix.

53. The array according to claim 51 or 52, wherein the microelectrodes and/or microelectrode probes are adhesively attached to at least some of the micro. or nanofibers.

54. The array according to claim 53, wherein the micro- or nanofibers are non-degradable.

55. An array of microelectrodes and/or microelectrode probes configured to be at least partially embedded into or at least partially placed adjacent to soft tissue, in particular nervous, endocrine and muscle tissue, the microelectrodes and/or microelectrode probes comprising an electrically conductive element, the electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber having at least one opening providing (after implantation) a fluidic electrically conductive bridge between the non-insulated portion of the conductive element and the soft tissue enabling an exchange of ions between the distal chamber and the tissue, wherein the at least one opening is useful for recording and stimulation of electrically excitable cells, wherein the casing comprises a first structural component in which the electrically insulated portion of the conductive element can slide in an axial direction and wherein the microelectrodes are attached to a non-degradable matrix, preferably a distal section of the microelectrodes and/or microelectrode probes, preferably with the proviso that most of the openings, preferably more than 50%, more than 60%, more than 70%, suitably essentially all of the openings, are not embedded by the non-degradable matrix.

56 An array of microelectrodes and/or microelectrode probes configured to be at least partially embedded into or at least partially placed adjacent to soft tissue, in particular nervous, endocrine and muscle tissue, microelectrodes and/or microelectrode probes comprising an elongated electrically conductive element, the elongated electrically conductive element comprising a proximal electrically insulated portion and distal non-insulated portion, at least part of the conductive element being disposed in a casing (envelope) of electrically insulating non-degradable material, wherein the non-insulated portion of the element is encapsulated (surrounded) by the casing forming a distal chamber, in which the conductive element can slide in an axial direction, the casing of the distal chamber having at least one opening providing (after implantation) a fluidic electrically conductive bridge between the non-insulated portion of the conductive element and the soft tissue enabling an exchange of ions between the distal chamber and the tissue, wherein the at least one opening is useful for recording and stimulation of electrically excitable cells, wherein the casing comprises a first structural component in which the electrically insulated portion of the conductive element can slide in an axial direction, wherein the microelectrodes and/or microelectrode probes are adhesively attached to non-degradable micro or nanofibers.

57. The array according to claim 55 or 56, wherein the first structural component partitions the casing (envelope) in a distal chamber and a proximal compartment.

58. The array according to any one of claim 55 or 56, wherein at least part of the electrically insulated portion of the conductive element is localized within the distal chamber.

59. The array according to any one of claims 55 to 58, wherein a lumen/void (enabling axial movements) is provided between the first structural component and the electrically insulated portion of the conductive element.

60. The array according to any of claims 55 to 59, wherein the electrical impedance between the non-insulated portion of the conductive element and the soft tissue is lower than the electrical impedance inside the casing between the non-insulated portion of the conductive element and the tissue surrounding the proximal part of the proximal compartment or tissue proximally to the first structural component (in case there is no proximal compartment).

61. The array according to any one of claims 55 to 60, wherein the electrical impedance between the non-insulated portion of the conductive element and the soft tissue is at least 5 times lower, preferably at least 25 times lower, preferably at least 100 times lower, than the electrical impedance inside the casing between the non-insulated portion of the conductive element and the tissue surrounding the proximal part of the proximal compartment or tissue proximally to the first structural component.

62. The array according to any one of claims 55 to 61, wherein the first structural component and the proximal electrically insulated portion of the conductive element forms an annular channel, wherein the electrical impedance over the channel (when filled with body fluids) is at least 5 times higher, preferably at least 25 times higher, preferably at least 100 times higher than the electrical impedance between the non-insulated portion of the conductive element and the soft tissue and wherein the annular channel enables the first structural component to slide with respect to the conductive element in an axial direction.

63. The array according to any of claims 55 to 62, wherein the first structural component has an extension in axial direction of at least from about 5 μm up to about 10 mm, preferably from about 5 μm up to about 3 mm.

64. The array according to any one of claims 55 to 63, wherein the innermost material(s) of the casings and/or the first structural components and/or the outermost material of the proximal electrically insulated portion of the element is/are (each) selected to reduce friction.

65. The array according to claim any one of claims 55 to 64, wherein the perpendicular distance between the non-insulated portion of the conductive element and the at least one opening in the casing of the distal chamber remains essentially the same during axial movements of the casing relative to the conductive element, optionally not more than 100%, preferably not more than 50%, no more than 20%, not more than 15%, preferably not more than 10%.

66. The array according to any one of claims 55 to 65, wherein the distal chamber comprises a second structural component configured to reducing radial movement of the non-insulated portion of the conductive element relative to the distal casing, while also being configured to enable an axial movement of the non-isolated conductive element with respect to the second structural component.

67. The array according to claim 66, wherein the second structural component is an integral part of the casing.

68. The array according to claim 66 or 67, wherein the second structural component is distinct from the casing being at least partly attached to the casing and configured to be slidably connected to or engaged with the non-isolated conductive element.

69. The array according to any one of claims 55 to 68, wherein the at least one opening has an area of at least about 1 μm².

70. The array according to any one of claims 55 to 69, wherein the distal chambers comprises a plurality of openings in the distal casings.

71. The array according to any one of claims 55 to 70, wherein the maximum number of openings of the distal chambers is given by the maximum number of openings not significantly compromising the structural rigidity/conformation of the distal casings.

72. The array according to any one of claims 55 to 71, wherein the total area of all openings of the distal chamber is up to about 150000 μm² or more.

73. The array according to any one of claims 55 to 72, wherein the proximal electrically insulated portion of the conductive elements comprises a section which facilitates flexing in radial and axial direction, suitably facilitates flexing in radial direction.

74. The array according to claim 51, 55 or 56 wherein the microelectrodes are disposed essentially in parallel and essentially in (as) one sheet.

75. The array according to claim 74, wherein the microelectrodes are arranged such that most of the at least one opening and/or the electrically conductive bridges of the distal chamber faces in essentially the same direction.

76. The array according to any one of claims 51 to 75, the microelectrodes and/or microelectrode probes partially or entirely embedded in an array matrix of a biocompatible material providing sufficient rigidity to the array when dry for insertion into soft tissue and dissolvable or degradable in aqueous body fluids.

77. The array according to claim 76, wherein the biocompatible dissolvable or degradable materials are selected from carbohydrate-based materials, protein-based materials, and non-natural polymeric materials, and mixtures thereof.

78. The array according to any one of claims 51 to 77, comprising an array cover.

79. The array according to claim 78, wherein the array matrix extends to the distal face of the array cover.

80. The array according to any one of claims 51 to 79, further comprising an array casing of a flexible, non-degradable material embracing a part of the array matrix.

81. The array according to claim 80, embedded in an outer array matrix of a biocompatible material which is solid when dry and dissolvable or degradable in aqueous body fluids.

82. The array according to claim 81, wherein the outer array matrix comprises a biocompatible material selected from carbohydrate-based materials, protein-based materials, and non-natural polymeric materials, and mixtures.

83. The array according to any one of claims 55 to 73, wherein the casing of microelectrodes comprises means for increasing friction between the casing and the adjacent soft tissue.

84. A method for manufacturing the microelectrode according to any one of claims 1 to 45 and 47 to 50, or microelectrode probe of any one of claims 46 to 50, comprising:
providing an elongated electrically conductive element,
covering a proximal portion of the element with an electrically insulating layer thereby providing a proximal electrically insulated portion and a distal non-insulated portion of the conductive element;
forming a distal matrix dissolvable or degradable in aqueous body fluids extending axially around the distal non-insulated portion of the conductive element, and optionally extending in a distal direction from the distal non-insulated portion of the conductive element;
applying a sliding facilitating composition to a section of the insulated element proximally with respect to the distal matrix and distally with respect to an optional proximal matrix wherein the sliding facilitating composition is facilitating the axial movement of a first layer of electrically insulating non-degradable material with respect to the insulating layer of the conductive element, said medium optionally providing for a sufficient void/lumen between the insulating layer of the conductive element and first layer of electrically insulating non-degradable material;
applying electrically conductive bridges to at least a part of the surface of the distal matrix (20d) which is not cut away,
optionally forming a proximal matrix extending axially around at least part of the proximal electrically insulated portion of the conductive element;
covering the distal matrix and at least part of the proximal electrically insulated portion of the conductive element with a first layer of electrically insulating non-degradable material, thereby providing a casing encapsulating the distal non-insulated portion of the element forming a distal chamber and a first structural component
cutting part of the non-insulated portion of the conductive element and first layer of electrically insulating non-degradable material near the distal end of the distal matrix (distal end of the distal chamber) comprising the distal non-insulated portion of the electrically conductive element, thereby providing a distal opening of the distal compartment.
applying a further distal tip matrix distally to the distal opening,
covering the tip matrix and at least part of the first layer of electrically insulating non-degradable material with a second layer of electrically insulating non-degradable material, thereby forming a distal end cap part forming part of the casing of the distal chamber
wherein the distal and optionally proximal matrices provide structural support to the microelectrode or probe when dry for insertion into soft tissue and;
and wherein: at least an area of each electrically conductive bridge is not covered by first layer and optional second layer of electrically insulating non-degradable material,
and/or one opening through the first layer and optional second layer of the casing of the distal chamber is provided,
and/or the conductive bridge protruding radially from the casing covered with first layer and optional second layer is modified such that the conductive bridge provides an electrical coupling between the distal chamber and any soft tissue adjacent the microelectrode (once the microelectrode is positioned in soft tissue).

85. A method for manufacturing the microelectrode according to any one of claims 2 to 45 and 47 to 50, or microelectrode probe of any one of claims 46 to 50, comprising:
providing an elongated electrically conductive element,
covering a proximal portion of the element with an electrically insulating layer thereby providing a proximal electrically insulated portion and a distal non-insulated portion of the conductive element;
forming a distal matrix dissolvable or degradable in aqueous body fluid extending axially around, and optionally extending in a distal direction from the distal non-insulated portion of the conductive element;
applying electrically conductive bridges to at least a part of the surface of the distal matrix (20d) which is not cut away,
forming a proximal matrix extending axially around at least part of the proximal electrically insulated portion of the conductive element and thereby forming an intermediate section of the insulated conductive element with an axial extension, the intermediate section positioned proximally to the distal matrix and distally to the proximal matrix not covered by the distal and proximal matrices;
applying a thin (up to about 5 μm) layer of a first intermediate matrix and/or sliding facilitating composition to the intermediate section of the insulated element facilitating the axial movement of a first layer of electrically insulating non-degradable material with respect to the insulating layer of the conducting element, said first intermediate matrix and/or composition providing for a sufficient void/lumen (annular channel) between the electrically insulated portion of the conductive element and the first layer of electrically insulating non-degradable material;
covering distal, proximal matrices and the intermediate section of the proximal electrically insulated portion of the element, the intermediate section comprising an intermediate matrix and/or sliding facilitating composition, with a first layer of electrically insulating non-degradable material, thereby providing a casing comprising a distal chamber, a first structural component and a proximal compartment;
optionally providing a second intermediate matrix on the first layer of electrically insulating non-degradable material in the constriction in radial direction of the first layer between the distal chamber and proximal compartment;
cutting part of the distal non-insulated portion of the electrically conductive element and the first layer of electrically insulating material near the distal end of the distal matrix (distal end of the distal chamber), thereby providing a distal opening of the distal chamber;
applying a further distal tip matrix distally to the distal opening;
covering the distal tip matrix and at least part of the first layer with a second layer of electrically insulating material thereby forming a distal end cap forming part of the casing of the distal chamber;
and removing the first layer and optionally second layer at a circumferential annular zone of the proximal matrix;
wherein the distal matrix, distal tip matrix, proximal matrix and optionally first and second intermediate matrices are of a biocompatible material providing sufficient rigidity to the probe when dry for insertion into soft tissue and dissolvable or degradable in aqueous body fluids;
and wherein: at least an area of each electrically conductive bridge is not covered by first layer and optional second layer of electrically insulating non-degradable material,
and/or one opening is provided through the first layer and optional second layer of the casing of the distal chamber, and/or the conductive bridge protruding radially from the casing covered with first layer and optional second layer is modified such that the conductive bridge provides an electrical coupling between the distal chamber and any soft tissue adjacent the microelectrode (once the microelectrode is positioned in soft tissue).

86. A method for manufacturing the microelectrode according to any one of claims 1 to 45 and 47 to 50, or microelectrode probe according to any one of claims 46 to 50, comprising:
providing an elongated electrically conductive element,
covering a proximal portion of the element with an electrically insulating layer thereby providing a proximal electrically insulated portion and a distal non-insulated portion of the element;
providing a first structural component configured to enable an axial movement with respect to the proximal electrically insulated portion of the conductive element;
positioning the first structural component around the proximal electrically insulated portion of the conductive element, suitably at a certain axial distance from the distal non-insulated portion of the conductive element;
applying a proximal matrix dissolvable or degradable in aqueous body fluids around the proximal electrically insulated portion of the conductive element, the proximal matrix extending from the proximal face of the first structural component in proximal direction;
applying a distal matrix dissolvable or degradable in aqueous body fluids around the distal non-insulated portion of the element extending from the distal face of the first structural component in distal direction, and extending in a distal direction from the distal non-insulated portion of the conductive element, suitably up to several millimeters;
applying electrically conductive bridges to at least a part of the surface of the distal matrix (20d) which is not cut away,
applying a first layer of electrically insulating non-degradable material on the proximal and distal matrices and the circumference of the first structural component, thereby forming a casing comprising a distal chamber and a proximal compartment; and wherein: at least an area of each electrically conductive bridge is not covered by first layer and optional second layer of electrically insulating non-degradable, and/or one opening is provided through the first layer and optional second layer of the casing of the distal,
and/or the conductive bridge protruding radially from the casing covered with first layer and optionally second layer is modified such that the conductive bridge provides an electrical coupling between the distal chamber and any soft tissue adjacent the microelectrode (once the microelectrode is positioned in soft tissue).

87. The method of claim 86, wherein the proximal matrix widens in a proximal direction.

88. The method of any one of claims 84 to 87 wherein the electrically conductive bridges applied to at least a part of the surface of the distal matrix are disposed in an essentially radial direction with respect to the main axis of the conductive element.

89. A microelectrode comprising a conductive element comprising distal and proximal non-insulated sections and further an insulated section between said distal and proximal non-insulated section, where the insulated section of the conductive element is slidably associated with a casing encapsulating at least the proximal non-insulated section of the conductive element, the casing further insulating the proximal non-insulated section of the conductive element from direct contact with adjacent soft tissue once the microelectrode is inserted into soft tissue forming a proximal lumen, the casing exposing conductive material capable of electrically coupling the proximal non-insulated section of the conductive element with a second conductive element electrically coupled to the conductive material of the casing.

1. A microelectrode configured to be at least partially embedded into or at least partially placed adjacent to soft tissue, in particular nervous, endocrine and muscle tissue, comprising an electrically conductive element, the electrically conductive element comprising a proximal electrically insulated portion and distal non-insulated portion, at least part of the conductive element being disposed in a casing of electrically insulating non-degradable material comprising a first structural component and optionally a second structural component, the distal non-insulated portion of the conductive element being encapsulated (surrounded) by the casing forming a distal chamber, a void/lumen being present between the insulated portion of the conductive element and the first structural component the void/lumen enabling the conductive element to slide with respect to the casing, wherein the casing of the distal chamber comprises at least one electrically conductive bridge electrically coupling the distal chamber with the adjacent soft tissue.

2. The microelectrode according to claim 1, wherein the electrically conductive bridge is selected from a lateral member such as a conducive mesh, net, web, ion permeable membranes, porous polymeric materials a latera member, filament-like structures penetrating the casing of the distal chamber, and a conductive bridge comprises surface area in electrical contact with fluid inside the distal chamber and the adjacent soft tissue, said surface area having sufficient extension for providing an efficient electrical coupling.

3. The microelectrode according to claim 1, wherein the electrically conductive bridge is selected from filament-like structures penetrating the casing of the distal chamber.

4. The microelectrode according to claim 1, wherein the electrically conductive bridge is hollow.

5. The microelectrode according to claim 1, wherein the electrically conductive bridge is positioned laterally with respect to the casing of the distal chamber.

6. A microelectrode configured to be at least partially embedded into or at least partially placed adjacent to soft tissue, in particular nervous, endocrine and muscle tissue, comprising an elongated electrically conductive element comprising a proximal and distal end, the electrically conductive element comprising insulated proximal and distal portions, the proximal insulated portion extending in distal direction from the proximal end, the distal insulated portion extending in proximal direction from the distal end, the proximal and distal portions separated by a non-insulated portion of the conductive element, at least the non-insulated portion of the conductive element being essentially centrally disposed in a casing of an electrically insulating non-degradable material, the microelectrode further comprising first and second structural components, said first and second structural components extending in radial direction between the casing and the conductive element, the first structural component movably disposed around the proximal insulated portion of the conductive element, the second structural component movably disposed around the distal insulated portion of the conductive element; the casing, first and second structural components forming a distal chamber, wherein the casing has an opening distally to the second structural component, wherein the casing is configured to electrically couple the non-insulated portion of the conductive element with the soft tissue.

7. The microelectrode according to claim 6, wherein at least part of the non-insulated portion of the conductive element is disposed in an inner casing of an electrically conductive material, the inner casing having an outer diameter which is equal to or smaller than the inner diameter of the casing.

8. The microelectrode according to claim 7, wherein the inner casing has an annular cross-section having an inner diameter larger than any of the diameters of the insulated proximal and distal portions of the conductive element.

9. The microelectrode according to claim 1, wherein the first and second structural components are permanently attached to the casing.

10. The microelectrode according to claim 1, wherein first and second structural components are permanently attached to the casing prohibiting electrical currents between said first and second structural components and the casing.

11. The microelectrode according to claim 1, wherein the diameter of the distal insulated portion of the conductive element is larger than the inner diameter of the first structural component.

12. The microelectrode according to claim 11, wherein the inner casing has an annular cross-section having an inner diameter larger than the distal insulated portion of the conductive element.

13. The microelectrode according to claim 6, wherein the first and second structural components have annular cross-sections where the inner diameter of the first structural component is larger than the diameter of the proximal insulated portion of the conductive element, and that the inner diameter of the second structural component is larger than the diameter of the distal insulated portion of the conductive element.

14. The microelectrode according to claim 1, wherein the electrical coupling of the non-insulated portion of the conductive element and the soft tissue is provided by at least one conductive bridge.

15. The microelectrode according to claim 14, wherein the conductive bridge is selected from a fluidic conductive bridge and/or an electrically conductive bridge.

16. The microelectrode according to claim 14, wherein the conductive bridge is selected from an opening, a conductive filament-like structure penetrating the casing of the distal chamber, a lateral member such as a conducive mesh, net, web, and ion permeable membranes, porous polymeric materials.

17. The microelectrode according to claim 1, wherein first structural component partitions the casing in a distal chamber and a proximal compartment.

18. The microelectrode according to claim 1, wherein the electrically conductive element comprises or consists of materials selected from the group of platinum, iridium, gold, wolfram, stainless steel, amalgams of such materials, conductive polymers, carbon containing materials, such as graphene, graphite and carbon nanotubes.

19. The microelectrode according to claim 1, wherein the first and optional second structural components are separate from the casing, preferably made of electrically insulating, non-degradable materials.

20. The microelectrode according to claim 1, wherein the distal section of the casing has a three-dimensional shape narrowing down in distal direction, preferably narrowing down distally to the second structural component.

21. The microelectrode according to claim 1, wherein the friction between the casing and the adjacent soft tissue is higher than the friction either a) between the casing and first and optional second structural component or b) between the proximally insulated portion of the conductive element and the first structural component and optionally between the second structural component and the second structural component.

22. The microelectrode according to claim 1, wherein the casing comprises means for increasing friction between the casing and the adjacent soft tissue preferably selected from micro- or nano-fibers attached to the outermost surface of the casing.

23. The microelectrode according to claim 1, wherein the casing has a rotationally symmetric shape, suitably cylindrical shape.

24. The microelectrode according to claim 1, wherein the distal chamber and optionally the proximal compartment comprises at least one biologically active substance such as a pharmaceutically active substance.

25. The microelectrode according to claim 1, wherein the electrically insulating material of the casing is a biocompatible, non-degradable flexible polymeric material, particularly a biocompatible, flexible polymer preferably selected from polyurethanes, polyethylenes, polymers with a backbone comprising benzene (e.g. parylenes such as Parylene C and Parylene M), and polymers based on the polymerization of tetrafluoroethylene.

26. The microelectrode according to claim 1, wherein the distal chamber, and optionally the proximal compartment, comprises a biocompatible material dissolvable or degradable in aqueous body fluids and providing structural support to the microelectrode when dry.

27. The microelectrode according to claim 1, wherein the microelectrode when implanted, the electrical impedance between the non-insulated portion of the conductive element and the soft tissue adjacent conductive bridges is lower, preferably at least 5 times lower, 25 times lower, 100 times lower, than the electrical impedance inside the casing between the non-insulated portion of the conductive element and the tissue surrounding the proximal part of the proximal compartment or tissue proximally to the first structural component (in case there is no proximal compartment) and lower, preferably at least 5 times lower, 25 times lower, 100 times lower, than the electrical impedance inside the casing between the non-insulated portion of the conductive element and the tissue surrounding the opening in the casing distally to the second conductive component.

28. A microelectrode probe comprising a microelectrode as defined by claim 1, wherein the distal chamber, and optionally the proximal compartment, comprise(s) a biocompatible material providing structural support to the probe when dry for insertion into soft tissue, wherein the biocompatible material is dissolvable or degradable in aqueous body fluids.

29. An array of microelectrodes according to claim 1.

30. A method for manufacturing the microelectrode according to claim 1, comprising:
providing an elongated electrically conductive element,
covering a proximal portion of the element with an electrically insulating layer thereby providing a distal and proximal electrically insulated portion and a non-insulated portion of the conductive element between the distal and proximal insulated portions;
forming a distal matrix dissolvable or degradable in aqueous body fluids extending axially around the distal non-insulated portion of the conductive element, and optionally extending in a distal direction from the distal non-insulated portion of the conductive element;
applying a sliding facilitating composition to a section of the insulated element proximally with respect to the distal matrix and distally with respect to an optional proximal matrix wherein the sliding facilitating composition is facilitating the axial movement of a first layer of electrically insulating non-degradable material with respect to the insulating layer of the conductive element, said medium optionally providing for a sufficient void/lumen between the insulating layer of the conductive element and first layer of electrically insulating non-degradable material;
optionally applying filament-like structures to at least a part of the surface of the distal matrix (20d) protruding from the distal matrix in an essentially radial direction which is not cut away,
optionally forming a proximal matrix extending axially around at least part of the proximal electrically insulated portion of the conductive element;
covering the distal matrix and at least part of the proximal electrically insulated portion of the conductive element with a first layer of electrically insulating non-degradable material, thereby providing a casing encapsulating the distal non-insulated portion of the element forming a distal chamber and a first structural component
cutting part of the non-insulated portion of the conductive element and first layer of electrically insulating non-degradable material near the distal end of the distal matrix (distal end of the distal chamber) comprising the distal non-insulated portion of the electrically conductive element, thereby providing a distal opening of the distal compartment
removing a lateral portion of the first layer of electrically insulating non-degradable material making up the casing of the distal chamber;
covering the lateral portion with a lateral member selected from any one of a porous polymeric material, mesh, net and/or web,
applying a further distal tip matrix distally to the distal opening,
covering the tip matrix and at least part of the first layer of electrically insulating non-degradable material with a second layer of electrically insulating non-degradable material, thereby forming a distal end cap part forming part of the casing of the distal chamber
where the distal and optionally proximal matrices provide structural support to the microelectrode or probe when dry for insertion into soft tissue and where optionally at least an area of each filament-like structures is not covered by first layer and optional second layer of electrically insulating non-degradable material,
and where optionally one opening through the first layer and optional second layer of the casing of the distal chamber is provided,
and optional the filament-like structures protruding radially from the casing covered with first layer and optional second layer are modified such that the conductive bridge provides an electrical coupling between the distal chamber and any soft tissue adjacent the microelectrode (once the microelectrode is positioned in soft tissue).

31. A method for manufacturing the microelectrode according to claim 1, or microelectrode probe according to claim 28, comprising:
providing a conductive element and insulating the element with a polymer such as Parylene C and de-insulating, by e.g. laser milling, a defined section of the conductive element, thereby forming a distal and proximal insulated portion and a non-insulated portion between distal and proximal insulated portions of the conductive element;
providing a first and second structural components of non-conductive materials with annular shape, the first structural component having an inner diameter larger than the outer diameter of the proximal insulated portion of the conductive element, the second structural component having an inner diameter larger than the outer diameter of the distal insulated portion of the conductive element;
providing an inner casing of annular shape of a conductive material having an inner diameter larger than any one of the diameters of the insulated proximal and distal portions of the conductive element, the inner casing comprising radial conductive bridges such as radial protrusions;
aligning the first structural component, inner casing and second structural components and inserting the conductive element through first structural components, second structural component and inner casing, such that at least part of the inner casing is positioned between first and second structural component, and that at least part of the first structural component is disposed around the proximal insulated portion of the conductive element, and that at least part of the second structural component is disposed around the distal insulated portion of the conductive element, and that the non-insulated portion of the conductive element is disposed within the inner casing;
applying to the proximal portion of the conductive element proximally to the first structural component and in proximal direction a dissolvable matrix material and applying a cone shaped of a dissolvable matrix material distally to the second structural component thereby forming a pre-microelectrode:
providing a coating of an electrically insulating non-degradable material around the pre-microelectrode thereby forming a casing and a distal chamber formed by the casing, first and second structural components;
cutting the coat distally to the second structural component, thereby providing an opening in the casing;
cutting the radial conductive bridges thereby providing conductive bridges electrically coupling the non-insulated portion of the conductive element with adjacent soft tissue when the microelectrode is disposed into soft tissue.
